SEQUENCE LISTING (569 sequences)

SEQ ID NO: 1 - protein, 879 aa, artificial sequence
  PTGFRN
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2 - protein, 731 aa, artificial sequence
  PTGFRN Protein Fragment #1
PSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 3 - protein, 611 aa, artificial sequence
  PTGFRN Protein Fragment #3
VATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 4 - protein, 485 aa, artificial sequence
  PTGFRN Protein Fragment #3
SPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 5 - protein, 343 aa, artificial sequence
  PTGFRN Protein Fragment #4
KPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 6 - protein, 217 aa, artificial sequence
  PTGFRN Protein Fragment #5
VRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 7 - protein, 66 aa, artificial sequence
  PTGFRN Protein Fragment #6
SKPVFITVKMDVLNAFKYPLLIGVGLSTVIGLLSCLIGYCSSHWCCKKEVQETRRERRRLMSMEMD

SEQ ID NO: 8 - protein, 21 aa, artificial sequence
  PTGFRN Protein Fragment #7
MGRLASRPLLLALLSLALCRG

SEQ ID NO: 9-10 - skipped (2 entries)

SEQ ID NO: 11 - protein, 219 aa, artificial sequence
  Human IL-12A Subunit
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 12 - protein, 328 aa, artificial sequence
  Human IL-12B Subunit
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 13 - protein, 548 aa, artificial sequence
  IL-12 Fusion (signal peptide-p40-linker-p35)
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSGGGSGGGGSGGGGSGGGSGGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 14-32 - skipped (19 entries)

SEQ ID NO: 33 - protein, 192 aa, artificial sequence
  The PTGFRN protein Fragment
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEM

SEQ ID NO: 34-400 - skipped (367 entries)

SEQ ID NO: 401 - protein, 332 aa, artificial sequence
  The MARCKS protein
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 402 - protein, 195 aa, artificial sequence
  The MARCKSL1 protein
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 403 - protein, 227 aa, artificial sequence
  The BASP1 protein
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 404 - protein, 8 aa, artificial sequence
  Synthetic Peptide
  misc_Feature (2)..(2): Wherein Xaa is Alanine or any other amino acid
GXKLSKKK

SEQ ID NO: 405 - protein, 4 aa, artificial sequence
  effector domain
KKKK

SEQ ID NO: 406 - protein, 5 aa, artificial sequence
  effector domain
KKKKK

SEQ ID NO: 407 - protein, 4 aa, artificial sequence
  effector domain
RRRR

SEQ ID NO: 408 - protein, 5 aa, artificial sequence
  effector domain
RRRRR

SEQ ID NO: 409 - protein, 4 aa, artificial sequence
  effector domain
  misc_feature (1)..(4): Wherein Xaa can be either Lys or Arg
XXXX

SEQ ID NO: 410 - protein, 5 aa, artificial sequence
  effector domain
  misc_feature (1)..(5): Wherein Xaa can be either Lys or Arg
XXXXX

SEQ ID NO: 411 - protein, 7 aa, artificial sequence
  Scaffold Y protein
GGKLSKK

SEQ ID NO: 412 - protein, 7 aa, artificial sequence
  Scaffold Y protein
GAKLSKK

SEQ ID NO: 413 - protein, 7 aa, artificial sequence
  Scaffold Y protein
GGKQSKK

SEQ ID NO: 414 - protein, 7 aa, artificial sequence
  Scaffold Y protein
GGKLAKK

SEQ ID NO: 415 - protein, 6 aa, artificial sequence
  N-terminus domain
GGKLSK

SEQ ID NO: 416 - protein, 6 aa, artificial sequence
  N-terminus domain
GAKLSK

SEQ ID NO: 417 - protein, 6 aa, artificial sequence
  N-terminus domain
GGKQSK

SEQ ID NO: 418 - protein, 6 aa, artificial sequence
  N-terminus domain
GGKLAK

SEQ ID NO: 419 - protein, 4 aa, artificial sequence
  effector domain
KKKG

SEQ ID NO: 420 - protein, 5 aa, artificial sequence
  effector domain
KKKGY

SEQ ID NO: 421 - protein, 6 aa, artificial sequence
  effector domain
KKKGYN

SEQ ID NO: 422 - protein, 7 aa, artificial sequence
  effector domain
KKKGYNV

SEQ ID NO: 423 - protein, 8 aa, artificial sequence
  effector domain
KKKGYNVN

SEQ ID NO: 424 - protein, 6 aa, artificial sequence
  effector domain
KKKGYS

SEQ ID NO: 425 - protein, 6 aa, artificial sequence
  effector domain
KKKGYG

SEQ ID NO: 426 - protein, 6 aa, artificial sequence
  effector domain
KKKGYG

SEQ ID NO: 427 - protein, 5 aa, artificial sequence
  effector domain
KKKGS

SEQ ID NO: 428 - protein, 5 aa, artificial sequence
  effector domain
KKKGS

SEQ ID NO: 429 - protein, 7 aa, artificial sequence
  effector domain
KKKGSGS

SEQ ID NO: 430 - protein, 4 aa, artificial sequence
  effector domain
KKKS

SEQ ID NO: 431 - protein, 5 aa, artificial sequence
  effector domain
KKKSG

SEQ ID NO: 432 - protein, 6 aa, artificial sequence
  effector domain
KKKSGG

SEQ ID NO: 433 - protein, 7 aa, artificial sequence
  effector domain
KKKSGGS

SEQ ID NO: 434 - protein, 8 aa, artificial sequence
  effector domain
KKKSGGSG

SEQ ID NO: 435 - protein, 8 aa, artificial sequence
  effector domain
KKSGGSGG

SEQ ID NO: 436 - protein, 10 aa, artificial sequence
  effector domain
KKKSGGSGGS

SEQ ID NO: 437 - protein, 8 aa, artificial sequence
  effector domain
KRFSFKKS

SEQ ID NO: 438 - protein, 8 aa, artificial sequence
  Scaffold Protein
GGKLSKKK

SEQ ID NO: 439 - protein, 8 aa, artificial sequence
  Scaffold Y protein
GGKLSKKS

SEQ ID NO: 440 - protein, 8 aa, artificial sequence
  Scaffold Y protein
GAKLSKKK

SEQ ID NO: 441 - protein, 8 aa, artificial sequence
  Scaffold Y protein
GAKLSKKS

SEQ ID NO: 442 - protein, 8 aa, artificial sequence
  Scaffold Y protein
GGKQSKKK

SEQ ID NO: 443 - protein, 8 aa, artificial sequence
  Scaffold Y protein
GGKQSKKK

SEQ ID NO: 444 - protein, 8 aa, artificial sequence
  Scaffold Y protein
GGKLAKKK

SEQ ID NO: 445 - protein, 8 aa, artificial sequence
  Scaffold Y protein
GGKLAKKS

SEQ ID NO: 446 - protein, 14 aa, artificial sequence
  Scaffold Y protein
GGKLSKKKKGYNVN

SEQ ID NO: 447 - protein, 14 aa, artificial sequence
  Scaffold Y protein
GAKLSKKKKGYNVN

SEQ ID NO: 448 - protein, 14 aa, artificial sequence
  Scaffold Y protein
GGKQSKKKKGYNVN

SEQ ID NO: 449 - protein, 14 aa, artificial sequence
  Scaffold Y protein
GGKLAKKKKGYNVN

SEQ ID NO: 450 - protein, 14 aa, artificial sequence
  Scaffold Y protein
GGKLSKKKKGYSGG

SEQ ID NO: 451 - protein, 14 aa, artificial sequence
  Scaffold Y protein
GGKLSKKKKGSGGS

SEQ ID NO: 452 - protein, 14 aa, artificial sequence
  Scaffold Y protein
GGKLSKKKKSGGSG

SEQ ID NO: 453 - protein, 14 aa, artificial sequence
  Scaffold Y protein
GGKLSKKKSGGSGG

SEQ ID NO: 454 - protein, 14 aa, artificial sequence
  Scaffold Y protein
GGKLSKKSGGSGGS

SEQ ID NO: 455 - protein, 14 aa, artificial sequence
  Scaffold Y protein
GGKLSKSGGSGGSV

SEQ ID NO: 456 - protein, 14 aa, artificial sequence
  Scaffold Y protein
GAKKSKKRFSFKKS

SEQ ID NO: 457 - protein, 29 aa, artificial sequence
  Scaffold Y protein
GGKLSKKKKGYNVNDEKAKEKDKKAEGAA

SEQ ID NO: 458 - protein, 28 aa, artificial sequence
  Scaffold Y protein
GGKLSKKKKGYNVNDEKAKEKDKKAEGA

SEQ ID NO: 459 - protein, 27 aa, artificial sequence
  Scaffold Y protein
GGKLSKKKKGYNVNDEKAKEKDKKAEG

SEQ ID NO: 460 - protein, 26 aa, artificial sequence
  Scaffold Y protein
GGKLSKKKKGYNVNDEKAKEKDKKAE

SEQ ID NO: 461 - protein, 25 aa, artificial sequence
  Scaffold Y protein
GGKLSKKKKGYNVNDEKAKEKDKKA

SEQ ID NO: 462 - protein, 24 aa, artificial sequence
  Scaffold Y protein
GGKLSKKKKGYNVNDEKAKEKDKK

SEQ ID NO: 463 - protein, 23 aa, artificial sequence
  Scaffold Y protein
GGKLSKKKKGYNVNDEKAKEKDK

SEQ ID NO: 464 - protein, 22 aa, artificial sequence
  Scaffold Y protein
GGKLSKKKKGYNVNDEKAKEKD

SEQ ID NO: 465 - protein, 21 aa, artificial sequence
  Scaffold Y protein
GGKLSKKKKGYNVNDEKAKEK

SEQ ID NO: 466 - protein, 20 aa, artificial sequence
  Scaffold Y protein
GGKLSKKKKGYNVNDEKAKE

SEQ ID NO: 467 - protein, 19 aa, artificial sequence
  Scaffold Y protein
GGKLSKKKKGYNVNDEKAK

SEQ ID NO: 468 - protein, 18 aa, artificial sequence
  Scaffold Y protein
GGKLSKKKKGYNVNDEKA

SEQ ID NO: 469 - protein, 17 aa, artificial sequence
  Scaffold Y protein
GGKLSKKKKGYNVNDEK

SEQ ID NO: 470 - protein, 16 aa, artificial sequence
  Scaffold Y protein
GGKLSKKKKGYNVNDE

SEQ ID NO: 471 - protein, 15 aa, artificial sequence
  Scaffold Y protein
GGKLSKKKKGYNVND

SEQ ID NO: 472 - protein, 13 aa, artificial sequence
  Scaffold Y protein
GGKLSKKKKGYNV

SEQ ID NO: 473 - protein, 12 aa, artificial sequence
  Scaffold Y protein
GGKLSKKKKGYN

SEQ ID NO: 474 - protein, 11 aa, artificial sequence
  Scaffold Y protein
GGKLSKKKKGY

SEQ ID NO: 475 - protein, 10 aa, artificial sequence
  Scaffold Y protein
GGKLSKKKKG

SEQ ID NO: 476 - protein, 9 aa, artificial sequence
  Scaffold Y protein
GGKLSKKKK

SEQ ID NO: 477 - protein, 29 aa, artificial sequence
  Scaffold Y protein
GAKKSKKRFSFKKSFKLSGFSFKKNKKEA

SEQ ID NO: 478 - protein, 28 aa, artificial sequence
  Scaffold Y protein
GAKKSKKRFSFKKSFKLSGFSFKKNKKE

SEQ ID NO: 479 - protein, 27 aa, artificial sequence
  Scaffold Y protein
GAKKSKKRFSFKKSFKLSGFSFKKNKK

SEQ ID NO: 480 - protein, 26 aa, artificial sequence
  Scaffold Y protein
GAKKSKKRFSFKKSFKLSGFSFKKNK

SEQ ID NO: 481 - protein, 25 aa, artificial sequence
  Scaffold Y protein
GAKKSKKRFSFKKSFKLSGFSFKKN

SEQ ID NO: 482 - protein, 24 aa, artificial sequence
  Scaffold Y protein
GAKKSKKRFSFKKSFKLSGFSFKK

SEQ ID NO: 483 - protein, 23 aa, artificial sequence
  Scaffold Y protein
GAKKSKKRFSFKKSFKLSGFSFK

SEQ ID NO: 484 - protein, 22 aa, artificial sequence
  Scaffold Y protein
GAKKSKKRFSFKKSFKLSGFSF

SEQ ID NO: 485 - protein, 21 aa, artificial sequence
  Scaffold Y protein
GAKKSKKRFSFKKSFKLSGFS

SEQ ID NO: 486 - protein, 20 aa, artificial sequence
  Scaffold Y protein
GAKKSKKRFSFKKSFKLSGF

SEQ ID NO: 487 - protein, 19 aa, artificial sequence
  Scaffold Y protein
GAKKSKKRFSFKKSFKLSG

SEQ ID NO: 488 - protein, 18 aa, artificial sequence
  Scaffold Y protein
GAKKSKKRFSFKKSFKLS

SEQ ID NO: 489 - protein, 17 aa, artificial sequence
  Scaffold Y protein
GAKKSKKRFSFKKSFKL

SEQ ID NO: 490 - protein, 16 aa, artificial sequence
  Scaffold Y protein
GAKKSKKRFSFKKSFK

SEQ ID NO: 491 - protein, 15 aa, artificial sequence
  Scaffold Y protein
GAKKSKKRFSFKKSF

SEQ ID NO: 492 - protein, 13 aa, artificial sequence
  Scaffold Y protein
GAKKSKKRFSFKK

SEQ ID NO: 493 - protein, 12 aa, artificial sequence
  Scaffold Y protein
GAKKSKKRFSFK

SEQ ID NO: 494 - protein, 11 aa, artificial sequence
  Scaffold Y protein
GAKKSKKRFSF

SEQ ID NO: 495 - protein, 10 aa, artificial sequence
  Scaffold Y protein
GAKKSKKRFS

SEQ ID NO: 496 - protein, 9 aa, artificial sequence
  Scaffold Y protein
GAKKSKKRF

SEQ ID NO: 497 - protein, 8 aa, artificial sequence
  Scaffold Y protein
GAKKSKKR

SEQ ID NO: 498 - protein, 7 aa, artificial sequence
  Scaffold Y protein
GAKKSKK

SEQ ID NO: 499 - protein, 29 aa, artificial sequence
  Scaffold Y protein
GAKKAKKRFSFKKSFKLSGFSFKKNKKEA

SEQ ID NO: 500 - protein, 28 aa, artificial sequence
  Scaffold Y protein
GAKKAKKRFSFKKSFKLSGFSFKKNKKE

SEQ ID NO: 501 - protein, 27 aa, artificial sequence
  Scaffold Y protein
GAKKAKKRFSFKKSFKLSGFSFKKNKK

SEQ ID NO: 502 - protein, 26 aa, artificial sequence
  Scaffold Y protein
GAKKAKKRFSFKKSFKLSGFSFKKNK

SEQ ID NO: 503 - protein, 25 aa, artificial sequence
  Scaffold Y protein
GAKKAKKRFSFKKSFKLSGFSFKKN

SEQ ID NO: 504 - protein, 24 aa, artificial sequence
  Scaffold Y protein
GAKKAKKRFSFKKSFKLSGFSFKK

SEQ ID NO: 505 - protein, 23 aa, artificial sequence
  Scaffold Y protein
GAKKAKKRFSFKKSFKLSGFSFK

SEQ ID NO: 506 - protein, 22 aa, artificial sequence
  Scaffold Y protein
GAKKAKKRFSFKKSFKLSGFSF

SEQ ID NO: 507 - protein, 21 aa, artificial sequence
  Scaffold Y protein
GAKKAKKRFSFKKSFKLSGFS

SEQ ID NO: 508 - protein, 20 aa, artificial sequence
  Scaffold Y protein
GAKKAKKRFSFKKSFKLSGF

SEQ ID NO: 509 - protein, 19 aa, artificial sequence
  Scaffold Y protein
GAKKAKKRFSFKKSFKLSG

SEQ ID NO: 510 - protein, 18 aa, artificial sequence
  Scaffold Y protein
GAKKAKKRFSFKKSFKLS

SEQ ID NO: 511 - protein, 17 aa, artificial sequence
  Scaffold Y protein
GAKKAKKRFSFKKSFKL

SEQ ID NO: 512 - protein, 16 aa, artificial sequence
  Scaffold Y protein
GAKKAKKRFSFKKSFK

SEQ ID NO: 513 - protein, 15 aa, artificial sequence
  Scaffold Y protein
GAKKAKKRFSFKKSF

SEQ ID NO: 514 - protein, 14 aa, artificial sequence
  Scaffold Y protein
GAKKAKKRFSFKKS

SEQ ID NO: 515 - protein, 13 aa, artificial sequence
  Scaffold Y protein
GAKKAKKRFSFKK

SEQ ID NO: 516 - protein, 12 aa, artificial sequence
  Scaffold Y protein
GAKKAKKRFSFK

SEQ ID NO: 517 - protein, 11 aa, artificial sequence
  Scaffold Y protein
GAKKAKKRFSF

SEQ ID NO: 518 - protein, 10 aa, artificial sequence
  Scaffold Y protein
GAKKAKKRFS

SEQ ID NO: 519 - protein, 9 aa, artificial sequence
  Scaffold Y protein
GAKKAKKRF

SEQ ID NO: 520 - protein, 8 aa, artificial sequence
  Scaffold Y protein
GAKKAKKR

SEQ ID NO: 521 - protein, 7 aa, artificial sequence
  Scaffold Y protein
GAKKAKK

SEQ ID NO: 522 - protein, 28 aa, artificial sequence
  Scaffold Y protein
GAQESKKKKKKRFSFKKSFKLSGFSFKK

SEQ ID NO: 523 - protein, 27 aa, artificial sequence
  Scaffold Y protein
GAQESKKKKKKRFSFKKSFKLSGFSFK

SEQ ID NO: 524 - protein, 26 aa, artificial sequence
  Scaffold Y protein
GAQESKKKKKKRFSFKKSFKLSGFSF

SEQ ID NO: 525 - protein, 25 aa, artificial sequence
  Scaffold Y protein
GAQESKKKKKKRFSFKKSFKLSGFS

SEQ ID NO: 526 - protein, 24 aa, artificial sequence
  Scaffold Y protein
GAQESKKKKKKRFSFKKSFKLSGF

SEQ ID NO: 527 - protein, 23 aa, artificial sequence
  Scaffold Y protein
GAQESKKKKKKRFSFKKSFKLSG

SEQ ID NO: 528 - protein, 22 aa, artificial sequence
  Scaffold Y protein
GAQESKKKKKKRFSFKKSFKLS

SEQ ID NO: 529 - protein, 21 aa, artificial sequence
  Scaffold Y protein
GAQESKKKKKKRFSFKKSFKL

SEQ ID NO: 530 - protein, 20 aa, artificial sequence
  Scaffold Y protein
GAQESKKKKKKRFSFKKSFK

SEQ ID NO: 531 - protein, 19 aa, artificial sequence
  Scaffold Y protein
GAQESKKKKKKRFSFKKSF

SEQ ID NO: 532 - protein, 18 aa, artificial sequence
  Scaffold Y protein
GAQESKKKKKKRFSFKKS

SEQ ID NO: 533 - protein, 17 aa, artificial sequence
  Scaffold Y protein
GAQESKKKKKKRFSFKK

SEQ ID NO: 534 - protein, 16 aa, artificial sequence
  Scaffold Y protein
GAQESKKKKKKRFSFK

SEQ ID NO: 535 - protein, 15 aa, artificial sequence
  Scaffold Y protein
GAQESKKKKKKRFSF

SEQ ID NO: 536 - protein, 14 aa, artificial sequence
  Scaffold Y protein
GAQESKKKKKKRFS

SEQ ID NO: 537 - protein, 13 aa, artificial sequence
  Scaffold Y protein
GAQESKKKKKKRF

SEQ ID NO: 538 - protein, 12 aa, artificial sequence
  Scaffold Y protein
GAQESKKKKKKR

SEQ ID NO: 539 - protein, 11 aa, artificial sequence
  Scaffold Y protein
GAQESKKKKKK

SEQ ID NO: 540 - protein, 10 aa, artificial sequence
  Scaffold Y protein
GAQESKKKKK

SEQ ID NO: 541 - protein, 9 aa, artificial sequence
  Scaffold Y protein
GAQESKKKK

SEQ ID NO: 542 - protein, 8 aa, artificial sequence
  Scaffold Y protein
GAQESKKK

SEQ ID NO: 543 - protein, 7 aa, artificial sequence
  Scaffold Y protein
GAQESKK

SEQ ID NO: 544 - protein, 30 aa, artificial sequence
  Scaffold Y protein
GSQSSKKKKKKFSFKKPFKLSGLSFKRNRK

SEQ ID NO: 545 - protein, 29 aa, artificial sequence
  Scaffold Y protein
GSQSSKKKKKKFSFKKPFKLSGLSFKRNR

SEQ ID NO: 546 - protein, 28 aa, artificial sequence
  Scaffold Y protein
GSQSSKKKKKKFSFKKPFKLSGLSFKRN

SEQ ID NO: 547 - protein, 27 aa, artificial sequence
  Scaffold Y protein
GSQSSKKKKKKFSFKKPFKLSGLSFKR

SEQ ID NO: 548 - protein, 26 aa, artificial sequence
  Scaffold Y protein
GSQSSKKKKKKFSFKKPFKLSGLSFK

SEQ ID NO: 549 - protein, 25 aa, artificial sequence
  Scaffold Y protein
GSQSSKKKKKKFSFKKPFKLSGLSF

SEQ ID NO: 550 - protein, 24 aa, artificial sequence
  Scaffold Y protein
GSQSSKKKKKKFSFKKPFKLSGLS

SEQ ID NO: 551 - protein, 23 aa, artificial sequence
  Scaffold Y protein
GSQSSKKKKKKFSFKKPFKLSGL

SEQ ID NO: 552 - protein, 22 aa, artificial sequence
  Scaffold Y protein
GSQSSKKKKKKFSFKKPFKLSG

SEQ ID NO: 553 - protein, 21 aa, artificial sequence
  Scaffold Y protein
GSQSSKKKKKKFSFKKPFKLS

SEQ ID NO: 554 - protein, 20 aa, artificial sequence
  Scaffold Y protein
GSQSSKKKKKKFSFKKPFKL

SEQ ID NO: 555 - protein, 19 aa, artificial sequence
  Scaffold Y protein
GSQSSKKKKKKFSFKKPFK

SEQ ID NO: 556 - protein, 18 aa, artificial sequence
  Scaffold Y protein
GSQSSKKKKKKFSFKKPF

SEQ ID NO: 557 - protein, 17 aa, artificial sequence
  Scaffold Y protein
GSQSSKKKKKKFSFKKP

SEQ ID NO: 558 - protein, 16 aa, artificial sequence
  Scaffold Y protein
GSQSSKKKKKKFSFKK

SEQ ID NO: 559 - protein, 15 aa, artificial sequence
  Scaffold Y protein
GSQSSKKKKKKFSFK

SEQ ID NO: 560 - protein, 14 aa, artificial sequence
  Scaffold Y protein
GSQSSKKKKKKFSF

SEQ ID NO: 561 - protein, 13 aa, artificial sequence
  Scaffold Y protein
GSQSSKKKKKKFS

SEQ ID NO: 562 - protein, 12 aa, artificial sequence
  Scaffold Y protein
GSQSSKKKKKKF

SEQ ID NO: 563 - protein, 11 aa, artificial sequence
  Scaffold Y protein
GSQSSKKKKKK

SEQ ID NO: 564 - protein, 10 aa, artificial sequence
  Scaffold Y protein
GSQSSKKKKK

SEQ ID NO: 565 - protein, 9 aa, artificial sequence
  Scaffold Y protein
GSQSSKKKK

SEQ ID NO: 566 - protein, 8 aa, artificial sequence
  Scaffold Y protein
GSQSSKKK

SEQ ID NO: 567 - protein, 7 aa, artificial sequence
  Scaffold Y protein
GSQSSKK

SEQ ID NO: 568 - protein, 14 aa, artificial sequence
  Scaffold Y protein
GGKLSKKKSGGSGG

SEQ ID NO: 569 - protein, 14 aa, artificial sequence
  Scaffold Y protein
GGKLSKSGGSGGSV

(19) United States
(12) Patent Application Publication
(10) Pub. No.: US 2022/0395465 A1
(43) Pub. Date: Dec. 15, 2022

(54) STING AGONIST COMPRISING EXOSOMES COMBINED WITH IL-12 DISPLAYING EXOSOMES FOR TREATING A TUMOUR

(71) Applicant: CODIAK BIOSCIENCES, INC., Cambridge, MA (US)

(72) Inventors: Nuruddeen LEWIS, Andover, MA (US); Sriram SATHYANARAYANAN, Lexington, MA (US)

(73) Assignee: CODIAK BIOSCIENCES, INC., Cambridge, MA (US)

(21) Appl. No.: 17/763,973

(22) PCT Filed: Sep. 24, 2020
(86) PCT No.: PCT/US2020/052587

§ 371 Date: Mar. 25, 2022

Related U.S. Application Data
(60) Provisional application No. 63/070,149, filed on Aug. 25, 2020.
(60) Provisional application No. 63/066,605, filed on Aug. 17, 2020.
(60) Provisional application No. 62/906,016, filed on Sep. 25, 2019.

(51) Int. Cl. A61K 9/50; A61P 35/00; A61K 31/7084; A61K 38/20; A61K 39/395; A61K 47/69

(52) U.S. Cl. CPC A61K 9/5068; A61P 35/00; A61K 31/7084; A61K 38/208; A61K 39/39558; A61K 47/6901

(57) ABSTRACT

Provided herein are methods of treating a tumor comprising administering (i) a composition comprising an extracellular vesicle and a STING agonist, e.g., exosome encapsulating STING agonists, in combination with (ii) an IL-12 moiety.

### CROSS REFERENCE TO RELATED APPLICATIONS

This PCT application claims the priority benefit of U.S. Provisional Application Nos. 62/906,016 filed Sep. 25, 2019; 63/066,605 filed Aug. 17, 2020; and 63/070,149 filed Aug. 25, 2020; each of which is incorporated herein by reference in its entirety.

### REFERENCE TO SEQUENCE LISTING SUBMITTED ELECTRONICALLY VIA EFS-WEB

The content of the electronically submitted sequence listing in ASCII text file (Name: 4000_072PC03_Seglisting_ST25; Size: 101,647 bytes; and Date of Creation: Sep. 24, 2020), filed with the application, is incorporated herein by reference in its entirety.

### BACKGROUND OF THE DISCLOSURE

Stimulator of Interferon Genes (STING) is a cytosolic sensor of cyclic dinucleotides that is typically produced by bacteria. Upon activation, it leads to the production of type I interferons and initiates an immune response. Agonism of STING has been shown as a promising approach for generating an immune response against tumors pre-clinically. Unfortunately, given the broad expression profile of STING, systemic delivery of STING agonists leads to systemic inflammation. This limits the dose that can be given which in turn limits the therapeutic efficacy. An alternative approach to systemic delivery is to inject the STING agonist directly into the tumor. Intra-tumoral injections are quite effective; however, they are limited to solid tumors that can be reached with a needle and lead to tissue damage. Improved methods of delivering STING agonists are therefore needed.

### SUMMARY OF THE DISCLOSURE

Certain aspects of the present disclosure are directed to a method of treating a tumor in a subject in need thereof comprising administering (i) a composition comprising an extracellular vesicle (EV) and a stimulator of interferon genes protein (STING) agonist in combination with (ii) an interleukin 12 (IL-12) moiety. In some aspects, the IL-12 moiety is associated with a second EV. In some aspects, the IL-12 moiety is associated with the EV comprising the STING agonist.

In some aspects, the tumor is a primary tumor, a secondary tumor, or both a primary tumor and a secondary tumor.

In some aspects, the administering reduces the volume of the tumor.

In some aspects, the administering reduces the volume of the tumor by at least two fold, at least three fold, at least four fold, at least five fold, at least six fold, at least seven fold, at least nine fold, or at least ten fold compared to the tumor volume after administering either an extracellular vesicle comprising the STING agonist or the IL-12 moiety (“monotherapy”).

In some aspects, the administering reduces the volume of the primary tumor. In some aspects, the administering is capable of reducing the volume of the primary tumor by at least about 1.5 fold, at least about 2 fold, at least about 3 fold, at least about 4 fold, or at least about 5 fold compared to the monotherapy after day 14 of the administering.

In some aspects, the administering reduces the volume of the secondary tumor. In some aspects, the administering is capable of reducing the volume of the secondary tumor by at least about 1.5 fold, at least about 1.6 fold, at least about 1.7 fold, at least about 1.8 fold, at least about 1.9 fold, or at least about 2 fold compared to the monotherapy after day 14 of the administering. In some aspects, the administering reduces the growth of the tumor.

In some aspects, the administering reduces the growth of the tumor by at least two fold, at least three fold, at least four fold, at least five fold, at least six fold, at least seven fold, at least nine fold, or at least ten fold compared to the tumor volume after administering either an extracellular vesicle comprising the STING agonist or the IL-12 moiety (“monotherapy”). In some aspects, the administering reduces the growth of the primary tumor and/or the secondary tumor.

In some aspects, the method further comprises administering an anti-cancer agent. In some aspects, the anti-cancer agent comprises a checkpoint inhibitor. In some aspects, the checkpoint inhibitor comprises an anti-PD-1 antibody, an anti-PD-L1 antibody, an anti-CTLA-4 antibody, an anti-LAG-3 antibody, an anti-TIM-3 antibody, or any combination thereof. In some aspects, the checkpoint inhibitor is an anti-PD-1 antibody.

Certain aspects of the present disclosure are directed to an extracellular vesicle comprising a STING agonist and an IL-12 moiety.

Certain aspects of the present disclosure are directed to a composition comprising an extracellular vesicle comprising a STING agonist and a second EV comprising an IL-12 moiety.

In certain aspects, the IL-12 moiety is an IL-12 protein, a nucleic acid encoding an IL-12 protein, or a molecule having an IL-12 activity. In certain aspects, the IL-12 moiety is an IL-12 protein.

In certain aspects, the extracellular vesicle is an exosome, a nanovesicle, an apoptotic body, a microvesicle, a lysosome, an endosome, a liposome, a lipid nanoparticle, a micelle, a multilamellar structure, a revesiculated vesicle, or an extruded cell. In some aspects, the EV is an exosome.

In some aspects, the STING agonist is associated with the EV. In some aspects, the STING agonist is encapsulated within the EV. In some aspects, the STING agonist is linked to a lipid bilayer of the EV, optionally by a linker. In some aspects, the EV overexpresses a Prostaglandin F2 receptor negative regulator (PTGFRN) protein. In some aspects, the STING agonist is not linked to the PTGFRN protein. In some aspects, the extracellular vesicle is produced by a cell that overexpresses a PTGFRN protein.

In some aspects, the extracellular vesicle further comprises a ligand, a cytokine, or an antibody. In some aspects, the antibody comprises an antagonistic antibody and/or an agonistic antibody.

In some aspects, the STING agonist is a cyclic dinucleotide. In some aspects, the STING agonist is a non-cyclic dinucleotide. In some aspects, the STING agonist comprises a lipid-binding tag.

In some aspects, the STING agonist is physically and/or chemically modified. In some aspects, the modified STING agonist has a polarity and/or a charge different from the corresponding unmodified STING agonist.

In some aspects, the concentration of the STING agonist is about 0.01 μM to 100 μM. In some aspects, the concentration of the STING agonist is about 0.01 μM to 0.1 μM, 0.1 μM to 1 μM, 1 μM to 10 μM, 10 μM to 50 μM, or 50 μM to 100 μM. In some aspects, the concentration of the STING agonist in the EV is about 1 μM to 10 μM.

In some aspects, the STING agonist comprises:

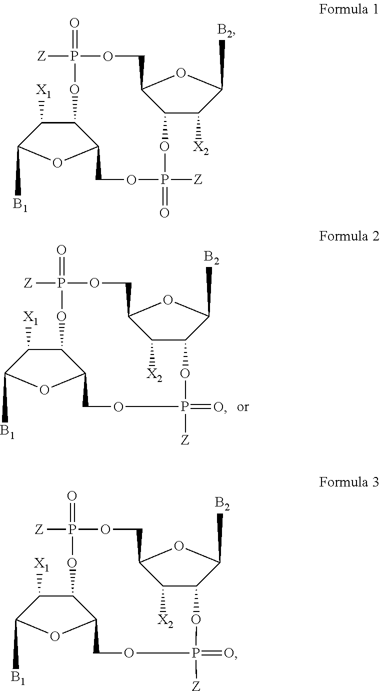

wherein:

X₁ is H, OH, or F;

X₂ is H, OH, or F;

Z is OH, OR₁, SH or SR₁, wherein:

R₁ is Na or NH₄, or

R₁ is an enzyme-labile group which provides OH or SH in vivo such as pivaloyloxymethyl;

Bi and B2 are bases chosen from:

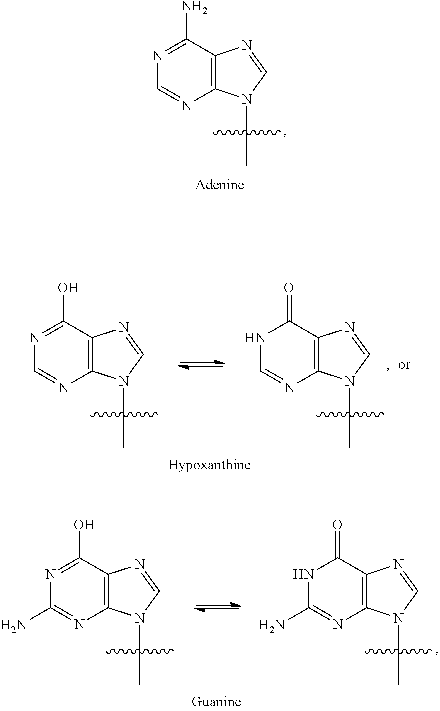 [SMILES: *n1cnc2c(N)ncnc21 ; *n1cnc2c(O)ncnc21 ; *n1cnc2c(=O)[nH]cnc21 ; *n1cnc2c(O)nc(N)nc21 ; *n1cnc2c(=O)[nH]c(N)nc21]

with the proviso that:
in Formula (I): X₁ and X₂ are not OH,
in Formula (II): when X₁ and X₂ are OH, B₁ is not Adenine and B₂ is not Guanine, and
in Formula (III): when X₁ and X₂ are OH, B₁ is not Adenine, B₂ is not Guanine and Z is not OH, or a pharmaceutically acceptable salt thereof.

In some aspects, the STING agonist is selected from the group consisting of:

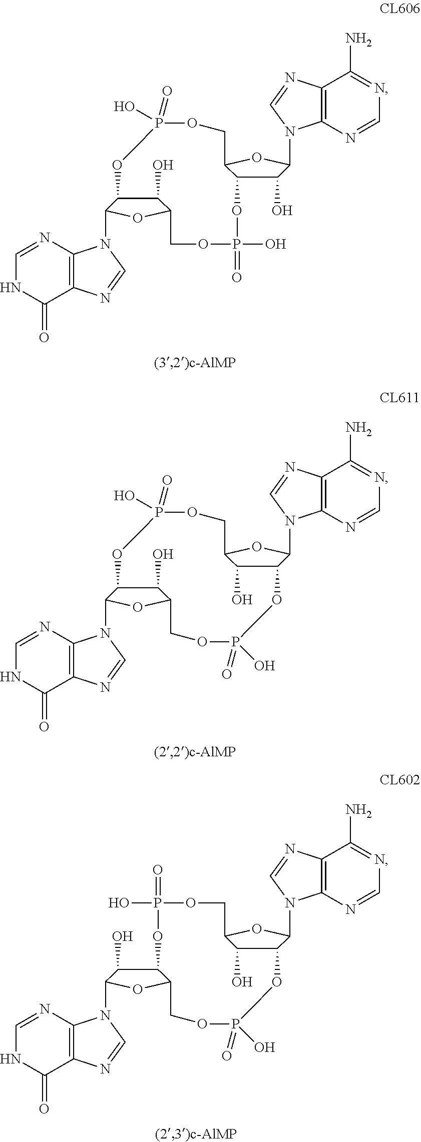 [SMILES: Nc1ncnc2c1ncn2C1OC2COP(=O)(O)O[C@H]3C(n4cnc5c(=O)[nH]cnc54)OC(COP(=O)(O)O[C@H]2[C@H]1O)[C@H]3O ; Nc1ncnc2c1ncn2C1OC2COP(=O)(O)O[C@H]3C(n4cnc5c(=O)[nH]cnc54)OC(COP(=O)(O)O[C@@H]1[C@@H]2O)[C@H]3O ; Nc1ncnc2c1ncn2C1OC2COP(=O)(O)O[C@@H]3C(COP(=O)(O)O[C@@H]1[C@@H]2O)OC(n1cnc2c(=O)[nH]cnc21)[C@@H]3O]

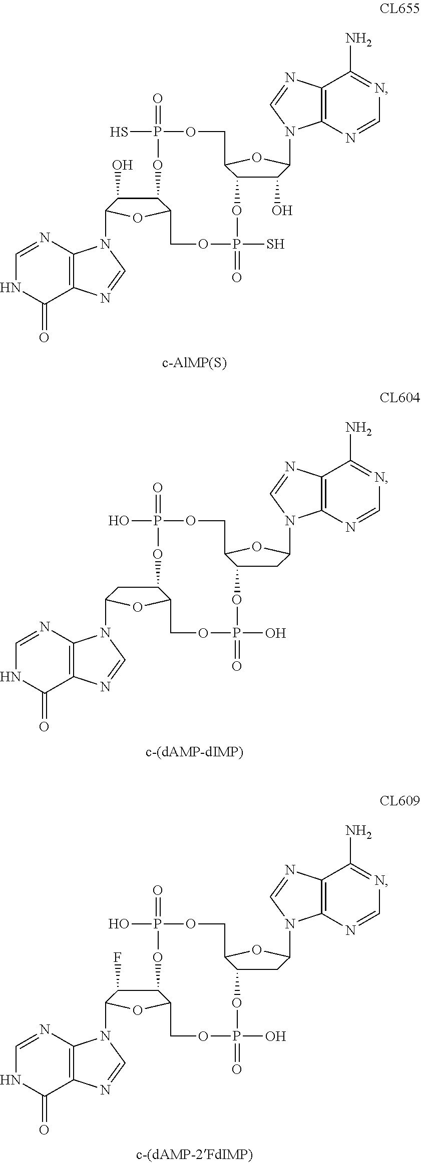 [SMILES: Nc1ncnc2c1ncn2C1OC2COP(=O)(S)O[C@@H]3C(COP(=O)(S)O[C@H]2[C@H]1O)OC(n1cnc2c(=O)[nH]cnc21)[C@@H]3O ; Nc1ncnc2c1ncn2C1C[C@@H]2OP(=O)(O)OCC3OC(n4cnc5c(=O)[nH]cnc54)C[C@@H]3OP(=O)(O)OCC2O1 ; Nc1ncnc2c1ncn2C1C[C@@H]2OP(=O)(O)OCC3OC(n4cnc5c(=O)[nH]cnc54)[C@H](F)[C@@H]3OP(=O)(O)OCC2O1]

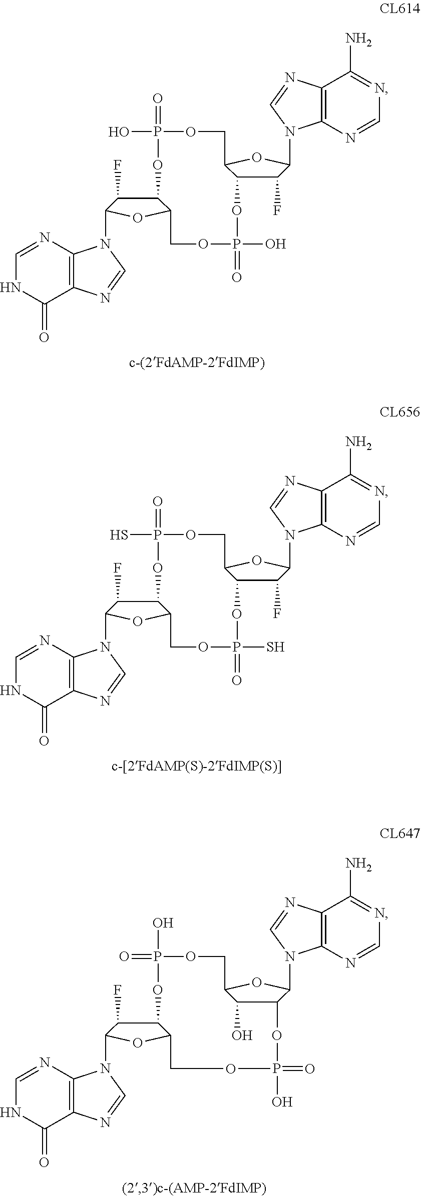 [SMILES: Nc1ncnc2c1ncn2C1OC2COP(=O)(O)O[C@@H]3C(COP(=O)(O)O[C@H]2[C@H]1F)OC(n1cnc2c(=O)[nH]cnc21)[C@@H]3F ; Nc1ncnc2c1ncn2C1OC2COP(=O)(S)O[C@@H]3C(COP(=O)(S)O[C@H]2[C@H]1F)OC(n1cnc2c(=O)[nH]cnc21)[C@@H]3F ; Nc1ncnc2c1ncn2C1OC2COP(=O)(O)O[C@@H]3C(COP(=O)(O)OC1[C@@H]2O)OC(n1cnc2c(=O)[nH]cnc21)[C@@H]3F]

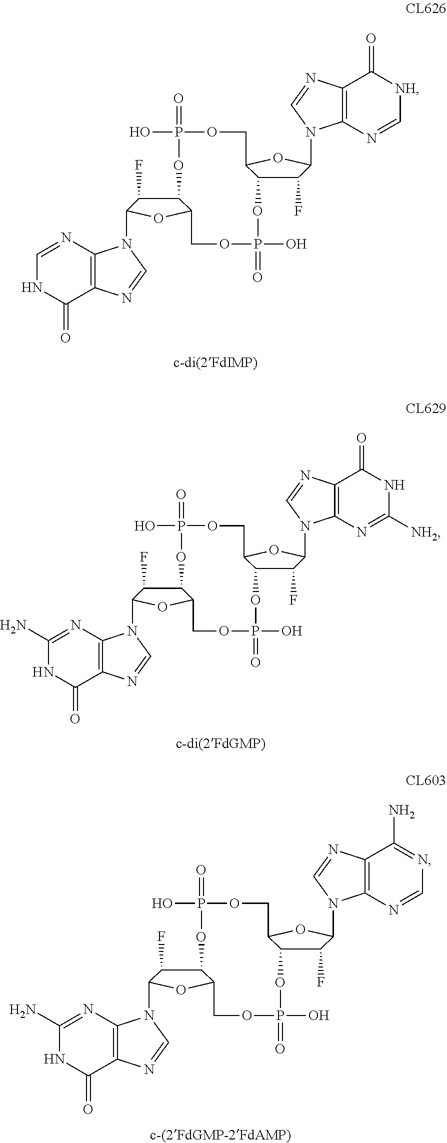 [SMILES: O=c1[nH]cnc2c1ncn2C1OC2COP(=O)(O)O[C@@H]3C(COP(=O)(O)O[C@H]2[C@H]1F)OC(n1cnc2c(=O)[nH]cnc21)[C@@H]3F ; Nc1nc2c(ncn2C2OC3COP(=O)(O)O[C@@H]4C(COP(=O)(O)O[C@H]3[C@H]2F)OC(n2cnc3c(=O)[nH]c(N)nc32)[C@@H]4F)c(=O)[nH]1 ; Nc1nc2c(ncn2C2OC3COP(=O)(O)O[C@@H]4C(COP(=O)(O)O[C@H]3[C@H]2F)OC(n2cnc3c(N)ncnc32)[C@@H]4F)c(=O)[nH]1]

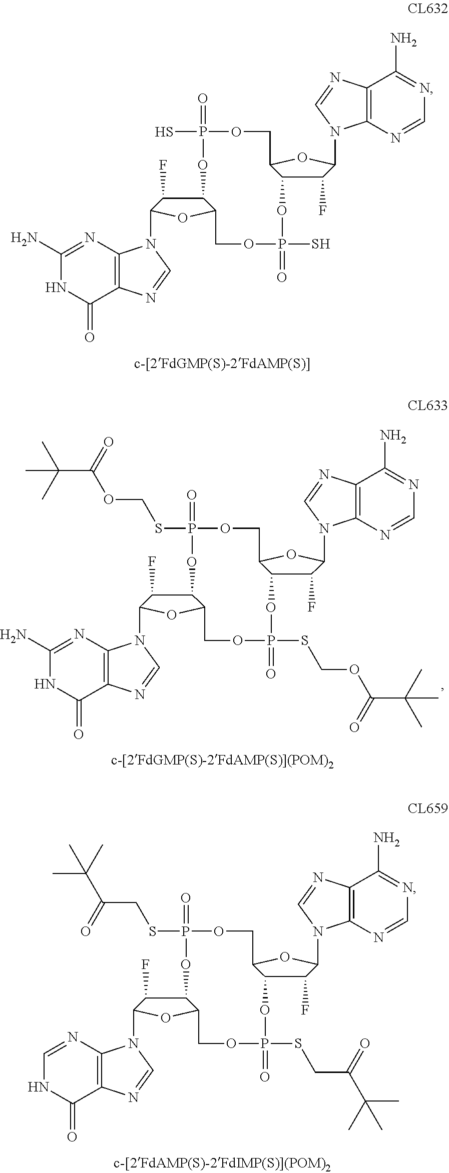 [SMILES: Nc1nc2c(ncn2C2OC3COP(=O)(S)O[C@@H]4C(COP(=O)(S)O[C@H]3[C@H]2F)OC(n2cnc3c(N)ncnc32)[C@@H]4F)c(=O)[nH]1 ; CC(C)(C)C(=O)OCSP1(=O)OCC2OC(n3cnc4c(=O)[nH]c(N)nc43)[C@H](F)[C@@H]2OP(=O)(SCOC(=O)C(C)(C)C)OCC2OC(n3cnc4c(N)ncnc43)[C@H](F)[C@@H]2O1 ; CC(C)(C)C(=O)CSP1(=O)OCC2OC(n3cnc4c(=O)[nH]cnc43)[C@H](F)[C@@H]2OP(=O)(SCC(=O)C(C)(C)C)OCC2OC(n3cnc4c(N)ncnc43)[C@H](F)[C@@H]2O1]

and a pharmaceutically acceptable salt thereof.

In some aspects, the IL-12 moiety is linked to a scaffold moiety. In some aspects, the second EV comprises a scaffold moiety. In some aspects, the IL-12 moiety is linked to the scaffold moiety. In some aspects, the scaffold moiety comprises a PTGFRN protein. In some aspects, the PTGFRN protein comprises SEQ ID NO: 33. In some aspects, the PTGFRN protein comprises at least about 70%, at least about 85%, at least about 90%, at least about 95%, at least about 96%, at least about 97%, at least about 98%, at least about 99%, or about 100% identical to SEQ ID NO: 1. In some aspects, the PTGFRN protein comprises the amino acid sequence as set forth in SEQ ID NO: 1.

In some aspects, the administration is parenterally, orally, intravenously, intramuscularly, intra-tumorally, intraperitoneally, or via any other appropriate administration route. In some aspects, the administration is intratumoral.

Certain aspects of the present disclosure are directed to a pharmaceutical composition comprising an EV disclosed herein or a composition disclosed herein and a pharmaceutically acceptable carrier.

Certain aspects of the present disclosure are directed to a kit comprising a composition disclosed herein and instructions for use.

### Aspects

### BRIEF DESCRIPTION OF THE DRAWINGS

FIG. 1 shows a diagram of the method of loading exosomes with a STING agonist.

FIGS. 2A-2B are graphical representations of average tumor volume (mm³) in B16F10 mice inoculated at a primary site (primary tumor; FIG. 2A) and a secondary site (secondary tumor;

FIG. 2B) following administration of PBS control (squares), exosomes with surface-display of IL-12 (“exoIL-12”) (inverted triangles), exosome-loaded STING agonists (“exoSTING”)(triangles), or a combination therapy of exosomes with surface-display of IL-12 and exosome-loaded STING agonists (circles). Error bars indicate standard error of the mean. FIG. 2C is a box-plot graph illustrating the growth rate of primary and secondary tumors in B16F10 mice following administration of PBS control (squares), exosomes with surface-display of IL-12 (inverted triangles), exosome-loaded STING agonists (triangles), or a combination therapy of exosomes with surface-display of IL-12 and exosome-loaded STING agonists (circles). Statistical significance was determined by a one-way ANOVA with a Tukey post-hoc (FIG. 2C). * p<0.05 vs PBS; *** p<0.001 vs PBS; #p<0.05 vs exoIL-12; ⊥ p<0.05 vs exoSTING; ⊥⊥⊥ p<0.001 vs exoSTING.

FIGS. 3A-3B are graphical representations of tumor volume in secondary (FIG. 3A) and primary (FIG. 3B) tumors following administration of in B16F10 mice following administration of PBS+IgG control (squares); exosome-loaded STING agonists+IgG (triangles); exosome-loaded STING agonists+ an antibody that binds programmed death 1 (anti-PD-1 antibody; inverted triangles); exosomes with surface-display of IL-12+IgG (open triangles); exosomes with surface-display of IL-12+ an anti-PD-1 antibody (open inverted triangles); a triple combination therapy of exosomes with surface-display of IL-12, exosome-loaded STING agonists, and IgG (circles); or a triple combination therapy of exosomes with surface-display of IL-12, exosome-loaded STING agonists, and an anti-PD-1 antibody (asterisk).

FIGS. 4A-4B are graphical representations of the growth rate of primary (FIG. 4A) and secondary (FIG. 4B) tumors in B16F10 mice following administration of PBS+IgG control; exosome-loaded STING agonists+IgG; exosome-loaded STING agonists+ an anti-PD-1 antibody; exosomes with surface-display of IL-12+IgG; exosomes with surface-display of IL-12+ an anti-PD-1 antibody; a triple combination therapy of exosomes with surface-display of IL-12, exosome-loaded STING agonists, and IgG; or a triple combination therapy of exosomes with surface-display of IL-12, exosome-loaded STING agonists, and an anti-PD-1 antibody (as indicated). Statistical significance was determined by a one-way ANOVA with a Tukey post-hoc (FIGS. 4A-4B). Lower case letters above each data set indicate statistical groups (FIG. 4A). * p<0.05; ** p<0.001; * ** p<0.0005; **** p<0.0001 (FIG. 4B).

FIGS. 5A-5N are line graphs representing the tumor volumes for each of the 5 mice treated in each group: primary tumor treated with PBS+IgG control (FIG. 5A); primary tumor treated with exosome-loaded STING agonists+IgG (FIG. 5B); primary tumor treated with exosome-loaded STING agonists+ an anti-PD-1 antibody (FIG. 5C); primary tumor treated with exosomes with surface-display of IL-12+IgG (FIG. 5D); primary tumor treated with exosomes with surface-display of IL-12+ an anti-PD-1 antibody (FIG. 5E); primary tumor treated with a triple combination therapy of exosomes with surface-display of IL-12, exosome-loaded STING agonists, and IgG (FIG. 5F); primary tumor treated with a triple combination therapy of exosomes with surface-display of IL-12, exosome-loaded STING agonists, and an anti-PD-1 antibody (FIG. 5G); secondary tumor following treatment with PBS+IgG control (FIG. 5H); secondary tumor following treatment with exosome-loaded STING agonists+IgG (FIG. 5I); secondary tumor following treatment with exosome-loaded STING agonists+ an anti-PD-1 antibody (FIG. 5J); secondary tumor following treatment with exosomes with surface-display of IL-12+IgG (FIG. 5K); secondary tumor following treatment with exosomes with surface-display of IL-12+ an anti-PD-1 antibody (FIG. 5L); secondary tumor following treatment with a triple combination therapy of exosomes with surface-display of IL-12, exosome-loaded STING agonists, and IgG (FIG. 5M); and secondary tumor following treatment with a triple combination therapy of exosomes with surface-display of IL-12, exosome-loaded STING agonists, and an anti-PD-1 antibody (FIG. 5N).

FIG. 6 is a drawing of an engineered exosome having surface displayed IL-12 (“exoIL-12”).

FIGS. 7A-7D are scatter plots showing tumor retention (FIG. 7A), IFN-gamma AUC (FIG. 7B), tumor growth (FIG. 7C), and tumor growth following rechallenge in untreated mice and mice treated with free recombinant IL-12 or exoIL-12, as indicated. FIG. 7E is a box-plot showing the percent of antigen-specific CD8+ T cells in control and exoIL-12-treated mice.

FIGS. 8A-8C are graphical representations the pharmacokinetics, as measured by exoIL-12 measured in the skin and plasma (FIG. 8A), and tissue pharmacodynamics, as measured by the fold change over vehicle of IP-10 in the skin (FIG. 8B) and plasma (FIG. 8C) over time, following administration of 0.3 μg, 1.0 μg, or 3.0 μg exoIL-12.

FIGS. 9A-9B are schematic representations of a clinical study assessing the safety and efficacy of exoIL-12 treatment in healthy volunteers (FIG. 9A) and cancer patients (FIG. 9B).

FIGS. 10A-10C are graphical representations of primary (FIG. 10B) and secondary (FIG. 10A) tumor volume (mm³) over up to 20 days and the number of CD8+ cells per mm² (FIG. 10C) following administration of (i) a combination of exosomes and IgG control, (ii) a combination of exosomes and an anti-PD-1 antibody, (iii) a combination of exosome-loaded STING agonists and an IgG control, (iv) a combination of exosome-loaded STING agonists and an anti-PD-1 antibody, (v) a combination of exoIL-12 and an anti-PD-1 antibody, (vi) a combination of exosome-loaded STING agonists and exoIL-12, and (vii) a combination of exosome-loaded STING agonists (10 ng) and exoIL-12 (10 ng), as indicated.

### DETAILED DESCRIPTION OF THE DISCLOSURE

Before the present invention is described in greater detail, it is to be understood that this invention is not limited to particular aspects described, as such can, of course, vary. It is also to be understood that the terminology used herein is for the purpose of describing particular aspects only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible.

### I. Definitions

It is noted that, as used herein and in the appended claims, the singular forms “a,” “an,” and “the” include plural referents unless the context clearly dictates otherwise. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, and U represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” or “approximately” is used herein to mean approximately roughly, around, or in the region of. When the term “about” is used in conjunction with a numerical range, it modifies that range by extending the boundaries above and below the numerical values set forth. The term used herein means within 5% of the referenced amount, e.g., about 50% is understood to encompass a range of values from 47.5% to 52.5%.

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. Generally extracellular vesicles range in diameter from 20 nm to 1000 nm, and can comprise various macromolecular payload either within the internal space (i.e., lumen), displayed on the external surface of the extracellular vesicle, and/or spanning the membrane. Said payload can comprise nucleic acids, proteins, carbohydrates, lipids, small molecules, and/or combinations thereof. In some aspects, an extracellular vesicle comprises a scaffold moiety. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, extracellular vesicles are produced by cells that express one or more transgene products.

As used herein the term “exosome” refers to a cell-derived small (between 20-300 nm in diameter, e.g., 40-200 nm in diameter) vesicle comprising a membrane that encloses an internal space (i.e., lumen), and which is generated from said cell by direct plasma membrane budding or by fusion of the late endosome with the plasma membrane. In some aspects, the EVs, e.g., exosomes, are about 20 nm to about 300 nm. The exosome is a species of extracellular vesicle. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. In some aspects, an exosome comprises a scaffold moiety. The exosome can be derived from a producer cell, and isolated from the producer cell based on its size, density, biochemical parameters, or a combination thereof. In some aspects, the exosomes of the present disclosure are produced by cells that express one or more transgene products.

As used herein, the term “nanovesicle” refers to a cell-derived small (between 20-250 nm in diameter, more preferably 30-150 nm in diameter) vesicle comprising a membrane that encloses an internal space, and which is generated from said cell by direct or indirect manipulation such that said nanovesicle would not be produced by said producer cell without said manipulation. Appropriate manipulations of said producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. In some aspects, a nanovesicle comprises a scaffold moiety. The nanovesicle, once it is derived from a producer cell according to said manipulation, may be isolated from the producer cell based on its size, density, biochemical parameters, or a combination thereof.

The term “modified,” when used in the context of exosomes described herein, refers to an alteration or engineering of an EV, such that the modified EV is different from a naturally-occurring EV. In some aspects, a modified EV described herein comprises a membrane that differs in composition of a protein, a lipid, a small molecular, a carbohydrate, etc. compared to the membrane of a naturally-occurring EV (e.g., membrane comprises higher density or number of natural EV proteins and/or membrane comprises proteins that are not naturally found in EVs. In certain aspects, such modifications to the membrane changes the exterior surface of the EV. In certain aspects, such modifications to the membrane changes the lumen of the EV.

As used herein, the term “scaffold moiety” refers to a molecule that can be used to anchor STING agonists disclosed herein, an IL-12 moiety, and/or any other compound of interest (e.g., payload) to the EV either on the luminal surface or on the exterior surface of the EV. In certain aspects, a scaffold moiety comprises a synthetic molecule. In some aspects, a scaffold moiety comprises a non-polypeptide moiety. In other aspects, a scaffold moiety comprises a lipid, carbohydrate, or protein that naturally exists in the EV. In some aspects, a scaffold moiety comprises a lipid, carbohydrate, or protein that does not naturally exist in the exosome. In certain aspects, a scaffold moiety is Scaffold X. In some aspects, a scaffold moiety is Scaffold Y. In further aspects, a scaffold moiety comprises both Scaffold X and Scaffold Y. In certain aspects, a scaffold moiety comprises Lamp-1, Lamp-2, CD13, CD86, Flotillin, Syntaxin-3, CD2, CD36, CD40, CD40L, CD41a, CD44, CD45, ICAM-1, Integrin alpha4, L1CAM, LFA-1, Mac-1 alpha and beta, Vti-1A and B, CD3 epsilon and zeta, CD9, CD18, CD37, CD53, CD63, CD81, CD82, CXCR4, FcR, GluR2/3, HLA-DM (MHC II), immunoglobulins, MHC-I or MHC-II components, TCR beta, tetraspanins, or combinations thereof.

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); and a class of ATP transporter proteins (“the ATP1A1 protein,” “the ATP1A2 protein,” “the ATP1A3 protein,” “the ATP1A4 protein,” “the ATP1B₃ protein,” “the ATP2B₁ protein,” “the ATP2B₂ protein,” “the ATP2B₃ protein,” “the ATP2B protein”). In some aspects, a Scaffold X protein can be a whole protein or a fragment thereof (e.g., functional fragment, e.g., the smallest fragment that is capable of anchoring another moiety on the exterior surface or on the luminal surface of the EV, e.g., exosome). In some aspects, a Scaffold X can anchor a moiety (e.g., STING agonist and/or an IL-12 moiety) to the external surface or the luminal surface of the EVs, e.g., exosomes.

As used herein, the term “Scaffold Y” refers to exosome proteins that were newly identified within the luminal surface of exosomes. See, e.g., International Publication No. WO/2019/099942, which is incorporated herein by reference in its entirety. Non-limiting examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety on the luminal surface of the EVs, e.g., exosomes). In some aspects, a Scaffold Y can anchor a moiety (e.g., a STING agonist and/or an IL-12 moiety) to the lumen of the EVs, e.g., exosomes.

As used herein, the term “fragment” of a protein (e.g., therapeutic protein, Scaffold X, or Scaffold Y) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface and/or on the exterior surface of the EV. Similarly, in certain aspects, a functional fragment of a Scaffold Y protein retains the ability to anchor a moiety on the luminal surface of the EV. Whether a fragment is a functional fragment can be assessed by any art known methods to determine the protein content of EVs including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein. In some aspects, a functional fragment of a Scaffold Y protein retains at least about 50%, at least about 60%, at least about 70%, at least about 80%, at least about 90% or at least about 100% of the ability, e.g., an ability to anchor another molecule, of the naturally occurring Scaffold Y protein.

As used herein, the term “variant” of a molecule (e.g., functional molecule, antigen, Scaffold X and/or Scaffold Y) refers to a molecule that shares certain structural and functional identities with another molecule upon comparison by a method known in the art. For example, a variant of a protein can include a substitution, insertion, deletion, frameshift or rearrangement in another protein.

In some aspects, a variant of a Scaffold X comprises a variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins. In some aspects, variants or variants of fragments of PTGFRN share at least about 70%, at least about 80%, at least about 85%, at least about 90%, at least about 95%, at least about 96%, at least about 97%, at least about 98%, or at least about 99% sequence identity with PTGFRN according to SEQ ID NO: 1 or with a functional fragment thereof.

In some aspects, a variant of a Scaffold Y comprises a variant having at least 70% identity to MARCKS, MARCKSL1, BASP1 or a fragment of MARCKS, MARCKSL1, or BASP1. In some aspects variants or variants of fragments of MARCKS share at least about 70%, at least about 80%, at least about 85%, at least about 90%, at least about 95%, at least about 96%, at least about 97%, at least about 98%, or at least about 99% sequence identity with MARCKS according to SEQ ID NO: 401 or with a functional fragment thereof. In some aspects variants or variants of fragments of MARCKSL1 share at least about 70%, at least about 80%, at least about 85%, at least about 90%, at least about 95%, at least about 96%, at least about 97%, at least about 98%, or at least about 99% sequence identity with MARCKSL1 according to SEQ ID NO: 402 or with a functional fragment thereof. In some aspects variants or variants of fragments of BASP1 share at least about 70%, at least about 80%, at least about 85%, at least about 90%, at least about 95%, at least about 96%, at least about 97%, at least about 98%, or at least about 99% sequence identity with BASP1 according to SEQ ID NO: 403 or with a functional fragment thereof. In some aspects, the variant or variant of a fragment of Scaffold Y protein retains the ability to be specifically targeted to the lumen of EVs. In some aspects, the Scaffold Y includes one or more mutations, e.g., conservative amino acid substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences may be accomplished using readily available software both for online use and for download. Suitable software programs are available from various sources, and for alignment of both protein and nucleotide sequences. One suitable program to determine percent sequence identity is bl2 seq, part of the BLAST suite of programs available from the U.S. government's National Center for Biotechnology Information BLAST web site (blast.ncbi.nlm.nih.gov). Bl2 seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity may be curated either automatically or manually.

The polynucleotide variants can contain alterations in the coding regions, non-coding regions, or both. In one aspect, the polynucleotide variants contain alterations which produce silent substitutions, additions, or deletions, but do not alter the properties or activities of the encoded polypeptide. In another aspect, nucleotide variants are produced by silent substitutions due to the degeneracy of the genetic code. In other aspects, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X and/or Scaffold Y is modified at any convenient location.

As used herein the term “producer cell” refers to a cell used for generating an EV. A producer cell can be a cell cultured in vitro, or a cell in vivo. A producer cell includes, but not limited to, a cell known to be effective in generating EVs, e.g., exosomes, e.g., HEK293 cells, Chinese hamster ovary (CHO) cells, mesenchymal stem cells (MSCs), BJ human foreskin fibroblast cells, s9f cells, fHDF fibroblast cells, AGE.HN® neuronal precursor cells, CAP® amniocyte cells, adipose mesenchymal stem cells, and RPTEC/TERT1 cells. In certain aspects, a producer cell is an antigen-presenting cell. In some aspects, the producer cell is a bacterial cell. In some aspects, a producer cell is a dendritic cell, a B cell, a mast cell, a macrophage, a neutrophil, a Kupffer-Browicz cell, or a cell derived from any of these cells, or any combination thereof. In some aspects, the producer cell is not a bacterial cell. In other aspects, the producer cell is not an antigen-presenting cell.

As used herein the term “associated with” refers to encapsulation of a first moiety, e.g., a STING agonist and/or an IL-12 moiety, into a second moiety, e.g., extracellular vesicle, or to a covalent or non-covalent bond formed between a first moiety, e.g., a STING agonist (and/or an IL-12 moiety) and a second moiety, e.g., extracellular vesicle, respectively. For example, in some aspects, a scaffold moiety, e.g., Scaffold X (e.g., a PTGFRN protein), is expressed in or on the extracellular vesicle and a STING agonist, is loaded in the lumen of or on the external surface of the extracellular vesicle. For example, in some aspects, a scaffold moiety, e.g., Scaffold X (e.g., a PTGFRN protein), is expressed in or on the extracellular vesicle and an IL-12 moeity, is loaded on the external surface of the extracellular vesicle. In one aspect, the term “associated with” means a covalent, non-peptide bond or a non-covalent bond. For example, the amino acid cysteine comprises a thiol group that can form a disulfide bond or bridge with a thiol group on a second cysteine residue. Examples of covalent bonds include, but are not limited to, a peptide bond, a metal bond, a hydrogen bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, a hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity. In other aspects, the term “associated with” means that a first moiety, e.g., extracellular vesicle, encapsulates a second moiety, e.g., a STING agonist and/or an IL-12 moiety. In some aspects, the first moiety and the second moiety can be linked to each other. In other aspects, the first moiety and the second moiety are not physically and/or chemically linked to each other.

As used herein the term “linked to” or “conjugated to” are used interchangeably and refer to a covalent or non-covalent bond formed between a first moiety and a second moiety, e.g., a STING agonist and an extracellular vesicle and/or an IL-12 moiety and an extracellular vesicle, respectively. In some aspects, a scaffold moiety is expressed in or on the extracellular vesicle, e.g., Scaffold X (e.g., a PTGFRN protein), and an IL-12 moiety is linked to or conjugated to the portion of the Scaffold X protein (e.g., the PTGFRN protein) that is exposed on the surface of the extracellular vesicle (e.g., “surface-display of IL-12”). In some aspects, a scaffold moiety is expressed in or on the extracellular vesicle, e.g., Scaffold X (e.g., a PTGFRN protein), and a STING agonist and/or an IL-12 moiety is linked to or conjugated to the portion of the Scaffold X protein (e.g., the PTGFRN protein) that is exposed to the lumen of the extracellular vesicle.

The term “loaded”, or grammatically different forms of the term (e.g., load or loaded), as used herein, refers to a status or process of having a first moiety (e.g., a STING agonist and/or an IL-12 moiety) associated with a second moiety (e.g., an EV, e.g., and exosome). In some aspects, the first moiety is chemically or physically linked to the second moiety. In some aspects, the first moiety is not chemically or physically linked to the second moiety. In some aspects, the first moiety is present within the second moiety, e.g., within the lumen of an EV (e.g., an exosome), e.g., “encapsulated”. In some aspects, the first moiety is associated with the exterior surface of the second moiety, e.g., linked or conjugated to the surface of an EV (e.g., an exosome), e.g., “surface-display” of the second moiety.

The term “encapsulated”, or grammatically different forms of the term (e.g., encapsulation, or encapsulating), refers to a status or process of having a first moiety (e.g., a STING agonist and/or an IL-12 moiety) inside a second moiety (e.g., an EV, e.g., exosome) without chemically or physically linking the two moieties. In some aspects, the term “encapsulated” can be used interchangeably with “in the lumen of”. Non-limiting examples of encapsulating a first moiety (e.g., a STING agonist and/or an IL-12 moiety) into a second moiety (e.g., EVs, e.g., exosomes) are disclosed elsewhere herein.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, the term “agonist” refers to a molecule that binds to a receptor and activates the receptor to produce a biological response. Receptors can be activated by either an endogenous or an exogenous agonist. Non-limiting examples of endogenous agonist include hormones, neurotransmitters, and cyclic dinucleotides. Non-limiting examples of exogenous agonist include drugs, small molecules, and cyclic dinucleotides. The agonist can be a full, partial, or inverse agonist.

As used herein, the term “antagonist” refers to a molecule that blocks or dampens an agonist mediated response rather than provoking a biological response itself upon bind to a receptor. Many antagonists achieve their potency by competing with endogenous ligands or substrates at structurally defined binding sites on the receptors. Non-limiting examples of antagonists include alpha blockers, beta-blocker, and calcium channel blockers. The antagonist can be a competitive, non-competitive, or uncompetitive antagonist.

The term “free STING agonist” or “free IL-12 moiety” as used herein means a STING agonist or an IL-12 moiety that is not associated with an extracellular vesicle, but otherwise identical to the STING agonist or IL-12 moiety associated with the extracellular vesicle. Especially when compared to an extracellular vesicle associated with a STING agonist or an IL-12 moiety, the free STING agonist or the free IL-12 moiety is the same STING agonist or IL-12 moiety associated with the extracellular vesicle. In some aspects, when a free STING agonist is compared to an extracellular vesicle comprising the STING agonist in its efficacy, toxicity, and/or any other characteristics, the amount of the free STING agonist compared to the STING agonist associated with the extracellular vesicle is the same as the amount of the STING agonist associated with the EV. In some aspects, when a free IL-12 moiety is compared to an extracellular vesicle comprising the IL-12 moiety in its efficacy, toxicity, and/or any other characteristics, the amount of the free IL-12 moiety compared to the IL-12 moiety associated with the extracellular vesicle is the same as the amount of the IL-12 moiety associated with the EV.

The term “exoSTING” as used herein refers to an exosome loaded with a STING agonist. In some aspects, the exosome comprises STING agonist in the lumen of the exosome. In some aspects, the STING agonist is associated with the luminal surface of the exosome, e.g., with a Scaffold protein, e.g., Scaffold X, e.g., PTGFRN. In some aspects, the STING agonist is encapsulated within the lumen of the exosome and is not associated with a scaffold protein. In some aspects, the exosome comprises the STING agonist on the surface of the exosome. In some aspects, the STING agonist is associated with the exterior surface of the exosome. In some aspects, the STING agonist is linked to or conjugated to the exterior surface of the exosome. In some aspects, the STING agonist is linked to or conjugated to a surface exposed scaffold protein, e.g, a Scaffold X protein, e.g., a PTGFRN protein. In some aspects, the STING agonist is linked to or conjugated to the lipid bilayer of the exosome.

The term “exoIL-12” as used herein refers to an exosome loaded with an IL-12 moiety, e.g., an IL-12 protein or a fragment thereof. In some aspects, the IL-12 moiety is associated with the exterior surface of the exosome (e.g., surface display of the IL-12 moiety). Non-limiting examples of exosomes comprising an IL-12 moiety can be found, for example, in U.S. Pat. No. 10,723,782 and International Publication No. WO 2019/133934 A2, each of which is incorporated by reference herein in its entirety. In some aspects, the IL-12 moiety is linked to or conjugated to the exterior surface of the exosome. In some aspects, the IL-12 moiety is linked to or conjugated to a surface exposed scaffold protein, e.g, a Scaffold X protein, e.g., a PTGFRN protein. In some aspects, the IL-12 moiety is linked to or conjugated to the lipid bilayer of the exosome. In some aspects, the exosome comprises an IL-12 moiety in the lumen of the exosome. In some aspects, the IL-12 moiety is associated with the luminal surface of the exosome, e.g., with a Scaffold protein, e.g., Scaffold X, e.g., PTGFRN. In some aspects, the IL-12 moiety is encapsulated within the lumen of the exosome and is not associated with a scaffold protein.

As used herein, the term “ligand” refers to a molecule that binds to a receptor and modulates the receptor to produce a biological response. Modulation can be activation, deactivation, blocking, or damping of the biological response mediated by the receptor. Receptors can be modulated by either an endogenous or an exogenous ligand. Non-limiting examples of endogenous ligands include antibodies and peptides. Non-limiting examples of exogenous agonist include drugs, small molecules, and cyclic dinucleotides. The ligand can be a full, partial, or inverse ligand.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function.

As used herein the term “therapeutically effective amount” is the amount of reagent or pharmaceutical compound that is sufficient to a produce a desired therapeutic effect, pharmacologic and/or physiologic effect on a subject in need thereof. A therapeutically effective amount can be a “prophylactically effective amount” as prophylaxis can be considered therapy.

As used herein, the term “pharmaceutical compositio” refers to one or more of the compounds described herein, such as, e.g., an EV mixed or intermingled with, or suspended in one or more other chemical components, such as pharmaceutically-acceptable carriers and excipients. One purpose of a pharmaceutical composition is to facilitate administration of preparations of EVs to a subject. The term “excipient” or “carrier” refers to an inert substance added to a pharmaceutical composition to further facilitate administration of a compound. The term “pharmaceutically-acceptable carrier” or “pharmaceutically-acceptable excipient” and grammatical variations thereof, encompasses any of the agents approved by a regulatory agency of the US Federal government or listed in the US Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “payload” refers to a therapeutic agent that acts on a target (e.g., a target cell) that is contacted with the EV. Payloads that can be introduced into an EV and/or a producer cell include therapeutic agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, and small molecules (e.g., small molecule drugs and toxins).

The terms “administration,” “administering” and variants thereof refer to introducing a composition, such as an EV, or agent into a subject and includes concurrent and sequential introduction of a composition or agent. The introduction of a composition or agent into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

The term “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

The term “prevent” or “preventing,” as used herein, refers to decreasing or reducing the occurrence or severity of a particular outcome. In some aspects, preventing an outcome is achieved through prophylactic treatment.

As used herein, the term “modulate,” “modulating”, “modify,” and/or “modulator” generally refers to the ability to alter, by increase or decrease, e.g., directly or indirectly promoting/stimulating/up-regulating or interfering with/inhibiting/down-regulating a specific concentration, level, expression, function or behavior, such as, e.g., to act as an antagonist or agonist. In some instances a modulator can increase and/or decrease a certain concentration, level, activity or function relative to a control, or relative to the average level of activity that would generally be expected or relative to a control level of activity.

As used herein, “a mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

The terms “individual,” “subject,” “host,” and “patient,” are used interchangeably herein and refer to any mammalian subject for whom diagnosis, treatment, or therapy is desired, particularly humans. The methods described herein are applicable to both human therapy and veterinary applications. In some aspects, the subject is a mammal, and in other aspects the subject is a human.

As used herein, the term “substantially free” means that the sample comprising EVs comprise less than 10% of macromolecules by mass/volume (m/v) percentage concentration. Some fractions may contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein, the term “macromolecule” means nucleic acids, exogenous proteins, lipids, carbohydrates, metabolites, or a combination thereof.

As used herein, the term “insubstantial,” “reduced,” or “negligible” refers to the presence, level, or amount of an inflammation response in a subject after administration of the sample comprising EVs encapsulating a STING agonist relative to the baseline inflammation response in the subject or compared to the subject inflammation response to the administration of a free STING agonist. For example, a negligible or insubstantial presence, level or amount of systemic inflammation may be less than 0.001%, less than 0.01%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, less than 10%, less than 12%, less than 15%, less than 17%, less than 20%, or less than 25% of systemic inflammation as relative to the baseline inflammation in the subject or compared to the subject immune response to the administration of a free STING agonist. A level or amount of a systemic inflammation may be less than 0.1-fold, less than 0.5-fold, less than 0.5-fold, less than 1-fold, less than 1.5-fold, less than 2-fold relative to the baseline or compared to the inflammation response to the administration of a free STING agonist.

A “primary tumor,” as used herein, refers to an original, or first, tumor in a subject, where the tumor initiated growth. A primary tumor is used in contrast to a “secondary tumor,” which refers to a tumor that arises after initiation of growth of the primary tumor at a location other than the location of the primary tumor, e.g., due to metastasis of cells in the primary tumor.

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### II. Methods of Treating

Certain aspects of the present disclosure are directed to a method of treating a disease or condition, e.g., a tumor, in a subject in need thereof comprising administering (i) a composition comprising an extracellular vesicle (EV) and a stimulator of interferon genes protein (STING) agonistin combination with (ii) an interleukin 12 (IL-12) moiety. In some aspects, the IL-12 moiety is associated with an EV. In some aspects, the IL-12 moiety is associated with a second EV. In some aspects, the method comprises administering (i) a composition comprising a first EV and a STING agonist and (ii) a second EV associated with an IL-12 moiety. In some aspects, the IL-12 moiety is associated with the EV comprising the STING agonist, e.g., the IL-12 and the STING agonist are associated with the EV. In some aspects, the method comprises administering (i) a composition comprising an EV and a STING agonist, wherein the EV is associated with an IL-12 moiety. In some aspects, the EV is associated with the STING agonist.

In some aspects, the STING agonist is associated with the EV. In some aspects, the STING agonist is loaded within the lumen of the EV. In some aspects, the STING agonist is not associated with the EV. In some aspects, the STING agonist is loaded in a nanoparticle. In some aspects, the STING agonist is loaded in a nanoparticle selected from the group consisting of a lipid nanoparticle, a liposome, a polymeric micelle, a dendrimer, chitosan nanoparticle, an alginate nanoparticle, a xanthan gum-based nano-particle, a cellulose nanocrystal, an inorganic nanoparticle (e.g., silver, gold, iron oxide, and silica nanoparticles), a nanocrystal, a metallic nanoparticle, a quantum dot, and any combination thereof.

In some aspects, the tumor treatable by the present methods is a primary tumor, a secondary tumor, or both a primary tumor and a secondary tumor. In some aspects, the administering reduces the volume of the tumor. In some aspects, the administering reduces the volume of the tumor by at least about 2 fold, at least about 3 fold, at least about 4 fold, at least about 5 fold, at least about 6 fold, at least about 7 fold, at least about 8 fold, at least about 9 fold, or at least about 10 fold compared to the tumor volume after administering either an extracellular vesicle comprising the STING agonist or the IL-12 moiety (“monotherapy”).

In some aspects, the administering reduces the volume of a primary tumor. In some aspects, the administering is capable of reducing the volume of a primary tumor by at least about 1.5 fold, at least about 2 fold, at least about 3 fold, at least about 4 fold, at least about 5 fold, at least about 6 fold, at least about 7 fold, at least about 8 fold, at least about 9 fold, or at least about 10 fold compared to a monotherapy after day 14 of the administering.

In some aspects, the administering reduces the rate of tumor growth of a primary tumor. In some aspects, the administering is capable of reducing the growth rate of a primary tumor by at least about 1.5 fold, at least about 2 fold, at least about 3 fold, at least about 4 fold, at least about 5 fold, at least about 6 fold, at least about 7 fold, at least about 8 fold, at least about 9 fold, or at least about 10 fold compared to a monotherapy after day 14 of the administering. In some aspects, the administering is capable of ablating or stopping primary tumor growth.

The method of any one of claims 4 to 8, wherein the administering reduces the volume of a secondary tumor. In some aspects, the administering is capable of reducing the volume of a secondary tumor by at least about 1.5 fold, at least about 2 fold, at least about 3 fold, at least about 4 fold, at least about 5 fold, at least about 6 fold, at least about 7 fold, at least about 8 fold, at least about 9 fold, or at least about 10 fold compared to a monotherapy after day 14 of the administering.

In some aspects, the administering reduces the rate of tumor growth of a secondary tumor. In some aspects, the administering is capable of reducing the growth rate of a secondary tumor by at least about 1.5 fold, at least about 2 fold, at least about 3 fold, at least about 4 fold, at least about 5 fold, at least about 6 fold, at least about 7 fold, at least about 8 fold, at least about 9 fold, or at least about 10 fold compared to a monotherapy after day 14 of the administering. In some aspects, the administering is capable of ablating or stopping secondary tumor growth.

Other aspects of the disclosure are directed to methods of treating a disease or condition, e.g., a tumor, in a subject in need thereof comprising administering an extracellular vesicle (EV) comprising an interleukin 12 (IL-12) moiety, wherein the method does not comprise administering an EV comprising a STING agonist.

In some aspects, the method further comprises an anti-cancer agent. In some aspects, the anti-cancer agent comprises a checkpoint inhibitor. In some aspects, the checkpoint inhibitor comprises an anti-PD-1 antibody, an anti-PD-L1 antibody, an anti-CTLA-4 antibody, an anti-LAG-3 antibody, an anti-TIM-3 antibody, or any combination thereof. In certain aspects, the checkpoint inhibitor is an anti-PD-1 antibody.

### III. Compositions (Extracellular Vesicles)

Certain aspects of the present disclosure are directed to extracellular vesicles (EVs) comprising a STING agonist and/or an IL-12 moiety. In certain aspects, the EV comprises a STING agonist. In some aspects, the EV comprises an IL-12 moiety. In certain aspects, the EV comprises a STING agonist and an IL-12 moiety.

Some aspects of the present disclosure are directed to a composition comprising a first EV (e.g., exosome) and a second EV (e.g., exosome); wherein the first EV (e.g., exosome) comprises, e.g., is loaded with, a STING agonist; and the second EV (e.g., exosome) comprises, e.g., is loaded with, an IL-12 moiety. In some aspects, the STING agonist is encapsulated by the first EV (e.g., exosome). In some aspects, the IL-12 moiety is associated with the exterior surface of the second EV (e.g., exosome). In some aspects, the second EV (e.g., exosome) comprises surface-exposed IL-12.

In some aspects, the EV is an exosome, a nanovesicle, an apoptotic body, a microvesicle, a lysosome, an endosome, a liposome, a lipid nanoparticle, a micelle, a multilamellar structure, a revesiculated vesicle, or an extruded cell. In certain aspects, the EV is an exosome.

### III.A. STING Agonists

The innate immune system recognizes pathogen associated molecular patterns (PAMPs) via pattern recognition receptors (PRRs) that induce an immune response. PRRs recognize a variety of pathogen molecules including single and double stranded RNA and DNA. PRRS such as retinoic acid-inducible gene-I (RIG-I)-like receptors (RLRs) and some toll-like receptors (TLRs) recognize RNA ligands. DNA ligands are recognized by cyclic GMP-AMP synthase (cGAS), AIM2 and other TLRs. The TLRs, RLRs, and AIM2 directly interact with other signal cascade adaptor proteins to activate transcription factors, while cGAS produces cGAMP, a cyclic dinucleotide molecule that activates the stimulator of interferon gene (STING) receptor. Both STING and the RLRs activate the adaptor kinase TBK1 which induces activation of transcription factors IRF3, and NF-κB, and result in the production of type I IFNs and pro-inflammatory cytokines.

Cyclic dinucleotides (CDNs) were first identified as bacterial signaling molecules characterized by two 3′, 5′ phosphodiester bonds, such as in the molecule c-di-GMP. While STING can be activated by bacterial CDNs, the innate immune response in mammalian cells is also mediated by the CDN signaling molecule cGAMP which is produced by cGAS. cGAMP is characterized by a mixed 2′, 5′ and 3′, 5′ phosphodiester linkage. Both bacterial and mammalian CDNs directly interact with STING to induce the pro-inflammatory signaling cascade that results in the production of type I IFNs, such as IFNα and IFN-β.

STING agonists used in this disclosure can be cyclic dinucleotides (CDNs) or non-cyclic dinucleotide agonists. Cyclic purine dinucleotides such as, but not limited to, cGMP, cyclic di-GMP (c-di-GMP), cAMP, cyclic di-AMP (c-di-AMP), cyclic-GMP-AMP (cGAMP), cyclic di-IMP (c-di-IMP), cyclic AMP-IMP (cAIMP), and any analogue thereof, are known to stimulate or enhance an immune or inflammation response in a patient. The CDNs may have 2′2′, 2′3′, 2′5′, 3′3′, or 3′5′ bonds linking the cyclic dinucleotides, or any combination thereof.

Cyclic purine dinucleotides may be modified via standard organic chemistry techniques to produce analogues of purine dinucleotides. Suitable purine dinucleotides include, but are not limited to, adenine, guanine, inosine, hypoxanthine, xanthine, isoguanine, or any other appropriate purine dinucleotide known in the art. The cyclic dinucleotides may be modified analogues. Any suitable modification known in the art may be used, including, but not limited to, phosphorothioate, biphosphorothioate, fluorinate, and difluorinate modifications.

Non cyclic dinucleotide agonists may also be used, such as 5,6-Dimethylxanthenone-4-acetic acid (DMXAA), or any other non-cyclic dinucleotide agonist known in the art.

It is contemplated that any STING agonist may be used. Among the STING agonists are DMXAA, STING agonist-1, ML RR-S2 CDA, ML RR-S2c-di-GMP, ML-RR-S2 cGAMP, 2′3′-c-di-AM(PS)2, 2′3′-cGAMP, 2′3′-cGAMPdFHS, 3′3′-cGAMP, 3′3′-cGAMPdFSH, cAIMP, cAIM(PS)2, 3′3′-cAIMP, 3′3′-cAIMPdFSH, 2′2′-cGAMP, 2′3′-cGAM(PS)2, 3′3′-cGAMP, c-di-AMP, 2′3′-c-di-AMP, 2′3′-c-di-AM(PS)2, c-di-GMP, 2′3′-c-di-GMP, c-di-IMP, c-di-UMP or any combination thereof. In a preferred aspect, the STING agonist is 3′3′-cAIMPdFSH, alternatively named 3-3 cAIMPdFSH. Additional STING agonists known in the art may also be used.

In some aspects, the STING agonist useful for the present disclosure comprises a compound having the following formula:

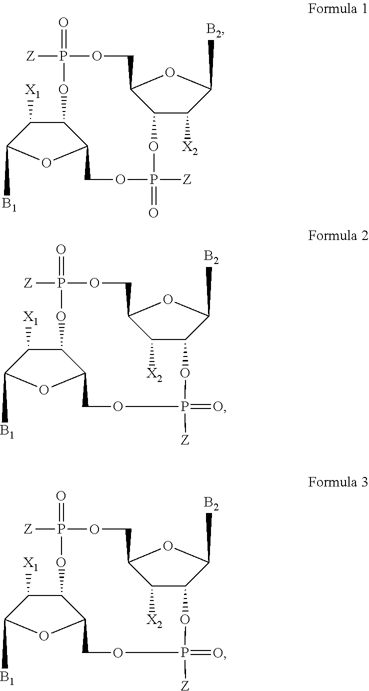

wherein:

### X₁ is H, OH, or F;

### X₂ is H, OH, or F;

Z is OH, OR₁, SH or SR₁, wherein:
i) R₁ is Na or NH₄, or
ii) R₁ is an enzyme-labile group which provides OH or SH in vivo such as pivaloyloxymethyl;
Bi and B₂ are bases chosen from:

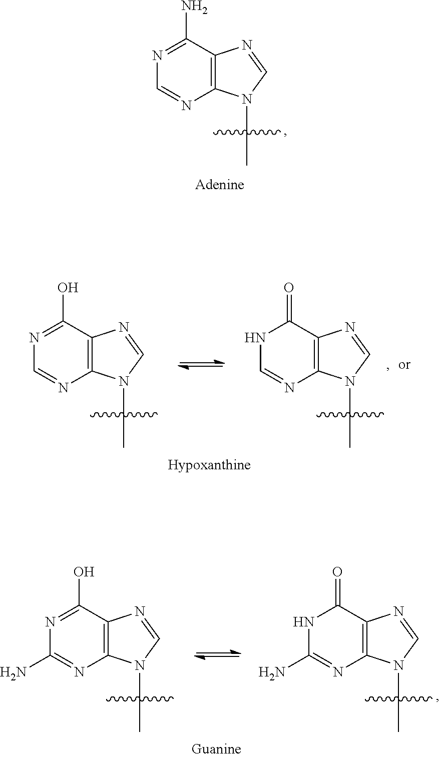 [SMILES: *n1cnc2c(N)ncnc21 ; *n1cnc2c(O)ncnc21 ; *n1cnc2c(=O)[nH]cnc21 ; *n1cnc2c(O)nc(N)nc21 ; *n1cnc2c(=O)[nH]c(N)nc21]

With proviso that:
in Formula (I): X₁ and X₂ are not OH,
in Formula (II): when X₁ and X₂ are OH, B₁ is not Adenine and B₂ is not Guanine, and
in Formula (III): when X₁ and X₂ are OH, B₁ is not Adenine, B₂ is not Guanine and Z is not OH.
   See WO 2016/096174, the content of which is incorporated herein by reference in its entirety.

In some aspects, the STING agonist useful for the present disclosure comprises:

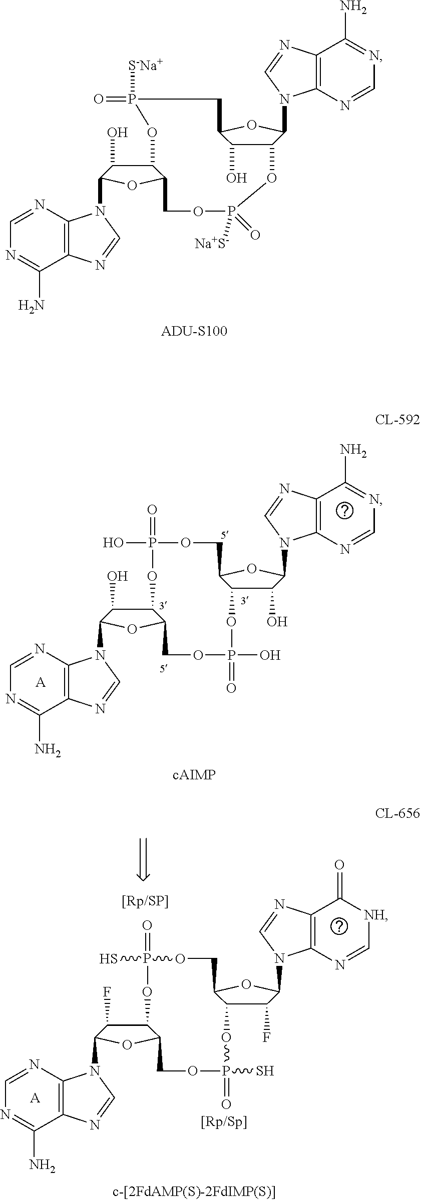

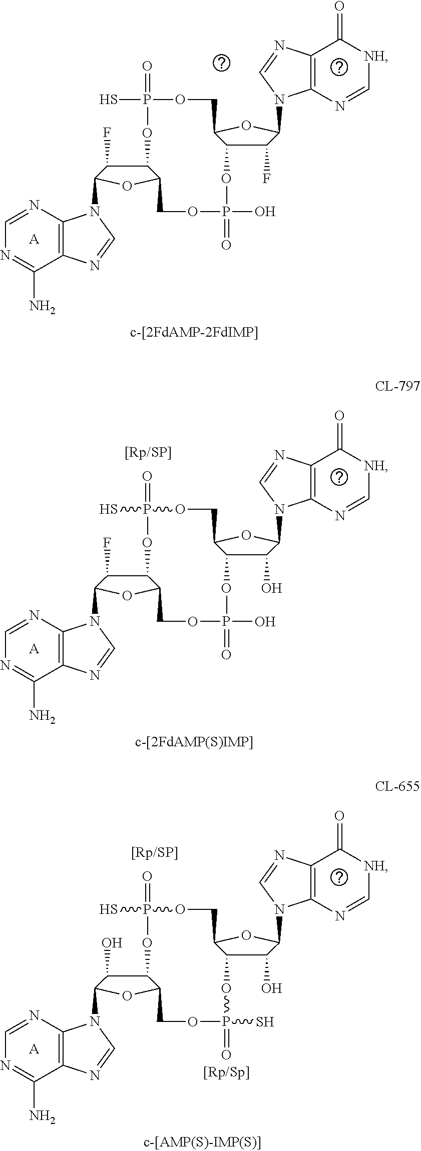

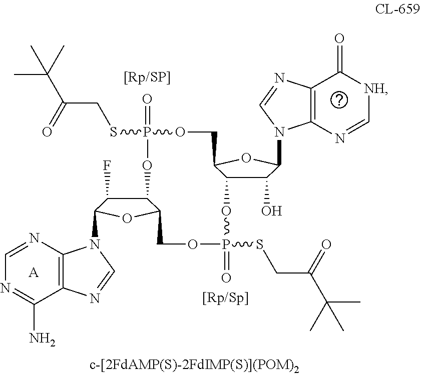

and a pharmaceutically acceptable salt thereof. See WO 2016/096174A1.

In other aspects, the STING agonist useful for the present disclosure comprises a compound having the following formula:

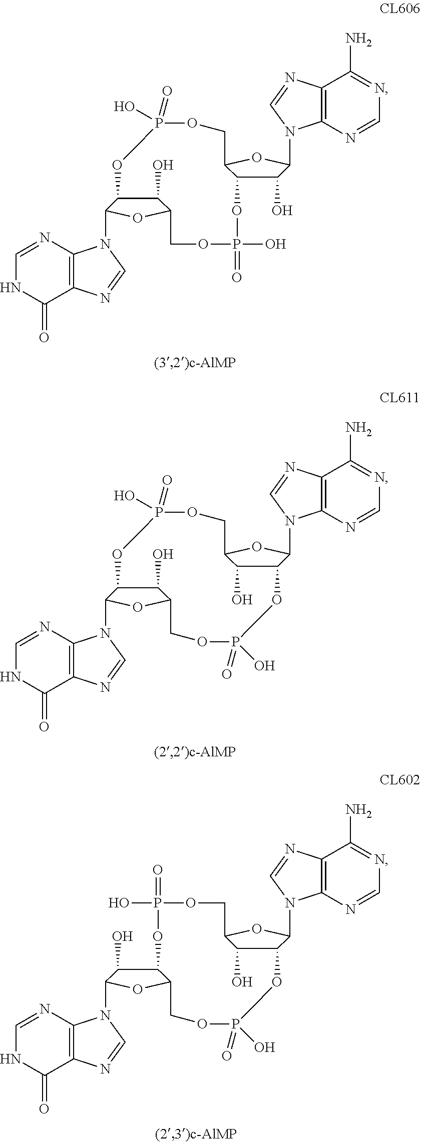 [SMILES: Nc1ncnc2c1ncn2C1OC2COP(=O)(O)O[C@H]3C(n4cnc5c(=O)[nH]cnc54)OC(COP(=O)(O)O[C@H]2[C@H]1O)[C@H]3O ; Nc1ncnc2c1ncn2C1OC2COP(=O)(O)O[C@H]3C(n4cnc5c(=O)[nH]cnc54)OC(COP(=O)(O)O[C@@H]1[C@@H]2O)[C@H]3O ; Nc1ncnc2c1ncn2C1OC2COP(=O)(O)O[C@@H]3C(COP(=O)(O)O[C@@H]1[C@@H]2O)OC(n1cnc2c(=O)[nH]cnc21)[C@@H]3O]

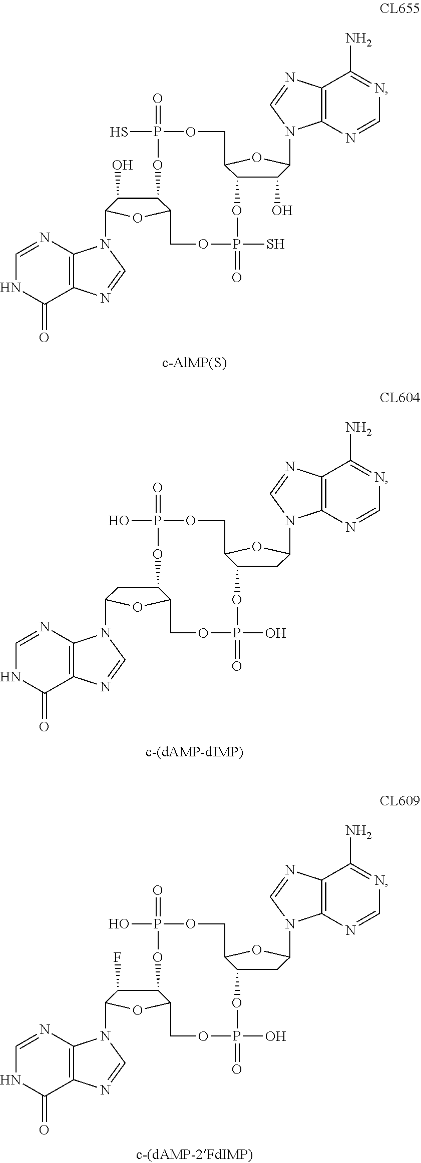 [SMILES: Nc1ncnc2c1ncn2C1OC2COP(=O)(S)O[C@@H]3C(COP(=O)(S)O[C@H]2[C@H]1O)OC(n1cnc2c(=O)[nH]cnc21)[C@@H]3O ; Nc1ncnc2c1ncn2C1C[C@@H]2OP(=O)(O)OCC3OC(n4cnc5c(=O)[nH]cnc54)C[C@@H]3OP(=O)(O)OCC2O1 ; Nc1ncnc2c1ncn2C1C[C@@H]2OP(=O)(O)OCC3OC(n4cnc5c(=O)[nH]cnc54)[C@H](F)[C@@H]3OP(=O)(O)OCC2O1]

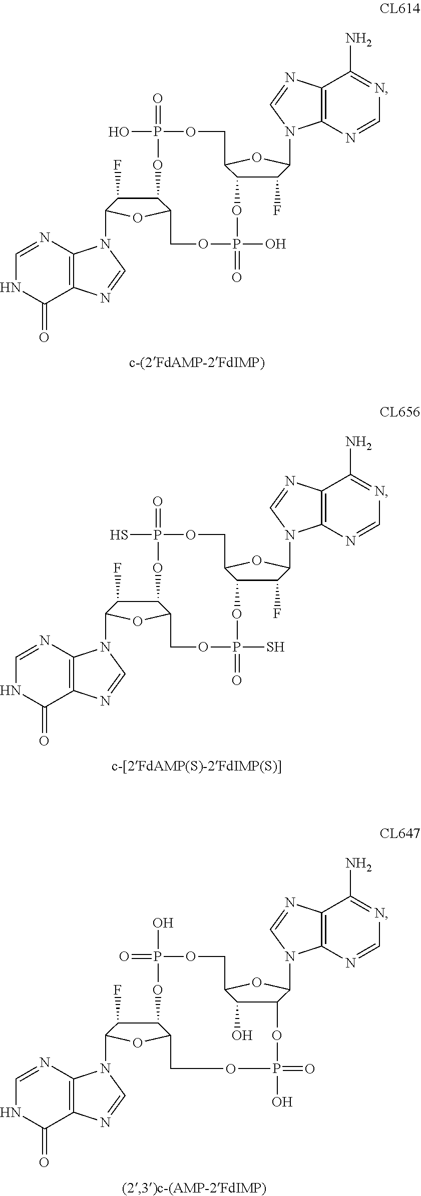 [SMILES: Nc1ncnc2c1ncn2C1OC2COP(=O)(O)O[C@@H]3C(COP(=O)(O)O[C@H]2[C@H]1F)OC(n1cnc2c(=O)[nH]cnc21)[C@@H]3F ; Nc1ncnc2c1ncn2C1OC2COP(=O)(S)O[C@@H]3C(COP(=O)(S)O[C@H]2[C@H]1F)OC(n1cnc2c(=O)[nH]cnc21)[C@@H]3F ; Nc1ncnc2c1ncn2C1OC2COP(=O)(O)O[C@@H]3C(COP(=O)(O)OC1[C@@H]2O)OC(n1cnc2c(=O)[nH]cnc21)[C@@H]3F]

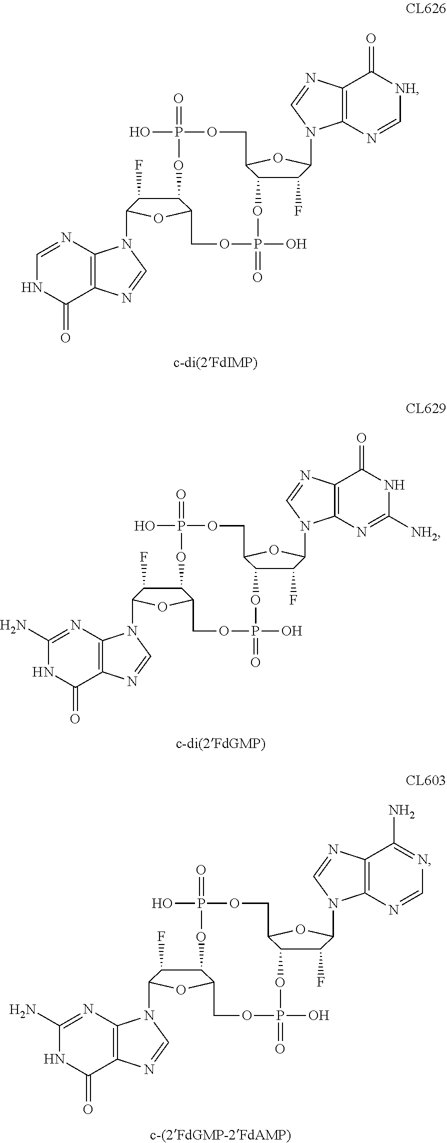 [SMILES: O=c1[nH]cnc2c1ncn2C1OC2COP(=O)(O)O[C@@H]3C(COP(=O)(O)O[C@H]2[C@H]1F)OC(n1cnc2c(=O)[nH]cnc21)[C@@H]3F ; Nc1nc2c(ncn2C2OC3COP(=O)(O)O[C@@H]4C(COP(=O)(O)O[C@H]3[C@H]2F)OC(n2cnc3c(=O)[nH]c(N)nc32)[C@@H]4F)c(=O)[nH]1 ; Nc1nc2c(ncn2C2OC3COP(=O)(O)O[C@@H]4C(COP(=O)(O)O[C@H]3[C@H]2F)OC(n2cnc3c(N)ncnc32)[C@@H]4F)c(=O)[nH]1]

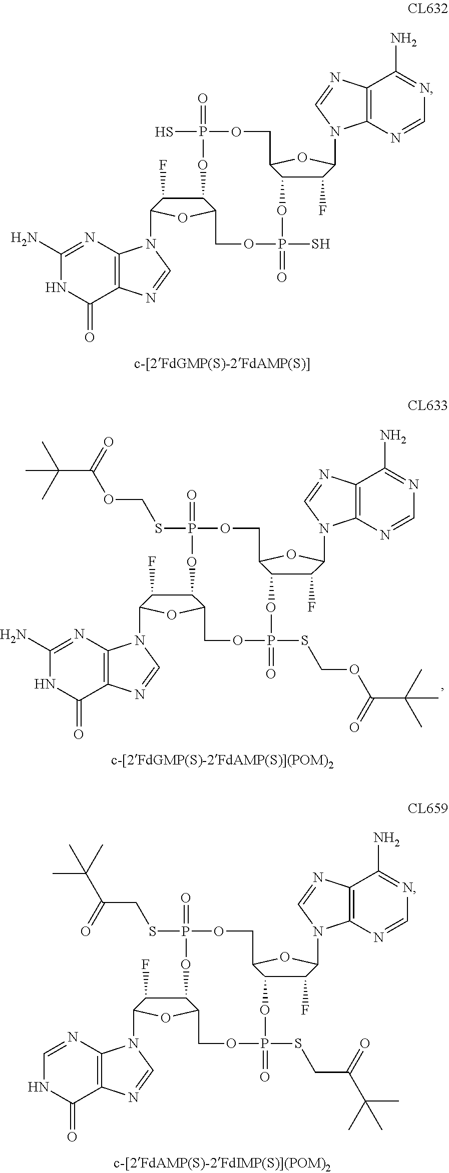 [SMILES: Nc1nc2c(ncn2C2OC3COP(=O)(S)O[C@@H]4C(COP(=O)(S)O[C@H]3[C@H]2F)OC(n2cnc3c(N)ncnc32)[C@@H]4F)c(=O)[nH]1 ; CC(C)(C)C(=O)OCSP1(=O)OCC2OC(n3cnc4c(=O)[nH]c(N)nc43)[C@H](F)[C@@H]2OP(=O)(SCOC(=O)C(C)(C)C)OCC2OC(n3cnc4c(N)ncnc43)[C@H](F)[C@@H]2O1 ; CC(C)(C)C(=O)CSP1(=O)OCC2OC(n3cnc4c(=O)[nH]cnc43)[C@H](F)[C@@H]2OP(=O)(SCC(=O)C(C)(C)C)OCC2OC(n3cnc4c(N)ncnc43)[C@H](F)[C@@H]2O1]

or any pharmaceutically acceptable salts thereof.

In some aspects, the STING agonist useful for the present disclosure comprises a compound having the following formula:

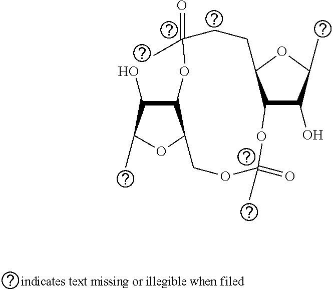

wherein each symbol is defined in WO 2014/093936, the content of which is incorporated herein by reference in its entirety.

In some aspects, the STING agonist useful for the present disclosure comprises a compound having the following formula:

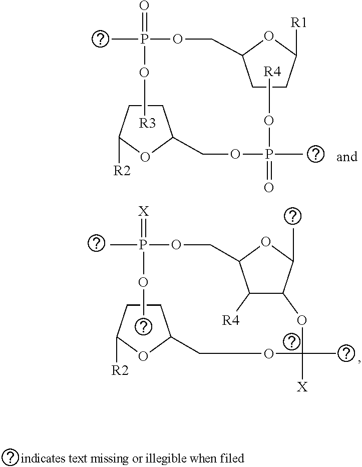

wherein each symbol is defined in WO 2014/189805, the content of which is incorporated herein by reference in its entirety.

In some aspects, the STING agonist useful for the present disclosure comprises a compound having the following formula:

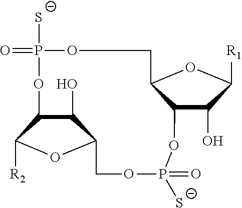

wherein each symbol is defined in WO 2015/077354, the content of which is incorporated herein by reference in its entirety. See also Cell reports 11, 1018-1030 (2015).

In some aspects, the STING agonist useful for the present disclosure comprises c-di-AMP, c-di-GMP, c-di-IMP, c-AMP-GMP, c-AMP-IMP, and c-GMP-IMP, described in WO 2013/185052 and Sci. Transl. Med. 283,283ra52 (2015), which are incorporated herein by reference in their entireties.

In some aspects, the STING agonist useful for the present disclosure comprises a compound having the following formula:

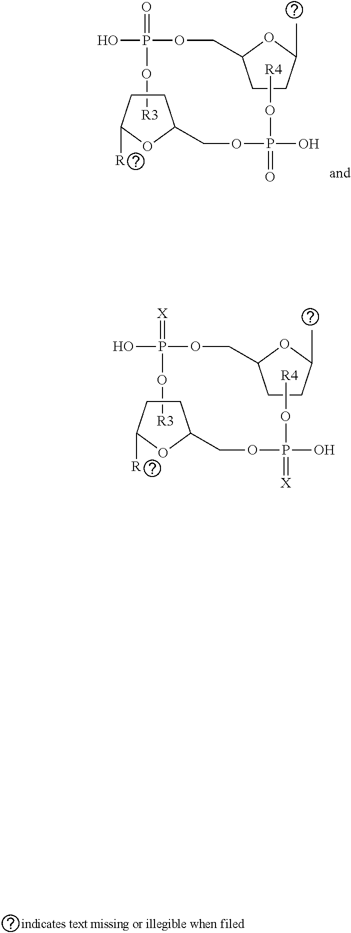

wherein each symbol is defined in WO 2014/189806, the content of which is incorporated herein by reference in its entirety.

In some aspects, the STING agonist useful for the present disclosure comprises a compound having the following formula:

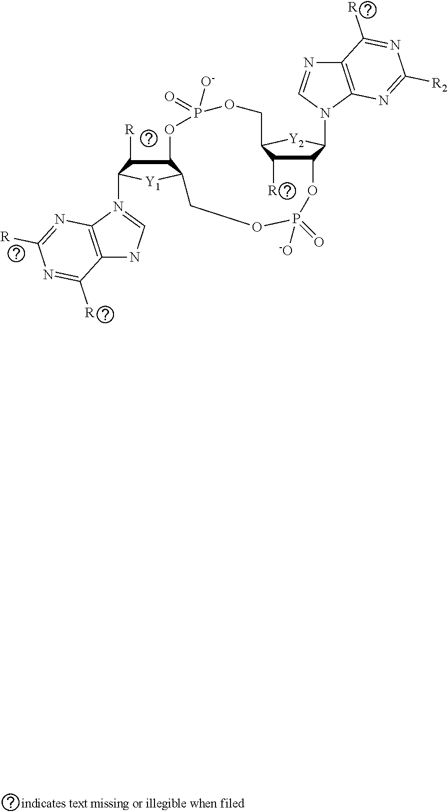

wherein each symbol is defined in WO 2015/185565, the content of which is incorporated herein by reference in its entirety.

In some aspects, the STING agonist useful for the present disclosure comprises a compound having the following formula:

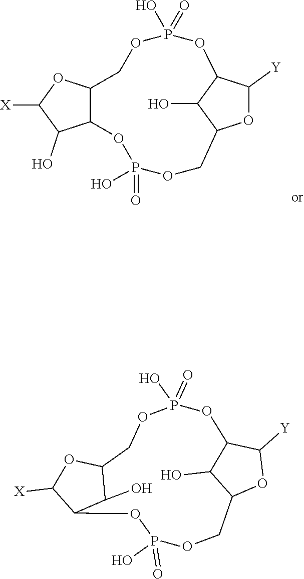

wherein each symbol is defined in WO 2014/179760, the content of which is incorporated herein by reference in its entirety.

In some aspects, the STING agonist useful for the present disclosure comprises a compound having the following formula:

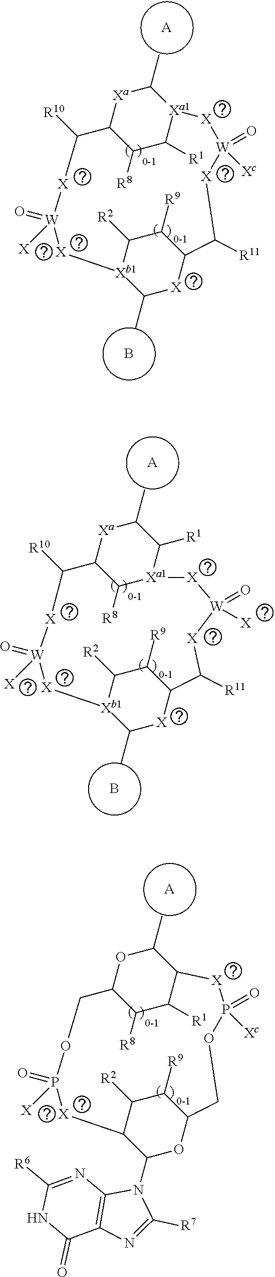

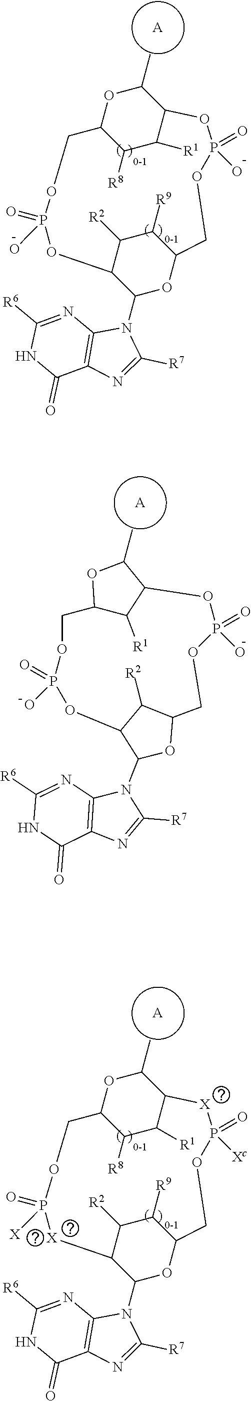

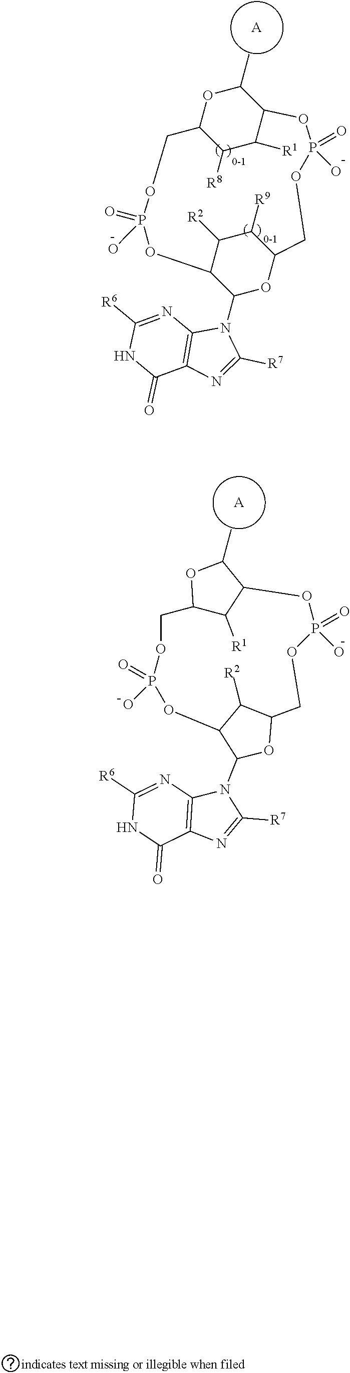

wherein each symbol is defined in WO 2014/179335, the content of which is incorporated herein by reference in its entirety.

In some aspects, the STING agonist useful for the present disclosure comprises a compound having the following formula:

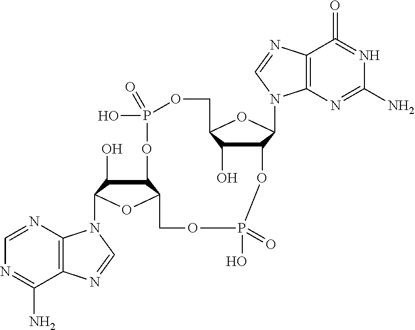

described in WO 2015/017652, the content of which is incorporated herein by reference in its entirety.

In some aspects, the STING agonist useful for the present disclosure comprises a compound having the following formula:

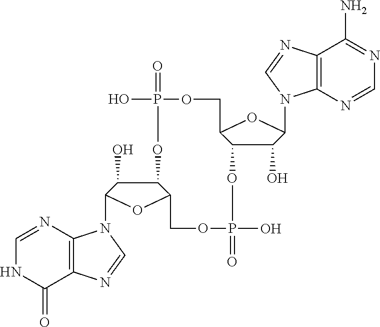 [SMILES: Nc1ncnc2c1ncn2C1OC2COP(=O)(O)O[C@@H]3C(COP(=O)(O)O[C@H]2[C@H]1O)OC(n1cnc2c(=O)[nH]cnc21)[C@@H]3O]

described in WO 2016/096577, the content of which is incorporated herein by reference in its entirety.

In some aspects, the STING agonist useful for the present disclosure comprises a compound having the following formula:

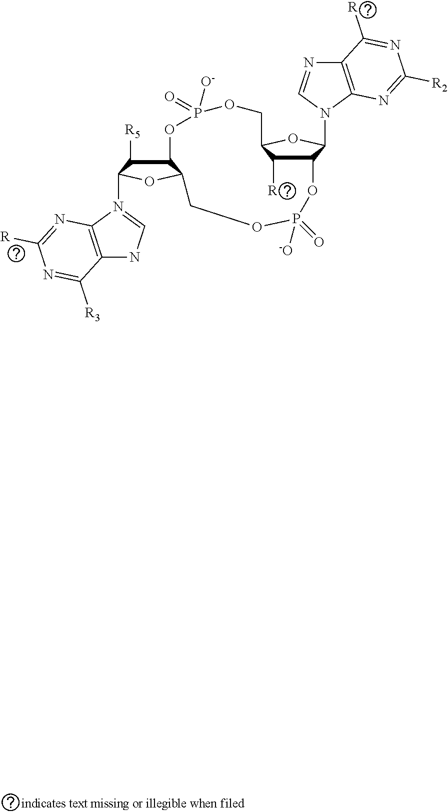

wherein each symbol is defined in WO 2016/120305, the content of which is incorporated herein by reference in its entirety.

In some aspects, the STING agonist useful for the present disclosure comprises a compound having the following formula:

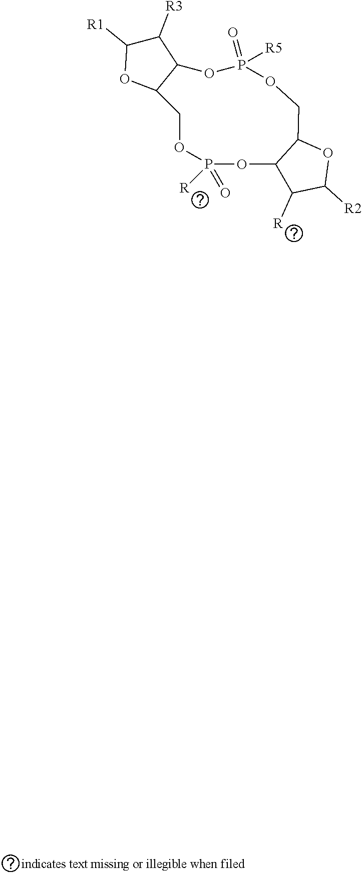

wherein each symbol is defined in WO 2016/145102, the content of which is incorporated herein by reference in its entirety.

In some aspects, the STING agonist useful for the present disclosure comprises a compound having the following formula:

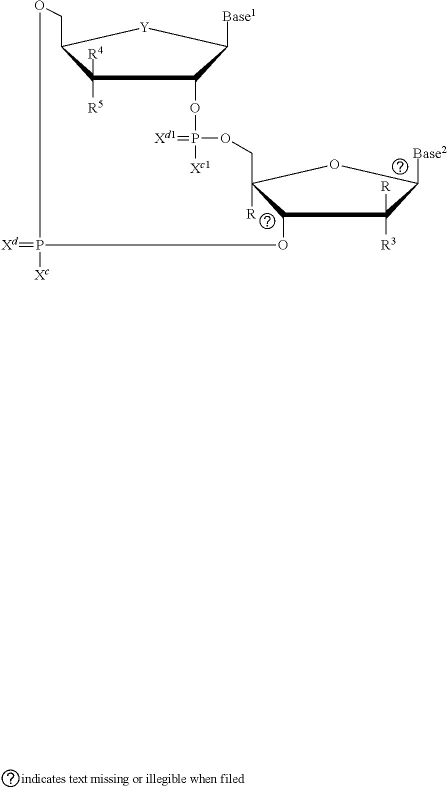

wherein each symbol is defined in WO 2017/027646, the content of which is incorporated herein by reference in its entirety.

In some aspects, the STING agonist useful for the present disclosure comprises a compound having the following formula:

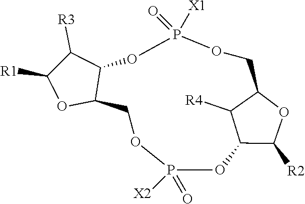

wherein each symbol is defined in WO 2017/075477, the content of which is incorporated herein by reference in its entirety.

In some aspects, the STING agonist useful for the present disclosure comprises a compound having the following formula:

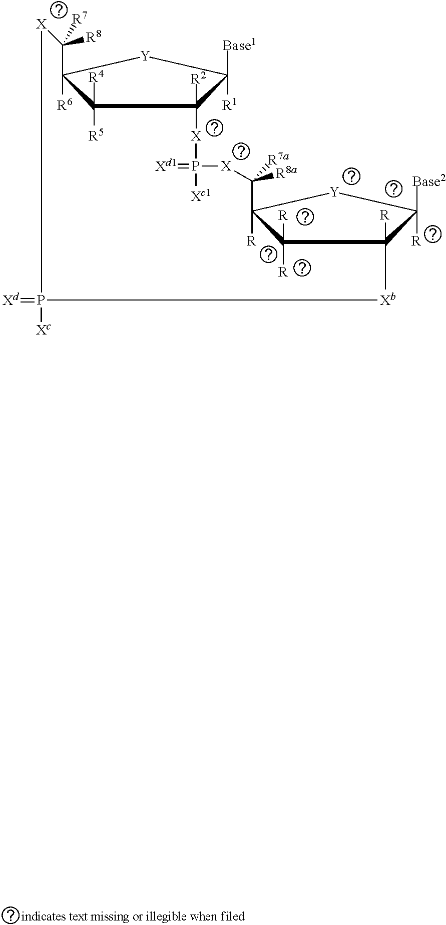

wherein each symbol is defined in WO 2017/027645, the content of which is incorporated herein by reference in its entirety.

In some aspects, the STING agonist useful for the present disclosure comprises a compound having the following formula:

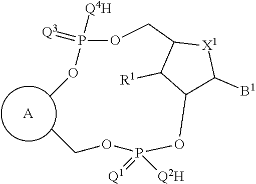

wherein each symbol is defined in WO 2018/100558, the content of which is incorporated herein by reference in its entirety.

In some aspects, the STING agonist useful for the present disclosure comprises a compound having the following formula:

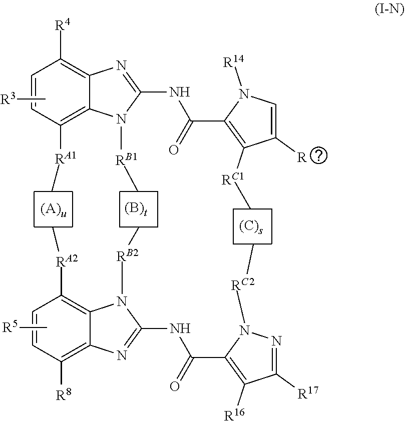

wherein each symbol is defined in WO 2017/175147, the content of which is incorporated herein by reference in its entirety.

In some aspects, the STING agonist useful for the present disclosure comprises a compound having the following formula:

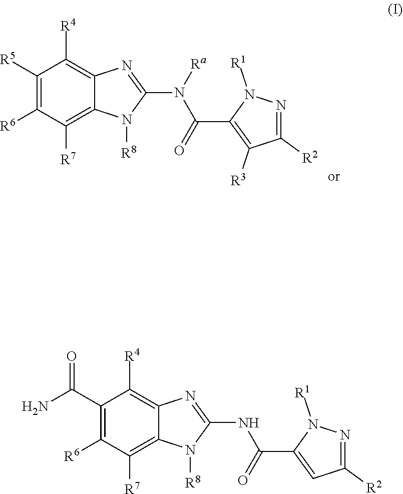

wherein each symbol is defined in WO 2017/175156, the content of which is incorporated herein by reference in its entirety.

In some aspects, the STING agonist useful for the present disclosure is CL606, CL611, CL602, CL655, CL604, CL609, CL614, CL656, CL647, CL626, CL629, CL603, CL632, CL633, CL659, or a pharmaceutically acceptable salt thereof. In some aspects, the STING agonist useful for the present disclosure is CL606 or a pharmaceutically acceptable salt thereof. In some aspects, the STING agonist useful for the present disclosure is CL611 or a pharmaceutically acceptable salt thereof. In some aspects, the STING agonist useful for the present disclosure is CL602 or a pharmaceutically acceptable salt thereof. In some aspects, the STING agonist useful for the present disclosure is CL655 or a pharmaceutically acceptable salt thereof. In some aspects, the STING agonist useful for the present disclosure is CL604 or a pharmaceutically acceptable salt thereof. In some aspects, the STING agonist useful for the present disclosure is CL609 or a pharmaceutically acceptable salt thereof. In some aspects, the STING agonist useful for the present disclosure is CL614 or a pharmaceutically acceptable salt thereof. In some aspects, the STING agonist useful for the present disclosure is CL656 or a pharmaceutically acceptable salt thereof. In some aspects, the STING agonist useful for the present disclosure is CL647 or a pharmaceutically acceptable salt thereof. In some aspects, the STING agonist useful for the present disclosure is CL626 or a pharmaceutically acceptable salt thereof. In some aspects, the STING agonist useful for the present disclosure is CL629 or a pharmaceutically acceptable salt thereof. In some aspects, the STING agonist useful for the present disclosure is CL603 or a pharmaceutically acceptable salt thereof. In some aspects, the STING agonist useful for the present disclosure is CL632 or a pharmaceutically acceptable salt thereof. In some aspects, the STING agonist useful for the present disclosure is CL633 or a pharmaceutically acceptable salt thereof. In some aspects, the STING agonist useful for the present disclosure is CL659 or a pharmaceutically acceptable salt thereof.

In some aspects, the EV, e.g., exosome, comprises a cyclic dinucleotide STING agonist and/or a non-cyclic dinucleotide STING agonist. In some aspects, when several cyclic dinucleotide STING agonists are present on an EV, e.g., exosome, disclosed herein, such STING agonists can be the same or they can be different. In some aspects, when several non-cyclic dinucleotide STING agonists are present, such STING agonists can be the same or they can be different. In some aspects, an EV, e.g., exosome, composition of the present disclosure can comprise two or more populations of EVs, e.g., exosomes, wherein each population of EVs, e.g., exosomes, comprises a different STING agonist or combination thereof.

The STING agonists can also be modified to increase encapsulation of the agonist in an extracellular vesicle or EV (e.g., either unbound in the lumen). In some aspects, the STING agonists are linked to a scaffold moiety, e.g., Scaffold Y. In certain aspects, the modification allows better expression of the STING agonist on the exterior surface of the EV, e.g., exosome, (e.g., linked to a scaffold moiety disclosed herein, e.g., Scaffold X). This modification can include the addition of a lipid binding tag by treating the agonist with a chemical or enzyme, or by physically or chemically altering the polarity or charge of the STING agonist. The STING agonist may be modified by a single treatment, or by a combination of treatments, e.g., adding a lipid binding tag only, or adding a lipid binding tag and altering the polarity. The previous example is meant to be a non-limiting illustrative instance. It is contemplated that any combination of modifications may be practiced.

### III.B. Interleukin-12 (IL-12)

Certain aspects of the present disclosure are directed to methods of administering an extracellular vesicle comprising an IL-12 moiety to a subject in need thereof. In some aspects, the method further comprises administering an EV comprising a STING agonist. In some aspects, the method does not comprise administering an EV comprising a STING agonist. Interleukin 12 (IL-12) is heterodimeric cytokine produced by dendritic cells, macrophages and neutrophils. See, e.g., Interleukin-12 Signaling, Reactome, available at reactome.org/content/detail/R-HSA-9020591; and UniProtKB-P29459 (IL-12A Subunit) P29460 (IL-12B Subunit). It is encoded by the genes Interleukin-12 subunit alpha (IL12A) and Interleukin-12 subunit beta (IL12B), which encode a 35-kDa light chain (p35) and a 40-kDa heavy chain (p40), respectively. The active IL-12 heterodimer is sometimes referred to as p70. The p35 component has homology to single-chain cytokines, while p40 is homologous to the extracellular domains of members of the haematopoietic cytokine-receptor family. The IL-12 heterodimer therefore resembles a cytokine linked to a soluble receptor. IL-12 is involved in the differentiation of naive T cells into Th1 cells and sometimes known as T cell-stimulating factor. IL-12 enhances the cytotoxic activity of Natural Killer cells and CD8+ cytotoxic T lymphocytes. IL-12 also has anti-angiogenic activity, mediated by increased production of CXCL10 via interferon gamma. Non-limiting examples IL-12 moieties usable in the present disclosure can be found, for example, in U.S. Pat. No. 10,723,782, International Publication No. WO 2019/133934 A2, and International Application No. PCT/US2020/028778, each of which is incorporated by reference herein in its entirety.

The IL-12 receptor is a heterodimer formed by Interleukin-12 receptor subunit beta-1 (IL12RB1) and Interleukin-12 receptor subunit beta-2 (IL12RB2), both of which have extensive homology to IL6ST (gp130), the signal transducing receptor subunit of the IL6-like cytokine superfamily. IL-12RB2 is considered to play the key role in IL-12 function, in part because its expression on activated T cells is stimulated by cytokines that promote Th1 cell development and inhibited by those that promote Th2 cells development. In addition, IL-12 binding leads to IL12RB2 tyrosine phosphorylation, which provides binding sites for the kinases Non-receptor tyrosine-protein kinase TYK2 and Tyrosine-protein kinase JAK2. These activate transcription factor proteins in the Signal transducer and activator of transcription (STAT) family, particularly STAT4. is a cytokine that is produced by myeloid and other cell types.

The amino acid sequences for the IL-12 A and B subunits are shown in Table 1A.

TABLE 1A
Human IL-12A and IL-12B subunits amino acid sequences.

| Human IL-12A Subunit (UniProtKB-P29459) | Human IL-12B Subunit (UniProtKB-P29460) |
|---|---|
| (signal peptide) | (signal peptide) |
| MCPARSLLLVATLVLLDHLSLARNLPVATPDPGMFPCLH | MCHQQLVISWFSLVFLASPLVAIWELKKDVYVVELDWY |
| HSQNLLRAVSNMLQKARQTLEFYPCTSEEIDHEDITKDK | PDAPGEMVVLTCDTPEEDGITWTLDQSSEVLGSGKTLTI |
| TSTVEACLPLELTKNESCLNSRETSFITNGSCLASRKTSFM | QVKEFGDAGQYTCHKGGEVLSHSLLLLHKKEDGIWSTD |
| MALCLSSIYEDLKMYQVEFKTMNAKLLMDPKRQIFLDQ | ILKDQKEPKNKTFLRCEAKNYSGRFTCWWLTTISTDLTFS |
| NMLAVIDELMQALNFNSETVPQKSSLEEPDFYKTKIKLCI | VKSSRGSSDPQGVTCGAATLSAERVRGDNKEYEYSVEC |
| LLHAFRIRAVTIDRVMSYLNAS | QEDSACPAAEESLPIEVMVDAVHKLKYENYTSSFFIRDII |
| SEQ ID NO: 11 | KPDPPKNLQLKPLKNSRQVEVSWEYPDTWSTPHSYFSL |
| | TFCVQVQGKSKREKKDRVFTDKTSATVICRKNASISVRA |
| | QDRYYSSSWSEWASVPCS |
| | SEQ ID NO: 12 |

In some aspects, the IL-12 moiety comprises an IL-12 protein. In some aspects, the IL-12 protein comprises full length human IL-12, e.g., IL-12 heterodimer. In some aspects, the IL-12 heterodimer comprises a fusion protein, wherein the IL-12 alpha subunit is covalently linked to the IL-12 beta subunit (SEQ ID NO: 13; Table 1B). In some aspects, the IL-12 moiety comprises the IL-12 alpha subunit. In some aspects, the IL-12 moiety comprises the IL-12 beta subunit.

TABLE 1B
Human IL-12A-IL-12B fusion amino acid sequence.

| | |
|---|---|
| IL-12 | MCHQQLVISWFSLVFLASPLVAIWELKKDVYVVELDWYPDAPGEMVVLTCDTPEED |
| Fusion | GITWTLDQSSEVLGSGKTLTIQVKEFGDAGQYTCHKGGEVLSHSLLLLHKKEDGIW |
| (signal | STDILKDQKEPKNKTFLRCEAKNYSGRFTCWWLTTISTDLTFSVKSSRGSSDPQGV |
| peptide- | TCGAATLSAERVRGDNKEYEYSVECQEDSACPAAEESLPIEVMVDAVHKLKYENYT |
| p40- | SSFFIRDIIKPDPPKNLQLKPLKNSRQVEVSWEYPDTWSTPHSYFSLTFCVQVQGK |
| linker- | SKREKKDRVFTDKTSATVICRKNASISVRAQDRYYSSSWSEWASVPCSGGSGGGSG |
| p35) | GGGSGGGGSGGGSGGRNLPVATPDPGMFPCLHHSQNLLRAVSNMLQKARQTLEFYP |
| SEQ ID | CTSEEIDHEDITKDKTSTVEACLPLELTKNESCLNSRETSFITNGSCLASRKTSFM |
| NO: 13 | MALCLSSIYEDLKMYQVEFKTMNAKLLMDPKRQIFLDQNMLAVIDELMQALNFNSE |
| | TVPQKSSLEEPDFYKTKIKLCILLHAFRIRAVTIDRVMSYLNAS |

In some aspects, the IL-12 heterodimer comprises an IL-12 alpha subunit covalently linked to an IL-12 beta subunit by a linker. In some aspects, the linker comprises one or more amino acids. In some aspects, the linker is a linker disclosed herein. In some aspect, the linker comprises a Gly/Ser linker. In some aspects, the linker is a cleavable linker. In some aspects, the linker comprises disulfide bond.

In some aspects, the IL-12 moiety comprises a molecule having IL-12 activity. In some aspects, the molecule having IL-12 activity is an IL-12 analog. In some aspects, the molecule having IL-12 activity comprises a molecule which activates IL-12 receptor.

In some aspects, the IL-12 moiety comprises a nucleic acid molecule encoding an IL-12 protein, e.g., an IL-12 alpha subunit, an IL-12 beta subunit, and/or an IL-12 heterodimer. In some aspects, the nucleic acid molecule encodes an IL-12 alpha subunit. In some aspects, the nucleic acid molecule encodes an IL-12 beta subunit. In some aspects, the nucleic acid molecule encodes an IL-12 alpha subunit and an IL-12 beta subunit. In some aspects, the nucleic acid molecule encodes an IL-12 alpha subunit covalently linked to an IL-12 beta subunit. In some aspects, the nucleic acid molecule encodes an IL-12 heterodimer.

In some aspects, the IL-12 moiety comprises a nucleic acid molecule, wherein the nucleic acid molecule is packed in a vector. In some aspects, the vector is a viral vector. In some aspects, the vector is based on a DNA virus, such as adenovirus, adeno-associated virus (AAV) and herpes virus, as well as retroviral based vectors. In some aspects, the vector is a lentivirus. In some aspects, the virus is an AAV.

### III.C. Scaffold-Engineered EVs, e.g., Exosomes

In some aspects, EVs of the present disclosure comprise a membrane modified in its composition. For example, their membrane compositions can be modified by changing the protein, lipid, or glycan content of the membrane.

In some aspects, the surface-engineered EVs are generated by chemical and/or physical methods, such as PEG-induced fusion and/or ultrasonic fusion. In other aspects, the surface-engineered EVs, e.g., exosomes, are generated by genetic engineering. EVs produced from a genetically-modified producer cell or a progeny of the genetically-modified cell can contain modified membrane compositions. In some aspects, surface-engineered EVs, e.g., exosomes, have scaffold moiety (e.g., exosome protein, e.g., Scaffold X) at a higher or lower density (e.g., higher number) or include a variant or a fragment of the scaffold moiety.

For example, surface-engineered EVs (e.g., Scaffold X-engineered or Scaffold Y-engineered EVs) can be produced from a cell (e.g., HEK293 cells) transformed with an exogenous sequence encoding a scaffold moiety (e.g., exosome proteins, e.g., Scaffold X and/or Scaffold Y) or a variant or a fragment thereof. EVs including scaffold moiety expressed from the exogenous sequence can include modified membrane compositions.

Various modifications or fragments of the scaffold moiety can be used for the aspects of the present disclosure. For example, scaffold moiety modified to have enhanced affinity to a binding agent can be used for generating surface-engineered EVs that can be purified using the binding agent. Scaffold moieties modified to be more effectively targeted to EVs, e.g., exosomes, and/or membranes can be used. Scaffold moieties modified to comprise a minimal fragment required for specific and effective targeting to EVs, e.g., exosomes, membranes can be also used.

In some aspects, a STING agonist disclosed herein is expressed on the surface of an EV, e.g., exosome, as a fusion protein, e.g., fusion protein of a STING agonist to a Scaffold X and/or Scaffold Y. For example, the fusion protein can comprise a STING agonist disclosed herein linked to a scaffold moiety (e.g., Scaffold X or Scaffold Y).

### III.C.1. Scaffold X Proteins

In certain aspects, Scaffold X comprises the PTGFRN protein, BSG protein, IGSF2 protein, IGSF3 protein, IGSF8 protein, ITGB1 protein, ITGA4 protein, SLC3A2 protein, ATP transporter protein, Lamp-1 protein, Lamp-2 protein, CD13 protein, CD86 protein, Flotillin protein, Syntaxin-3 protein, CD2 protein, CD36 protein, CD40 protein, CD40L protein, CD41a protein, CD44 protein, CD45 protein, ICAM-1 protein, Integrin alpha4 protein, L1CAM protein, LFA-1 protein, Mac-1 alpha and beta protein, Vti-1A and B protein, CD3 epsilon and zeta protein, CD9 protein, CD18 protein, CD37 protein, CD53 protein, CD63 protein, CD81 protein, CD82 protein, CXCR4 protein, FcR protein, GluR2/3 protein, HLA-DM (MHC II) protein, immunoglobulins protein, MHC-I or MHC-II components protein, TCR beta protein, tetraspanin protein, or a fragment or a variant thereof.

In some aspects, the surface-engineered EVs, e.g., exosomes (e.g., Scaffold X-engineered EVs, e.g., exosomes) described herein demonstrate superior characteristics compared to EVs, e.g., exosomes, known in the art. For example, surface (e.g., Scaffold X)-engineered contain modified proteins more highly enriched on their surface than naturally occurring EVs, e.g., exosomes, or the EVs, e.g., exosomes, produced using conventional exosome proteins. Moreover, the surface-engineered EVs, e.g., exosomes, (e.g., Scaffold X-engineered EVs, e.g., exosomes) of the present invention can have greater, more specific, or more controlled biological activity compared to naturally occurring EVs, e.g., exosomes, or the EVs, e.g., exosomes, produced using conventional exosome proteins.

In other aspects, the EVs, e.g., exosomes, of the present disclosure contains a STING agonist and a Scaffold X, wherein the STING agonist is linked to the Scaffold X. In some aspects, the EVs, e.g., exosomes, of the present disclosure comprises a STING agonist and a Scaffold X, wherein the STING agonist is not linked to the Scaffold X.

In some aspects, Scaffold X useful for the present disclosure comprises Prostaglandin F2 receptor negative regulator (the PTGFRN polypeptide). The PTGFRN protein can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full length amino acid sequence of the human PTGFRN protein (Uniprot Accession No. Q9P2B2) is shown at Table 2 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least five, at least 10, at least 15, at least 20, at least 25, at least 30, at least 40, at least 50, at least 70, at least 80, at least 90, at least 100, at least 110, at least 120, at least 130, at least 140, at least 150 amino acids at the N terminus of the transmembrane domain, (ii) at least five, at least 10, at least 15, at least 20, or at least 25 amino acids at the C terminus of the transmembrane domain, or both (i) and (ii).

In some aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 33.

TABLE 2A
Exemplary Scaffold X Protein Sequences

| Protein | Sequence |
|---|---|
| The PTGFRN | MGRLASRPLLLALLSLALCRGRVVRVPTATLVRVVGTELVIPCNVSDYDGPSEQNFDWSFSSLGSS |
| Protein | FVELASTWEVGFPAQLYQERLQRGEILLRRTANDAVELHIKNVQPSDQGHYKCSTPSTDATVQGNY |
| (SEQ ID NO: | EDTVQVKVLADSLHVGPSARPPPSLSLREGEPFELRCTAASASPLHTHLALLWEVHRGPARRSVLA |
| 1) | LTHEGRFHPGLGYEQRYHSGDVRLDTVGSDAYRLSVSRALSADQGSYRCIVSEWIAEQGNWQEIQE |
| | KAVEVATVVIQPSVLRAAVPKNVSVAEGKELDLTCNITTDRADDVRPEVTWSFSRMPDSTLPGSRV |
| | LARLDRDSLVHSSPHVALSHVDARSYHLLVRDVSKENSGYYYCHVSLWAPGHNRSWHKVAEAVSSP |
| | AGVGVTWLEPDYQVYLNASKVPGFADDPTELACRVVDTKSGEANVRFTVSWYYRMNRRSDNVVTSE |
| | LLAVMDGDWTLKYGERSKQRAQDGDFIFSKEHTDTFNFRIQRTTEEDRGNYYCVVSAWTKQRNNSW |
| | VKSKDVFSKPVNIFWALEDSVLVVKARQPKPFFAAGNTFEMTCKVSSKNIKSPRYSVLIMAEKPVG |
| | DLSSPNETKYIISLDQDSVVKLENWTDASRVDGVVLEKVQEDEFRYRMYQTQVSDAGLYRCMVTAW |
| | SPVRGSLWREAATSLSNPIEIDFQTSGPIFNASVHSDTPSVIRGDLIKLFCIITVEGAALDPDDMA |
| | FDVSWFAVHSFGLDKAPVLLSSLDRKGIVTTSRRDWKSDLSLERVSVLEFLLQVHGSEDQDFGNYY |
| | CSVTPWVKSPTGSWQKEAEIHSKPVFITVKMDVLNAFKYPLLIGVGLSTVIGLLSCLIGYCSSHWC |
| | CKKEVQETRRERRRLMSMEMD |
| The PTGFRN | GPIFNASVHSDTPSVIRGDLIKLFCIITVEGAALDPDDMAFDVSWFAVHSFGLDKAPVLLSSLDRK |
| protein | GIVTTSRRDWKSDLSLERVSVLEFLLQVHGSEDQDFGNYYCSVTPWVKSPTGSWQKEAEIHSKPVF |
| Fragment | ITVKMDVLNAFKYPLLIGVGLSTVIGLLSCLIGYCSSHWCCKKEVQETRRERRRLMSMEM |
| (SEQ ID NO: | 687-878 of SEQ ID NO: 1 |
| 33) | |

In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 2, 3, 4, 5, 6, 7, or 8.

Non-limiting examples of other Scaffold X proteins that can be used to link a STING agonist to the surface of EVs, e.g., exosomes, can be found at U.S. Pat. Nos. 10,195,290 B1 and 10,561,740 B2, each of which is incorporated by reference in its entirety.

In some aspects, Scaffold X described herein can also be used to link a STING agonist and/or an IL-12 moiety on the luminal surface and/or on the exterior surface of the EVs, e.g., exosomes, at the same time. For example, the PTGFRN polypeptide can be used to link a STING agonist and/or an IL-12 moiety inside the lumen in addition to the surface of the EV, e.g., exosome. In some aspects, a Scaffold X can be used to link a STING agonist and an additional therapeutic agent (e.g., an IL-12 moiety) to the EVs, e.g., exosomes, (e.g., payload). Therefore, in certain aspects, Scaffold X disclosed herein can be used for dual purposes.

In some aspects, EVs, e.g., exosomes, of the present disclosure comprise an internal space (i.e., lumen) that is different from that of the naturally occurring EVs, e.g., exosomes. For example, the EV, e.g., exosome, can be changed such that the composition in the luminal side of the EV, e.g., exosome, has the protein, lipid, or glycan content different from that of the naturally-occurring EVs, e.g., exosomes.

In some aspects, engineered EVs, e.g., exosomes, can be produced from a cell transformed with an exogenous sequence encoding a scaffold moiety (e.g., exosome proteins, e.g., Scaffold Y) or a modification or a fragment of the scaffold moiety that changes the composition or content of the luminal side of the EV, e.g., exosome. Various modifications or fragments of the exosome protein that can be expressed in the luminal side of the EV, e.g., exosome, can be used for the aspects of the present disclosure.

In some aspects, a STING agonist and/or an IL-12 moiety disclosed herein is in the lumen of the EV, e.g., exosome (i.e., encapsulated). In some aspects, a STING agonist and/or an IL-12 moiety is linked to the luminal surface of the EV, e.g., exosome. As used herein, when a molecule (e.g., a STING agonist and/or an IL-12 moiety) is described as “in the lumen” of the EV, e.g., exosome, it means that the molecule is located within the EV, e.g., exosome (e.g., associated), but is not linked to any molecule on the luminal surface of EVs. In other aspects, a STING agonist and/or an IL-12 moiety is expressed on the luminal surface of the EV, e.g., exosome as a fusion molecule, e.g., fusion molecule of a STING agonist to a scaffold moiety (e.g., Scaffold X or Scaffold Y).

In some aspects, the combination therapy of the present disclosure comprises administering a first EV comprising a STING agonist in the lumen, and a second EV comprising an IL-12 on the exterior surface of the EV via a PTGFRN protein.

### III.C.2. Scaffold Y Proteins

In some aspects, a STING agonist and/or an IL-12 moiety is expressed on the luminal surface of the EV, e.g., exosome as a fusion molecule, e.g., fusion molecule of a STING agonist to a Scaffold Y moiety. In some aspects, engineered EVs, e.g., exosomes, can be produced from a cell transformed with an exogenous sequence encoding a scaffold moiety (e.g., exosome proteins, e.g., Scaffold Y) or a modification or a fragment of the scaffold moiety that changes the composition or content of the luminal surface of the EV, e.g., exosome. Various modifications or fragments of the exosome protein that can be expressed on the luminal surface of the EV, e.g., exosome, can be used for the aspects of the present disclosure.

In some aspects, the exosome proteins that can change the luminal surface of the EVs, e.g., exosomes, include, but are not limited to, the myristoylated alanine rich Protein Kinase C substrate (MARCKS) protein, the myristoylated alanine rich Protein Kinase C substrate like 1 (MARCKSL1) protein, the brain acid soluble protein 1 (BASP1) protein, or any combination thereof.

Non-limiting examples of the Scaffold Y protein useful for the present disclosure are disclosed herein. In some aspects, the Scaffold Y protein comprises an amino acid sequence selected from SEQ ID NOs: 411, 438, 446, and 455-567. In some aspects, the Scaffold Y protein consists of an amino acid sequence selected from SEQ ID NOs: 411, 438, 446, and 455-567. In some aspects, the Scaffold Y protein comprises or consists of any Scaffold Y protein disclosed in International Publ. No. WO/2019/099942 or WO 2020/101740, each of which is incorporated herein by reference in its entirety.

In some aspects, a STING agonist is associated with a Scaffold X in an EV, and an IL-12 moiety is associated with the Scaffold X moiety. In some aspects, a STING agonist is associated with a first Scaffold X in an EV, and an IL-12 moiety is associated with a second Scaffold X moiety. In some aspects, a STING agonist is associated with a Scaffold X in an EV, and an IL-12 moiety is associated with a Scaffold Y moiety. In some aspects, a STING agonist is associated with a first Scaffold Y in an EV, and an IL-12 moiety is associated with a second Scaffold Y moiety. In some aspects, a STING agonist is not associated with a scaffold protein in an EV, and an IL-12 moiety is associated with a Scaffold Y moiety. In some aspects, a STING agonist is not associated with a scaffold protein in an EV, and an IL-12 moiety is associated with a Scaffold X moiety. In some aspects, an IL-12 moiety is not associated with a scaffold protein in an EV, and STING agonist is associated with a Scaffold Y moiety. In some aspects, an IL-12 moiety is not associated with a scaffold protein in an EV, and STING agonist is associated with a Scaffold X moiety.

### III.D. Linkers

The EVs of the present disclosure can comprises one or more linkers that link the STING agonist and/or the IL-12 moiety to EVs or to a scaffold moiety, e.g., Scaffold X on the exterior surface of the EVs. In some aspects, the STING agonist and/or the IL-12 moiety is linked to the EVs directly or in a scaffold moiety on the EVs by a linker. In some aspects, the STING agonist and/or the IL-12 is linked to a lipid bilayer of the EV, e.g., by a linker. The linker can be any chemical moiety known in the art.

In some aspects, the term “linker” refers to a peptide or polypeptide sequence (e.g., a synthetic peptide or polypeptide sequence) or to a non-polypeptide. In some aspects, two or more linkers can be linked in tandem. Generally, linkers provide flexibility or prevent/ameliorate steric hindrances. Linkers are not typically cleaved; however in certain aspects, such cleavage can be desirable. Accordingly, in some aspects a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

Linkers may be susceptible to cleavage (“cleavable linker”) thereby facilitating release of the STING Agonist or other payloads. In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof. In some aspects, the linker comprises a non-cleavable liker.

### III.E. Producer Cells and Modifications

EVs, e.g., exosomes, can be produced from a cell grown in vitro or a body fluid of a subject. When EVs, e.g., exosomes, are produced from in vitro cell culture, various producer cells, e.g., HEK293 cells, can be used. Additional cell types that can be used for the production of the lumen-engineered EVs, e.g., exosomes, described herein include, without limitation, mesenchymal stem cells, T-cells, B-cells, dendritic cells, macrophages, and cancer cell lines. Further examples include: Chinese hamster ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, and RPTEC/TERT1 cells. In certain aspects, a producer cell is not a dendritic cell, macrophage, B cell, mast cell, neutrophil, Kupffer-Browicz cell, cell derived from any of these cells, or any combination thereof.

Some aspects may also include genetically modifying the EV, e.g., exosome, to comprise one or more exogenous sequences, e.g., PTGFRN linked to IL-12 to produce modified EVs that express exogenous proteins on the vesicle surface.

More specifically, the EV, e.g., exosome, of the present can be produced from a cell transformed with a sequence encoding one or more additional exogenous proteins including, but not limited to ligands, cytokines, or antibodies, or any combination thereof. These additional exogenous proteins may enable activation or modulation of additional immune stimulatory signals in combination with the STING agonist. Exemplary additional exogenous proteins contemplated for use include the proteins, ligands, and other molecules described in detail in U.S. Patent Application 62/611,140, International Publication No. WO/2019/133934, and U.S. Pat. Nos. 10,195,290 B1 and 10,561,740 B2, each of which is incorporated herein by reference in its entirety. In some aspects, the EV, e.g., exosome, is further modified with a ligand comprising CD40L, OX40L, or CD27L. In some aspects, the EV, e.g., exosome, is further modified with a cytokine comprising IL-7, IL-12, or IL-15. Any of the one or more exosome proteins described herein can be expressed from a plasmid, an exogenous sequence inserted into the genome or other exogenous nucleic acid such as a synthetic messenger RNA (mRNA).

In some aspects, the EV, e.g., exosome, is further modified to display an antagonistic antibody or an agonistic antibody or a fragment thereof on the EV, e.g., exosome, surface to direct EV uptake, activate, or block cellular pathways to enhance the combinatorial effect of the STING agonist. In some specific aspects, the antibody or fragment thereof is an antibody against DEC205, CLEC9A, CLEC6, DCIR, DC-SIGN, LOX-1, or Langerin. The producer cell may be modified to comprise an additional exogenous sequence encoding for an antagonistic antibody or an agonistic antibody. Alternatively, the antagonistic antibody or agonistic antibody may be covalently linked or conjugated to the EV, e.g., exosome, via any appropriate linking chemistry known in the art. Non-limiting examples of appropriate linking chemistry include amine-reactive groups, carboxyl-reactive groups, sulfhydryl-reactive groups, aldehyde-reactive groups, photoreactive groups, ClickIT chemistry, biotin-streptavidin or other avidin conjugation, or any combination thereof.

### IV. Method of Producing EVs with STING Agonists

### IV.A. Methods for Encapsulating STING Agonists in EVs

STING agonists can be encapsulated in EVs, e.g., exosomes, via any appropriate technique known in the art. It is contemplated that all known manners of loading biomolecules into EVs, e.g., exosomes, are deemed suitable for use herein. Such techniques include passive diffusion, electroporation, chemical or polymeric transfection, viral transduction, mechanical membrane disruption or mechanical shear, or any combination thereof. The STING agonist and an EV, e.g., exosome, may be incubated in an appropriate buffer during encapsulation.

In one aspect, a STING agonist is encapsulated by an EV, e.g., exosome, by passive diffusion. The STING agonist and the EV, e.g., exosome, may be mixed together and incubated for a time period sufficient for the STING agonist to diffuse through the vesicle lipid bilayer, thereby becoming encapsulated in the EV, e.g., exosome. The STING agonist and the EV, e.g., exosome, may be incubated together for between about 1 to 30 hours, 2 to 24 hours, 4 to 18 hours, 6 to 16 hours, 8 to 14 hours, 10 to 12 hours, 6 to 12 hours, 12 to 20 hours, 14 to 18 hours, or 20 to 30 hours. The STING agonist and the EV, e.g., exosome, may be incubated together for about 2 hours, 4 hours, 6, hours, 8, hours, 10, hours, 12 hours, 14 hours, 16 hours, 18 hours, 20 hours, 22 hours, 24 hours, 26 hours, or 30 hours.

The buffer conditions of the solution of EVs, e.g., exosomes, may also be altered to optimize encapsulation of the STING agonist. In one aspect, the buffer may be a phosphate buffered saline (PBS) with sucrose. PBS is a well-known buffer to those skilled in the art. Additional buffer modifications may also be used, such as shear protectants, viscosity modifiers, and/or solutes that affect vesicle structural properties. Excipients may also be added to improve the efficiency of the STING agonist encapsulation such as membrane softening materials and molecular crowding agents. Other modifications to the buffer may include specific pH ranges and/or concentrations of salts, organic solvents, small molecules, detergents, zwitterions, amino acids, polymers, and/or any combination of the above including multiple concentrations.

The temperature of the solution of EVs, e.g., exosomes, and STING agonists during incubation may be changed to optimize encapsulation of the STING agonist. The temperature may be room temperature. The temperature may be between about 15° C. to 90° C., 15-30° C., 30-50° C., 50-90° C. The temperature may be about 15° C., 20° C., 35° C., 30° C., 35° C., 37° C., 40° C., 45° C., 50° C., 55° C., 60° C., 65° C., 70° C., 75° C., 80° C., 85° C., or 90° C.

The concentration of STING agonist during the incubation of the agonist with the EVs, e.g., exosomes, may also be altered to optimize encapsulation of the STING agonist. The concentration of agonist may be between at least 0.01 mM and 100 mM STING agonist. The concentration of the agonist may be at least 0.01-1 mM, 1-10 mM, 10-50 mM, or 50-100 mM. The concentration of the agonist may be at least 0.01 mM, 0.02 mM, 0.03 mM, 0.04 mM, 0.05 mM, 0.06 mM, 0.07 mM, 0.08 mM, 0.09 mM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 15 mM, 20 mM 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

The number of extracellular particles incubated with the STING agonist may also be altered to optimize encapsulation of the STING agonist. The number of purified EV, e.g., exosome, particles may be between at least about 10⁶ to at least about 10²⁰ total particles of purified vesicles. The number of purified particles may be between about 10⁸ to 10¹⁸, 10¹⁰ to 10¹⁶, 10⁸ to 10¹⁴, or 10¹⁰ to 10¹² total particles of purified vesicles. The number of purified particles may be at least about 10⁶, 10⁸, 10¹⁰, 10¹², 10¹⁴, 10¹⁶, 10¹⁸, or 10²⁰ total particles of purified vesicles.

In some aspects, the one or more moieties can be introduced into suitable producer cells using synthetic macromolecules, such as cationic lipids and polymers (Papapetrou et al., Gene Therapy 12: S118-S130 (2005)). In some aspects, the cationic lipids form complexes with the one or more moieties through charge interactions. In some of these aspects, the positively charged complexes bind to the negatively charged cell surface and are taken up by the cell by endocytosis. In some other aspects, a cationic polymer can be used to transfect producer cells. In some of these aspects, the cationic polymer is polyethylenimine (PEI). In certain aspects, chemicals such as calcium phosphate, cyclodextrin, or polybrene, can be used to introduce the one or more moieties to the producer cells. The one or more moieties can also be introduced into a producer cell using a physical method such as particle-mediated transfection, “gene gun”, biolistics, or particle bombardment technology (Papapetrou et al., Gene Therapy 12: S118-S130 (2005)). A reporter gene such as, for example, beta-galactosidase, chloramphenicol acetyltransferase, luciferase, or green fluorescent protein can be used to assess the transfection efficiency of the producer cell.

In some aspects, the one or more moieties are introduced to the producer cell by viral transduction. A number of viruses can be used as gene transfer vehicles, including moloney murine leukemia virus (MMLV), adenovirus, adeno-associated virus (AAV), herpes simplex virus (HSV), lentiviruses, and spumaviruses. The viral mediated gene transfer vehicles comprise vectors based on DNA viruses, such as adenovirus, adeno-associated virus and herpes virus, as well as retroviral based vectors.

In some aspects, the one or more moieties are introduced to the producer cell by electroporation. Electroporation creates transient pores in the cell membrane, allowing for the introduction of various molecules into the cell. In some aspects, DNA and RNA as well as polypeptides and non-polypeptide therapeutic agents can be introduced into the producer cell by electroporation.

In some aspects, the one or more moieties are introduced to the producer cell by microinjection. In some aspects, a glass micropipette can be used to inject the one or more moieties into the producer cell at the microscopic level.

In some aspects, the one or more moieties are introduced to the producer cell by extrusion.

In some aspects, the one or more moieties are introduced to the producer cell by sonication. In some aspects, the producer cell is exposed to high intensity sound waves, causing transient disruption of the cell membrane allowing loading of the one or more moieties.

In some aspects, the one or more moieties are introduced to the producer cell by cell fusion. In some aspects, the one or more moieties are introduced by electrical cell fusion. In other aspects, polyethylene glycol (PEG) is used to fuse the producer cells. In further aspects, sendai virus is used to fuse the producer cells.

In some aspects, the one or more moieties are introduced to the producer cell by hypotonic lysis. In such aspects, the producer cell can be exposed to low ionic strength buffer causing them to burst allowing loading of the one or more moieties. In other aspects, controlled dialysis against a hypotonic solution can be used to swell the producer cell and to create pores in the producer cell membrane. The producer cell is subsequently exposed to conditions that allow resealing of the membrane.

In some aspects, the one or more moieties are introduced to the producer cell by detergent treatment. In certain aspects, producer cell is treated with a mild detergent which transiently compromises the producer cell membrane by creating pores allowing loading of the one or more moieties. After producer cells are loaded, the detergent is washed away thereby resealing the membrane.

In some aspects, the one or more moieties introduced to the producer cell by receptor mediated endocytosis. In certain aspects, producer cells have a surface receptor which upon binding of the one or more moieties induces internalization of the receptor and the associated moieties.

In some aspects, the one or more moieties are introduced to the producer cell by filtration. In certain aspects, the producer cells and the one or more moieties can be forced through a filter of pore size smaller than the producer cell causing transient disruption of the producer cell membrane and allowing the one or more moieties to enter the producer cell.

In some aspects, the producer cell is subjected to several freeze thaw cycles, resulting in cell membrane disruption allowing loading of the one or more moieties.

### V. EV Purification

The EVs, e.g., exosomes, prepared for the present disclosure can be isolated from the producer cells. It is contemplated that all known manners of isolation of EVs, e.g., exosomes, are deemed suitable for use herein. For example, physical properties of EVs, e.g., exosomes, may be employed to separate them from a medium or other source material, including separation on the basis of electrical charge (e.g., electrophoretic separation), size (e.g., filtration, molecular sieving, etc), density (e.g., regular or gradient centrifugation), Svedberg constant (e.g., sedimentation with or without external force, etc). Alternatively, or additionally, isolation may be based on one or more biological properties, and include methods that may employ surface markers (e.g., for precipitation, reversible binding to solid phase, FACS separation, specific ligand binding, non-specific ligand binding, etc.). In yet further contemplated methods, the EVs, e.g., exosomes, may also be fused using chemical and/or physical methods, including PEG-induced fusion and/or ultrasonic fusion.

The EVs, e.g., exosomes, may also be purified after incubation with the STING agonist to remove free, unencapsulated STING agonist from the composition. All manners of previously disclosed methods are also deemed suitable for use herein, including separation on the basis of physical or biological properties of EVs, e.g., exosomes.

Isolation, purification, and enrichment can be done in a general and non-selective manner (typically including serial centrifugation). Alternatively, isolation, purification, and enrichment can be done in a more specific and selective manner (e.g., using producer cell-specific surface markers). For example, specific surface markers may be used in immunoprecipitation, FACS sorting, affinity purification, bead-bound ligands for magnetic separation etc.

In some aspects, size exclusion chromatography can be utilized to isolate or purify the EVs, e.g., exosomes. Size exclusion chromatography techniques are known in the art. Exemplary, non-limiting techniques are provided herein. In some aspects, a void volume fraction is isolated and comprises the EVs, e.g., exosomes, of interest. In some aspects, for example, density gradient centrifugation can be utilized to further isolate the EVs, e.g., exosomes. Still further, in some aspects, it can be desirable to further separate the producer cell-derived EVs, e.g., exosomes, from EVs of other origin. For example, the producer cell-derived EVs, e.g., exosomes, can be separated from non-producer cell-derived EVs, e.g., exosomes, by immunosorbent capture using an antigen antibody specific for the producer cell.

In some aspects, the isolation of EVs, e.g., exosomes, may involve size exclusion chromatography or ion chromatography, such as anion exchange, cation exchange, or mixed mode chromatography. In some aspects, the isolation of EVs, e.g., exosomes, may involve desalting, dialysis, tangential flow filtration, ultrafiltration, or diafiltration, or any combination thereof0. In some aspects, the isolation of EVs, e.g., exosomes, may involve combinations of methods that include, but are not limited to, differential centrifugation, size-based membrane filtration, concentration and/or rate zonal centrifugation. In some aspects, the isolation of EVs, e.g., exosomes, may involve one or more centrifugation steps. The centrifugation may be performed at about 50,000 to 150,000×g. The centrifugation may be performed at about 50,000×g, 75,000×g, 100,000×g, 125,000×g, or 150,000×g.

### VI. Therapeutic Administration

### VI.A. Immune Modulation and Dosage

Provided herein are methods for inducing and/or modulating an immune or inflammatory response in a subject by administering a pharmaceutically effective amount of an EV, e.g., exosome, comprising a STING agonist.

Dendritic cells (DCs) are a population of antigen present cells derived from a hematopoietic cell lineage that link the innate and adaptive immune systems. DCs share a common myeloid precursor with monocytes and macrophages and are generally separated into two major groups: plasmacytoid DCs (pDCs) and myeloid DCs (mDCs), which are also known as conventional DCs (cDCs). mDCs are further classified based on their development from myeloid or lymphoid precursors and expression levels of CD8a, CD4, and C11b. A third population of DCs are monocyte-derived DCs (moDCs) which arise from a monocyte precursor, not a DC progenitor like pDCs and cDCs. moDCs develop after receiving inflammatory cues. Immature DCs reside in peripheral tissue before maturation. Several signaling pathways lead to DC maturation, including the signaling cascades induced by pattern recognition receptors (PRRs). Each subset of immature DCs varies in the protein expression patterns of PRRs which allows the immature DC populations to respond differently upon activation of the same PRR. This results in modulation of the immune response mediated by DCs. PRRs present in DCs include Toll-like receptors (TLRs), C-type lectin receptors, retinoic-acid inducible gene (RIG)-I-like receptors (RLRs), NOD-like receptors (NLRs), and STING.

The STING pathway is the dominant DNA sensing pathway in both mDCs and pDCs. Activation of the STING pathway in DCs results in Type I IFN and pro inflammatory cytokine production via TBK1, IRF3, and NF-κB signaling. Binding of IFN to their receptors on cells results in activation of IFN-stimulated response elements and the transcription of IFN-sensitive genes that result in the immune and inflammatory response. IFN signaling also cross-primes DCs to promote antigen persistence, alters the antigen repertoire available for MHCI presentation, enhances MHCI presentation of antigens, and increases the overall surface expression of MHCI, MHCII, and co-stimulatory molecules CD40, CD80, and CD86. These actions result in increased priming of tumor specific CD8+ T cells and initiation of the adaptive immune response.

In some aspects, the method of administering an EV, e.g., exosome, encapsulating a STING agonist and/or expressing a STING agonist on the surface to a subject in need thereof activates or induces dendritic cells, thereby inducing or modulating an immune or inflammatory response in the subject. In some aspects, the dendritic cells activated are myeloid dendritic cells. In some aspects, the dendritic cells are plasmacytoid dendritic cells.

In some aspects, the method induces interferon (IFN)-β production. Administration of EVs, e.g., exosomes, comprising a STING agonist (e.g., encapsulated or expressed on the luminal or exterior surface) may result in between 2-fold and 10,000-fold greater IFN-β induction compared to administration of a STING agonist alone. Administration of EVs, e.g., exosomes, comprising a STING agonist (e.g., encapsulated or expressed on the surface) may result in between about 2-5 fold, 5-10 fold, 10-20 fold, 20-30 fold, 30-40 fold, 40-50 fold, 50-60 fold, 60-70 fold, 70-80 fold, 80-90 fold, 90-100 fold, 100-200 fold, 200-300 fold, 300-400 fold, 400-500 fold, 500-600 fold, 600-700 fold, 700-800 fold, 800-900 fold, 900-1000 fold, 1000-2000 fold, 2000-3000 fold, 3000-4000 fold, 4000-5000 fold, 5000-6000 fold, 6000-7000 fold, 7000-8000 fold, 8000-9000 fold, or 9000-10,000 fold greater IFN-β induction compared to administration of a STING agonist alone. Administration of EVs, e.g., exosomes, comprising a STING agonist (e.g., encapsulated or expressed on the luminal or exterior surface) may result in greater than about 2-fold, >5 fold, >10-fold, >20-fold, >30-fold, >40-fold, >50-fold, >60-fold, >70-fold, >80-fold, >90-fold, >100-fold, >200-fold, >300-fold, >400-fold, >500-fold, >600-fold, >700-fold, >800-fold, >900-fold, >1000-fold, >2000-fold, >3000-fold, >4000-fold, >5000-fold, >6000-fold, >7000-fold, >8000-fold, >9000-fold, or >10,000-fold IFN-β induction compared to administration of a STING agonist alone. Administration of EVs, e.g., exosomes, comprising a STING agonist (e.g., encapsulated or expressed on the luminal or exterior surface) may result in between 2-fold and 10,000-fold greater IFN-β induction compared to the subject's baseline IFN-β production. Administration of EVs, e.g., exosomes, comprising a STING agonist (e.g., encapsulated or expressed on the luminal or exterior surface) may result in between about 2-5 fold, 5-10 fold, 10-20 fold, 20-30 fold, 30-40 fold, 40-50 fold, 50-60 fold, 60-70 fold, 70-80 fold, 80-90 fold, 90-100 fold, 100-200 fold, 200-300 fold, 300-400 fold, 400-500 fold, 500-600 fold, 600-700 fold, 700-800 fold, 800-900 fold, 900-1000 fold, 1000-2000 fold, 2000-3000 fold, 3000-4000 fold, 4000-5000 fold, 5000-6000 fold, 6000-7000 fold, 7000-8000 fold, 8000-9000 fold, or 9000-10,000 fold greater IFN-β induction compared to the subject's baseline IFN-β production. Administration of EVs, e.g., exosomes, comprising a STING agonist may result in greater than about 2-fold, >5 fold, >10-fold, >20-fold, >30-fold, >40-fold, >50-fold, >60-fold, >70-fold, >80-fold, >90-fold, >100-fold, >200-fold, >300-fold, >400-fold, >500-fold, >600-fold, >700-fold, >800-fold, >900-fold, >1000-fold, >2000-fold, >3000-fold, >4000-fold, >5000-fold, >6000-fold, >7000-fold, >8000-fold, >9000-fold, or >10,000-fold IFN-β induction compared to the subject's baseline IFN-β production.

In some aspects, administering an EV, e.g., exosome, disclosed herein to a subject can also regulate the levels of other immune modulators (e.g., cytokines or chemokines). In certain aspects, the method disclosed herein can increase the level of IFN-γ, CXCL9, and/or CXCL10. In some aspects, administration of EVs, e.g., exosomes, described herein (can result in between about 2-5 fold, 5-10 fold, 10-20 fold, 20-30 fold, 30-40 fold, 40-50 fold, 50-60 fold, 60-70 fold, 70-80 fold, 80-90 fold, 90-100 fold, 100-200 fold, 200-300 fold, 300-400 fold, 400-500 fold, 500-600 fold, 600-700 fold, 700-800 fold, 800-900 fold, 900-1000 fold, 1000-2000 fold, 2000-3000 fold, 3000-4000 fold, 4000-5000 fold, 5000-6000 fold, 6000-7000 fold, 7000-8000 fold, 8000-9000 fold, or 9000-10,000 fold greater amount of IFN-γ, CXCL9, and/or CXCL10 compared to a free STING agonist.

In some aspects, the method induces myeloid dendritic cell (mDC) activation. Administration of EVs, e.g., exosomes, comprising a STING agonist (e.g., encapsulated or expressed on the luminal or exterior surface) may result in between 2-fold and 50,000-fold greater mDC activation compared to administration of a STING agonist alone. Administration of EVs, e.g., exosomes, comprising a STING agonist (e.g., encapsulated or expressed on the luminal or exterior surface) may result in between about 2-5 fold, 5-10 fold, 10-20 fold, 20-30 fold, 30-40 fold, 40-50 fold, 50-60 fold, 60-70 fold, 70-80 fold, 80-90 fold, 90-100 fold, 100-200 fold, 200-300 fold, 300-400 fold, 400-500 fold, 500-600 fold, 600-700 fold, 700-800 fold, 800-900 fold, 900-1000 fold, 1000-2000 fold, 2000-3000 fold, 3000-4000 fold, 4000-5000 fold, 5000-6000 fold, 6000-7000 fold, 7000-8000 fold, 8000-9000 fold, 9000-10,000 fold, 10,000-15,000 fold, 15,000-20,000 fold, 20,000-25,000 fold, 25,000-30,000 fold, 30,000-35,000 fold, 35,000-40,000 fold, 40,000-45,000 fold, or 45,000-50,000 fold greater mDC activation compared to administration of a STING agonist alone. Administration of EVs, e.g., exosomes, comprising a STING agonist (e.g., encapsulated or expressed on the luminal or exterior surface) may result in greater than about 2-fold, >5 fold, >10-fold, >20-fold, >30-fold, >40-fold, >50-fold, >60-fold, >70-fold, >80-fold, >90-fold, >100-fold, >200-fold, >300-fold, >400-fold, >500-fold, >600-fold, >700-fold, >800-fold, >900-fold, >1000-fold, >2000-fold, >3000-fold, >4000-fold, >5000-fold, >6000-fold, >7000-fold, >8000-fold, >9000-fold, >10,000-fold, >15,000-fold, >20,000-fold, >25,000-fold, >30,000-fold, >35,000-fold, >40,000-fold, >45,000-fold, or >50,000-fold mDC activation compared to administration of a STING agonist alone.

Administration of EVs, e.g., exosomes, comprising a STING agonist (e.g., encapsulated or expressed on the luminal or exterior surface) may result in between 2-fold and 10,000-fold greater mDC activation compared to the subject's baseline mDC activation. Administration of EVs, e.g., exosomes, comprising a STING agonist (e.g., encapsulated or expressed on the luminal or exterior surface) may result in between about 2-5 fold, 5-10 fold, 10-20 fold, 20-30 fold, 30-40 fold, 40-50 fold, 50-60 fold, 60-70 fold, 70-80 fold, 80-90 fold, 90-100 fold, 100-200 fold, 200-300 fold, 300-400 fold, 400-500 fold, 500-600 fold, 600-700 fold, 700-800 fold, 800-900 fold, 900-1000 fold, 1000-2000 fold, 2000-3000 fold, 3000-4000 fold, 4000-5000 fold, 5000-6000 fold, 6000-7000 fold, 7000-8000 fold, 8000-9000 fold, 9000-10,000 fold, 10,000-15,000 fold, 15,000-20,000 fold, 20,000-25,000 fold, 25,000-30,000 fold, 30,000-35,000 fold, 35,000-40,000 fold, 40,000-45,000 fold, or 45,000-50,000 fold greater mDC activation compared to the subject's baseline mDC activation. Administration of EVs, e.g., exosomes, comprising a STING agonist (e.g., encapsulated or expressed on the luminal or exterior surface) may result in greater than about 2-fold, >5 fold, >10-fold, >20-fold, >30-fold, >40-fold, >50-fold, >60-fold, >70-fold, >80-fold, >90-fold, >100-fold, >200-fold, >300-fold, >400-fold, >500-fold, >600-fold, >700-fold, >800-fold, >900-fold, >1000-fold, >2000-fold, >3000-fold, >4000-fold, >5000-fold, >6000-fold, >7000-fold, >8000-fold, >9000-fold, >10,000-fold, >15,000-fold, >20,000-fold, >25,000-fold, >30,000-fold, >35,000-fold, >40,000-fold, >45,000-fold, or >50,000-fold mDC activation compared to the subject's baseline mDC activation.

In some aspects, the method of administering an EV, e.g., exosome, comprising a STING agonist (e.g., encapsulated or expressed on the luminal or exterior surface) does not induce monocyte activation as compared to the subject's baseline monocyte activation. In some aspects, the administration of an EV, e.g., exosome, comprising a STING agonist (e.g., encapsulated or expressed on the luminal or exterior surface) results in less than less than about 2-fold, <5 fold, <10-fold, <20-fold, <30-fold, <40-fold, <50-fold, <60-fold, <70-fold, <80-fold, <90-fold, <100-fold, <200-fold, <300-fold, <400-fold, <500-fold, <600-fold, <700-fold, <800-fold, <900-fold, <1000-fold, <2000-fold, <3000-fold, <4000-fold, <5000-fold, <6000-fold, <7000-fold, <8000-fold, <9000-fold, <10,000-fold, <15,000-fold, <20,000-fold, <25,000-fold, <30,000-fold, <35,000-fold, <40,000-fold, <45,000-fold, <50,000-fold, <55,000-fold, <60,000-fold, <65,000-fold, <70,000-fold, <75,000-fold, <80,000-fold, <85,000-fold, <90,000-fold, <95,000-fold, <100,000-fold, <200,000-fold, <300,000-fold, <400,000-fold, <500,000-fold, <600,000-fold, <700,000-fold, <800,000-fold, <900,000-fold, or <1,000,000-fold induction of monocyte activation relative to the subject's baseline monocyte activation. In some aspects, the administration of an EV, e.g., exosome, comprising a STING agonist (e.g., encapsulated or expressed on the luminal or exterior surface) to a subject results in less than about 2-5 fold, 5-10 fold, 10-20 fold, 20-30 fold, 30-40 fold, 40-50 fold, 50-60 fold, 60-70 fold, 70-80 fold, 80-90 fold, 90-100 fold, 100-200 fold, 200-300 fold, 300-400 fold, 400-500 fold, 500-600 fold, 600-700 fold, 700-800 fold, 800-900 fold, 900-1000 fold, 1000-2000 fold, 2000-3000 fold, 3000-4000 fold, 4000-5000 fold, 5000-6000 fold, 6000-7000 fold, 7000-8000 fold, 8000-9000 fold, 9000-10,000 fold, 10,000-15,000 fold, 15,000-20,000 fold, 20,000-25,000 fold, 25,000-30,000 fold, 30,000-35,000 fold, 35,000-40,000 fold, 40,000-45,000 fold, 45,000-50,000 fold, 55,000-60,000 fold, 60,000-65,000 fold, 65,000-70,000 fold, 70,000-75,000 fold, 75,000-80,000 fold, 80,000-85,000 fold, 85,000-90,000 fold, 90,000-95,000 fold, 95,000-100,000 fold, 100,000-200,000 fold, 200,000-300,000 fold, 300,000-400,000 fold, 400,000-500,000 fold, 500,000-600,000 fold, 600,000-700,000 fold, 700,000-800,000 fold, 800,000-900,000 fold, or 900,000-1,000,000 fold induction of monocyte activation relative to the subject's baseline monocyte activation.

In some aspects, the method of administering an EV, e.g., exosome, comprising a STING agonist (e.g., encapsulated or expressed on the luminal or exterior surface) to a subject does not induce monocyte activation as compared to administration of the STING agonist alone. In some aspects, the administration of an EV, e.g., exosome, comprising a STING agonist (e.g., encapsulated or expressed on the luminal or exterior surface) results in less than less than about 2-fold, <5 fold, <10-fold, <20-fold, <30-fold, <40-fold, <50-fold, <60-fold, <70-fold, <80-fold, <90-fold, <100-fold, <200-fold, <300-fold, <400-fold, <500-fold, <600-fold, <700-fold, <800-fold, <900-fold, <1000-fold, <2000-fold, <3000-fold, <4000-fold, <5000-fold, <6000-fold, <7000-fold, <8000-fold, <9000-fold, <10,000-fold, <15,000-fold, <20,000-fold, <25,000-fold, <30,000-fold, <35,000-fold, <40,000-fold, <45,000-fold, or <50,000-fold induction of monocyte activation relative to the amount of monocyte activation after administration of the free STING agonist. In some aspects, the administration of an EV, e.g., exosome, comprising a STING agonist (e.g., encapsulated or expressed on the luminal or exterior surface) to a subject results in less than about 2-5 fold, 5-10 fold, 10-20 fold, 20-30 fold, 30-40 fold, 40-50 fold, 50-60 fold, 60-70 fold, 70-80 fold, 80-90 fold, 90-100 fold, 100-200 fold, 200-300 fold, 300-400 fold, 400-500 fold, 500-600 fold, 600-700 fold, 700-800 fold, 800-900 fold, 900-1000 fold, 1000-2000 fold, 2000-3000 fold, 3000-4000 fold, 4000-5000 fold, 5000-6000 fold, 6000-7000 fold, 7000-8000 fold, 8000-9000 fold, 9000-10,000 fold, 10,000-15,000 fold, 15,000-20,000 fold, 20,000-25,000 fold, 25,000-30,000 fold, 30,000-35,000 fold, 35,000-40,000 fold, 40,000-45,000 fold, or 45,000-50,000 fold induction of monocyte activation relative to the amount of monocyte activation after administration of the free STING agonist. Monocyte activation may be measured by the surface expression of CD86 on the monocyte, or by any other appropriate monocyte activation marker known in the art.

Because of the improved therapeutic effects associated with EVs, e.g., exosomes, described herein, in some aspects, lower dosages of the EV, e.g., exosome, comprising a STING agonist (e.g., encapsulated or expressed on the luminal or exterior surface) can be delivered compared to the free STING agonist. Moreover, non-selective delivery of high doses of STING agonists can attenuate desirable immune stimulatory responses. Accordingly, because the EVs, e.g., exosomes, described herein can be administered at lower doses, in some aspects, they can operate in a wider therapeutic window and reduce the liabilities (e.g., systemic toxicity, immune cell killing, lack of cell selectivity) observed with free STING agonists.

The compositions described herein may be administered in a dosage sufficient to ameliorate the disease, disorder, condition, or symptom of the subject in need thereof. In some aspects, the dosage of the EV, e.g., exosome, comprising a STING agonist administered to a subject in need is between about 0.01 to 0.1 μM, 0.1 to 1 μM, 1 to 10 μM, 10 to 100 μM, or 100 to 1000 μM. In certain aspects, the dosage of the EV, e.g., exosome, comprising a STING agonist administered to a subject in need is about 0.01 μM, 0.05 μM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 25 μM, 30 μM, 35 μM, 40 μM, 45 μM, 40 μM, 55 μM, 60 μM, 65 μM, 70 μM, 75 μM, 80 μM, 85 μM, 90 μM, 95 μM, 100 μM, 150 μM, 200 μM, 250 μM, 300 μM, 350 μM, 400 μM, 450 μM, 500 μM, 550 μM, 600 μM, 650 μM, 700 μM, 750 μM, 800 μM, 850 μM, 900 μM, 950 μM, or 1000 μM.

In some aspects, the amount of the EV, e.g., exosome, comprising a STING agonist (e.g., encapsulated or expressed on the luminal or exterior surface) administered to a subject in need is less than 2-fold, <5 fold, <10-fold, <20-fold, <30-fold, <40-fold, <50-fold, <60-fold, <70-fold, <80-fold, <90-fold, <100-fold, <200-fold, <300-fold, <400-fold, <500-fold, <600-fold, <700-fold, <800-fold, <900-fold, <1000-fold, <2000-fold, <3000-fold, <4000-fold, <5000-fold, <6000-fold, <7000-fold, <8000-fold, <9000-fold, <10,000-fold, <15,000-fold, <20,000-fold, <25,000-fold, <30,000-fold, <35,000-fold, <40,000-fold, <45,000-fold, or <50,000-fold relative to the amount of a free STING agonist required to effect the same ameliorative results in a subject in need. In some aspects, the amount of the EV, e.g., exosome, comprising a STING agonist (e.g., encapsulated or expressed on the luminal or exterior surface) administered to a subject in need is between less than about 2-5 fold, 5-10 fold, 10-20 fold, 20-30 fold, 30-40 fold, 40-50 fold, 50-60 fold, 60-70 fold, 70-80 fold, 80-90 fold, 90-100 fold, 100-200 fold, 200-300 fold, 300-400 fold, 400-500 fold, 500-600 fold, 600-700 fold, 700-800 fold, 800-900 fold, 900-1000 fold, 1000-2000 fold, 2000-3000 fold, 3000-4000 fold, 4000-5000 fold, 5000-6000 fold, 6000-7000 fold, 7000-8000 fold, 8000-9000 fold, 9000-10,000 fold, 10,000-15,000 fold, 15,000-20,000 fold, 20,000-25,000 fold, 25,000-30,000 fold, 30,000-35,000 fold, 35,000-40,000 fold, 40,000-45,000 fold, or 45,000-50,000 fold less relative to the amount of a free STING agonist required to effect the same ameliorative results in a subject in need.

In some aspects, the method of administering an EV, e.g., exosome, comprising a STING agonist does not induce systemic inflammation as compared to the subject's baseline systemic inflammation. In some aspects, the administration of an EV, e.g., exosome, comprising a STING agonist results in less than less than about 2-fold, <5 fold, <10-fold, <20-fold, <30-fold, <40-fold, <50-fold, <60-fold, <70-fold, <80-fold, <90-fold, <100-fold, <200-fold, <300-fold, <400-fold, <500-fold, <600-fold, <700-fold, <800-fold, <900-fold, <1000-fold, <2000-fold, <3000-fold, <4000-fold, <5000-fold, <6000-fold, <7000-fold, <8000-fold, <9000-fold, <10,000-fold, <15,000-fold, <20,000-fold, <25,000-fold, <30,000-fold, <35,000-fold, <40,000-fold, <45,000-fold, or <50,000-fold induction of systemic inflammation relative to the subject's baseline systemic inflammation. In some aspects, the administration of an EV, e.g., exosome, comprising a STING agonist to a subject results in less than about 2-5 fold, 5-10 fold, 10-20 fold, 20-30 fold, 30-40 fold, 40-50 fold, 50-60 fold, 60-70 fold, 70-80 fold, 80-90 fold, 90-100 fold, 100-200 fold, 200-300 fold, 300-400 fold, 400-500 fold, 500-600 fold, 600-700 fold, 700-800 fold, 800-900 fold, 900-1000 fold, 1000-2000 fold, 2000-3000 fold, 3000-4000 fold, 4000-5000 fold, 5000-6000 fold, 6000-7000 fold, 7000-8000 fold, 8000-9000 fold, 9000-10,000 fold, 10,000-15,000 fold, 15,000-20,000 fold, 20,000-25,000 fold, 25,000-30,000 fold, 30,000-35,000 fold, 35,000-40,000 fold, 40,000-45,000 fold, or 45,000-50,000 fold induction of systemic inflammation relative to the subject's baseline systemic inflammation.

In some aspects, the method of administering an EV, e.g., exosome, comprising a STING agonist (e.g., encapsulated or expressed on the luminal or exterior surface) to a subject does not induce systemic inflammation as compared to administration of the STING agonist alone. In some aspects, the administration of an EV, e.g., exosome, comprising a STING agonist (e.g., encapsulated or expressed on the luminal or exterior surface) results in less than less than about 2-fold, <5 fold, <10-fold, <20-fold, <30-fold, <40-fold, <50-fold, <60-fold, <70-fold, <80-fold, <90-fold, <100-fold, <200-fold, <300-fold, <400-fold, <500-fold, <600-fold, <700-fold, <800-fold, <900-fold, <1000-fold, <2000-fold, <3000-fold, <4000-fold, <5000-fold, <6000-fold, <7000-fold, <8000-fold, <9000-fold, <10,000-fold, <15,000-fold, <20,000-fold, <25,000-fold, <30,000-fold, <35,000-fold, <40,000-fold, <45,000-fold, or <50,000-fold induction of systemic inflammation relative to the amount of systemic inflammation after administration of the free STING agonist. In some aspects, the administration of an EV, e.g., exosome, comprising a STING agonist (e.g., encapsulated or expressed on the luminal or exterior surface) to subject results in less than about 2-5 fold, 5-10 fold, 10-20 fold, 20-30 fold, 30-40 fold, 40-50 fold, 50-60 fold, 60-70 fold, 70-80 fold, 80-90 fold, 90-100 fold, 100-200 fold, 200-300 fold, 300-400 fold, 400-500 fold, 500-600 fold, 600-700 fold, 700-800 fold, 800-900 fold, 900-1000 fold, 1000-2000 fold, 2000-3000 fold, 3000-4000 fold, 4000-5000 fold, 5000-6000 fold, 6000-7000 fold, 7000-8000 fold, 8000-9000 fold, 9000-10,000 fold, 10,000-15,000 fold, 15,000-20,000 fold, 20,000-25,000 fold, 25,000-30,000 fold, 30,000-35,000 fold, 35,000-40,000 fold, 40,000-45,000 fold, or 45,000-50,000 fold induction of systemic inflammation relative to the amount of systemic inflammation after administration of the free STING agonist. Systemic inflammation may be quantified or measured by any appropriate method known in the art.

In some aspects, the method of administering an EV, e.g., exosome, comprising a STING agonist (e.g., encapsulated or expressed on the luminal or exterior surface) to a subject additionally comprises administering an additional therapeutic agent. In some aspects, the EV further comprises an additional therapeutic agent. In some aspects, the additional therapeutic agent comprises a ligand, a cytokine, or an antibody In some aspects, the additional therapeutic agent is an immunomodulating agent. In some aspects, the immunomodulating component is an inhibitor for a negative checkpoint regulator or an inhibitor for a binding partner of a negative checkpoint regulator. In some of these aspects, the negative checkpoint regulator is selected from the group consisting of: cytotoxic T-lymphocyte-associated protein 4 (CTLA-4), programmed cell death protein 1 (PD-1), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, and CD73. In various aspects, the additional therapeutic agent is an antibody or antigen-binding fragment thereof. In some aspects, the antibody or antigen-binding fragment thereof is one or more whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. The term antibody includes bispecific antibodies and multispecific antibodies so long as they exhibit the desired biological activity or function. In some aspects, the additional therapeutic agent is a therapeutic antibody or antigen-binding fragment thereof that is an inhibitor of CTLA-4, PD-1, PD-L1, PD-L2, TIM-3, or LAG3.

In some aspects, the additional therapeutic agent is an agent that prevents or treats T cell exhaustion. Such agents may increase, decrease, or modulate the expression of genes associated with T cell exhaustion, including Prdm1, Bhlhe40, Irf4, Ikzf2, Zeb2, Lass6, Egr2, Tox, Eomes, Nfatc1, Nfatc2, Zbtb32, Rbpj, Hif1a, Lag3, Tnfrsf9, Ptger2, Havcr2, Alcam, Tigit, Ctla4, Ptger4, Tnfrsf1b, Ccl4, CD109, CD200, Tnfsf9, Nrp1, Sema4c, Ptprj, Il21, Tspan2, Rgs16, Sh2d2a, Nucb1, Plscr1, Ptpn11, Prkca, Plscr4, Casp3, Gpd2, Gas2, Sh3rf1, Nhedc2, Plek, Tnfaip2, and Ctsb, or any combination thereof. Therapeutic agents may also increase, decrease, or modulate a protein associated with T cell exhaustion, including NFAT-1 or NFAT-2.

### VI.B. Method of Treating Cancer

Provided herein are methods of treating cancer in a subject. The method comprises administering to the subject a therapeutically effective amount of the compositions disclosed herein, wherein the composition is capable of up-regulating a STING-mediated immune response in the subject, thereby enhancing the tumor targeting of the subject's immune system. In some aspects, the composition is administered intra-tumorally to the subject. In some aspects, the composition is administered parenterally, orally, intravenously, intramuscularly, intraperitoneally, or via any other appropriate administration route.

Also provided herein are methods of preventing metastasis of cancer in a subject. The method comprises administering to the subject a therapeutically effective amount of the compositions disclosed herein, wherein the composition is capable of preventing one or more tumors at one location in the subject from promoting the growth of one or more tumors at another location in the subject. In some aspects, the composition is administered intratumorally in a first tumor in one location, and the composition administered in a first tumor prevents metastasis of one or more tumors at a second location.

In some aspects, administering an EV, e.g., exosome, disclosed herein inhibits and/or reduces tumor growth in a subject. In some aspects, the tumor growth (e.g., tumor volume or weight) is reduced by at least about 5%, at least about 10%, at least about 20%, at least about 30%, at least about 40%, at least about 50%, at least about 60%, at least about 70%, at least about 80%, at least about 90%, or about 100% compared to a reference (e.g., tumor volume in a corresponding subject after administration of free STING agonist or an EV, e.g., exosome, without the STING agonist).

In some aspects, the cancer being treated is characterized by infiltration of leukocytes (T-cells, B-cells, macrophages, dendritic cells, monocytes) into the tumor microenvironment, or so-called “hot tumors” or “inflammatory tumors”. In some aspects, the cancer being treated is characterized by low levels or undetectable levels of leukocyte infiltration into the tumor microenvironment, or so-called “cold tumors” or “non-inflammatory tumors”. In some aspects, an EV, e.g., exosome, is administered in an amount and for a time sufficient to convert a “cold tumor” into a “hot tumor”, i.e., said administering results in the infiltration of leukocytes (such as T-cells) into the tumor microenvironment. In certain aspects, cancer comprises bladder cancer, cervical cancer, renal cell cancer, testicular cancer, colorectal cancer, lung cancer, head and neck cancer, and ovarian, lymphoma, liver cancer, glioblastoma, melanoma, myeloma, leukemia, pancreatic cancers, or combinations thereof. The term “distal tumor”, “distant tumor”, or “secondary tumor” as used herein refers to a tumor that has spread from the original (or primary) tumor to distant organs or distant tissues, e.g., lymph nodes. In some aspects, the EVs, e.g., exosomes, of the disclosure treats a tumor after the metastatic spread.

Non-limiting examples of cancers (or tumors) that can be treated with methods disclosed herein include squamous cell carcinoma, small-cell lung cancer (SCLC), non-small cell lung cancer, squamous non-small cell lung cancer (NSCLC), nonsquamous NSCLC, gastrointestinal cancer, renal cancer (e.g., clear cell carcinoma), ovarian cancer, liver cancer (e.g., hepatocellular carcinoma), colorectal cancer, endometrial cancer, kidney cancer (e.g., renal cell carcinoma (RCC)), prostate cancer (e.g., hormone refractory prostate adenocarcinoma), thyroid cancer, pancreatic cancer, cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer (e.g., choriocarcinoma and non-seminoma), carcinoma of the fallopian tubes, carcinoma of the endometrium, carcinoma of the cervix, carcinoma of the vagina, carcinoma of the vulva, cancer of the esophagus (e.g., gastroesophageal junction cancer), cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, tumor angiogenesis, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki1⁺) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyosarcoma; seminoma, teratocarcinoma, tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, and any combinations thereof.

In some aspects, a cancer (or tumor) that can be treated comprises a breast cancer, head and neck cancer, uterine cancer, brain cancer, skin cancer, renal cancer, lung cancer, colorectal cancer, prostate cancer, liver cancer, bladder cancer, kidney cancer, peritoneal cancer, pancreatic cancer, thyroid cancer, esophageal cancer, eye cancer, stomach (gastric) cancer, gastrointestinal cancer, carcinoma, sarcoma, leukemia, lymphoma, myeloma, or a combination thereof. In certain aspects, a cancer that can be treated with the present disclosure is a pancreatic cancer and/or a peritoneal cancer.

In some aspects, the methods described herein can also be used for treatment of metastatic cancers, unresectable, refractory cancers (e.g., cancers refractory to previous cancer therapy), and/or recurrent cancers.

In some aspects, EVs, e.g., exosomes, disclosed herein can be used in combination with one or more additional anti-cancer and/or immunomodulating agents. Such agents can include, for example, chemotherapy drugs, small molecule drugs, or antibodies that stimulate the immune response to a given cancer. In some aspects, the methods described herein are used in combination with a standard of care treatment (e.g., surgery, radiation, and chemotherapy).

In some aspects, a method for treating a cancer disclosed herein can comprise administering (i) an EV, e.g., exosome, comprising a STING agonist (e.g., encapsulated or expressed on the luminal or exterior surface) in combination with (ii) an IL-12 moiety with an anti-cancer agent, e.g., one or more immuno-oncology agents, e.g., a checkpoint inhibitor, such that multiple elements of the immune pathway can be targeted. In some aspects, a method for treating a cancer disclosed herein comprises administering (i) a first EV, e.g., exosome, comprising a STING agonist (e.g., encapsulated or expressed on the luminal or exterior surface), (ii) a second EV, e.g., exosome, comprising an IL-12 moiety (e.g., encapsulated or expressed on the luminal or exterior surface), and (iii) an anti-cancer agent, e.g., one or more immuno-oncology agents, e.g., a checkpoint inhibitor, such that multiple elements of the immune pathway can be targeted. In some aspects, a method for treating a cancer disclosed herein comprises administering (i) a first EV, e.g., exosome, comprising a STING agonist (e.g., encapsulated or expressed on the luminal or exterior surface); (ii) an IL-12 moiety; and (iii) a second EV, e.g., exosome, comprising an anti-cancer agent, e.g., one or more immuno-oncology agents, e.g., a checkpoint inhibitor, wherein the anti-cancer agent is encapsulated or expressed on the luminal or exterior surface of the second EV, e.g., exosome. In some aspects, a method for treating a cancer disclosed herein comprises administering (i) a STING agonist; (ii) a first EV, e.g., exosome, comprising an IL-12 moiety (e.g., encapsulated or expressed on the luminal or exterior surface); and (iii) a second EV, e.g., exosome, comprising an anti-cancer agent, e.g., one or more immuno-oncology agents, e.g., a checkpoint inhibitor, wherein the anti-cancer agent is encapsulated or expressed on the luminal or exterior surface of the second EV, e.g., exosome. In some aspects, a method for treating a cancer disclosed herein comprises administering (i) a first EV, e.g., exosome, comprising a STING agonist (e.g., encapsulated or expressed on the luminal or exterior surface), (ii) a second EV, e.g., exosome, comprising an IL-12 moiety (e.g., encapsulated or expressed on the luminal or exterior surface), and (iii) a third EV, e.g., exosome, comprising an anti-cancer agent (e.g., one or more immuno-oncology agents, e.g., a checkpoint inhibitor), wherein the anti-cancer agent is encapsulated or expressed on the luminal or exterior surface of the third EV, e.g., exosome.

In some aspects, a method for treating a cancer disclosed herein comprises administering (i) an EV, e.g., exosome, comprising (a) a STING agonist (e.g., encapsulated or expressed on the luminal or exterior surface) and (b) an IL-12 moiety (e.g., encapsulated or expressed on the luminal or exterior surface); and (ii) an anti-cancer agent, e.g., one or more immuno-oncology agents, e.g., a checkpoint inhibitor, such that multiple elements of the immune pathway can be targeted. In some aspects, a method for treating a cancer disclosed herein comprises administering (i) an EV, e.g., exosome, comprising (a) a STING agonist (e.g., encapsulated or expressed on the luminal or exterior surface) and (b) an anti-cancer agent, e.g., one or more immuno-oncology agents, e.g., a checkpoint inhibitor (e.g., encapsulated or expressed on the luminal or exterior surface); and (ii) an IL-12 moiety. In some aspects, a method for treating a cancer disclosed herein comprises administering (i) an EV, e.g., exosome, comprising (a) an IL-12 moiety (e.g., encapsulated or expressed on the luminal or exterior surface) and (b) an anti-cancer agent, e.g., one or more immuno-oncology agents, e.g., a checkpoint inhibitor (e.g., encapsulated or expressed on the luminal or exterior surface); and (ii) a STING agonist.

In some aspects, a method for treating a cancer disclosed herein comprises administering (i) an EV, e.g., exosome, comprising a STING agonist (e.g., encapsulated or expressed on the luminal or exterior surface); (ii) an IL-12 moiety; and (iii) an anti-cancer agent, e.g., one or more immuno-oncology agents, e.g., a checkpoint inhibitor, such that multiple elements of the immune pathway can be targeted. In some aspects, a method for treating a cancer disclosed herein comprises administering (i) an EV, e.g., exosome, comprising an IL-12 moiety (e.g., encapsulated or expressed on the luminal or exterior surface); (ii) a STING agonist; and (iii) an anti-cancer agent, e.g., one or more immuno-oncology agents, e.g., a checkpoint inhibitor. In some aspects, a method for treating a cancer disclosed herein comprises administering (i) an EV, e.g., exosome, comprising an anti-cancer agent, e.g., one or more immuno-oncology agents, e.g., a checkpoint inhibitor (e.g., encapsulated or expressed on the luminal or exterior surface); (ii) a STING agonist; and (iii) an IL-12 moiety.

In some aspects, the method comprising administering (i) a STING agonist (e.g., associated with an EV (e.g. exosome) or a free STING agonist) prior to administering (ii) an IL-12 moiety (e.g., associated with an EV (e.g. exosome) or a free IL-12 moiety). In some aspects, the method comprising administering (i) an IL-12 moiety (e.g., associated with an EV (e.g. exosome) or a free IL-12 moiety) prior to administering (ii) a STING agonist (e.g., associated with an EV (e.g. exosome) or a free STING agonist). In some aspects, (i) is administered at least about 1 hour, at least about 2 hours, at least about 3 hours, at least about 6 hours, at least about 12 hours, at least about 18 hours, at least about 24 hours, at least about 36 hours, at least about 48 hours, at least about 72 hours, or at least about 96 hours prior to (ii). In some aspects, (i) is administered at least about 24 hours before (ii). In some aspects, (i) is administered at least about 48 hours before (ii).

In some aspects, the method comprising administering (i) a STING agonist (e.g., associated with an EV (e.g. exosome) or a free STING agonist), (ii) an IL-12 moiety (e.g., associated with an EV (e.g. exosome) or a free IL-12 moiety), and (iii) an additional anti-cancer agent (e.g., associated with an EV (e.g. exosome) or not associated with an EV (e.g. exosome)), wherein (a) (i) is administered before (ii), and wherein (ii) is administered before (iii); (b) (ii) is administered before (i), and wherein (i) is administered before (iii); (c) (iii) is administered before (i), and wherein (i) is administered before (ii); (d) (iii) is administered before (ii), and wherein (ii) is administered before (ii); (e) (i) and (ii) are administered concurrently and before (iii); (f) (i) and (ii) are administered concurrently and after (iii); (g) (i) and (iii) are administered concurrently and before (ii); (h) (i) and (iii) are administered concurrently and after (ii); (j) (ii) and (iii) are administered concurrently and before (i); (k) (ii) and (iii) are administered concurrently and after (i); or (1) (i), (ii), and (iii) are administered concurrently. In some aspects, administration is separated by at least about 1 hour, at least about 2 hours, at least about 3 hours, at least about 6 hours, at least about 12 hours, at least about 18 hours, at least about 24 hours, at least about 36 hours, at least about 48 hours, at least about 72 hours, or at least about 96 hours prior to (ii).

Non-limiting examples of such combinations include: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxygenase (IDO), dioxygenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immuno repressive cytokines.

In some aspects, an immuno-oncology agent that can be used in combination with EVs, e.g., exosomes, disclosed herein and an IL-12 moiety further comprises a checkpoint inhibitor (i.e., blocks signaling through the particular immune checkpoint pathway). Non-limiting examples of checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof.

In some aspects, an immuno-oncology agent comprises an immune checkpoint activator (i.e., promotes signaling through the particular immune checkpoint pathway). In certain aspects, immune checkpoint activator comprises OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), or any combination thereof.

In some aspects, a combination of an EV, e.g., exosome, disclosed herein and a second agent discussed herein (e.g., immune checkpoint inhibitor) can be administered concurrently as a single composition in a pharmaceutically acceptable carrier. In other aspects, a combination of an EV, e.g., exosome, and a second agent discussed herein (e.g., immune checkpoint inhibitor) can be administered concurrently as separate compositions. In further aspects, a combination of an EV, e.g., exosome, and a second agent discussed herein (e.g., immune checkpoint inhibitor) can be administered sequentially. In some aspects, an EV, e.g., exosome, is administered prior to the administration of a second agent (e.g., immune checkpoint inhibitor).

### VLC. Pharmaceutical Compositions

Provided herein are pharmaceutical compositions comprising EVs, e.g., exosomes, that are suitable for administration to a subject. The pharmaceutical compositions generally comprise a plurality of EVs, e.g., exosomes, comprising a STING agonist and/or an IL-12 moiety (e.g., encapsulated or expressed on the luminal or exterior surface) and a pharmaceutically-acceptable excipient or carrier in a form suitable for administration to a subject. In some aspects, the pharmaceutical composition comprises (i) a plurality of EVs, e.g., exosomes, comprising a STING agonist (e.g., encapsulated or expressed on the luminal or exterior surface), (ii) an IL-12 moiety, and (iii) a pharmaceutically-acceptable excipient or carrier in a form suitable for administration to a subject. In some aspects, the pharmaceutical composition comprises (i) a plurality of EVs, e.g., exosomes, comprising an IL-12 moiety (e.g., encapsulated or expressed on the luminal or exterior surface), (ii) a STING agonist, and (iii) a pharmaceutically-acceptable excipient or carrier in a form suitable for administration to a subject. In some aspects, the pharmaceutical composition comprises (i) a plurality of EVs, e.g., exosomes, comprising a STING agonist (e.g., encapsulated or expressed on the luminal or exterior surface), (ii) a plurality of EVs, e.g., exosomes, comprising an IL-12 moiety (e.g., encapsulated or expressed on the luminal or exterior surface), and (iii) a pharmaceutically-acceptable excipient or carrier in a form suitable for administration to a subject. In some aspects, the pharmaceutical composition comprises (i) a plurality of EVs, e.g., exosomes, comprising a STING agonist and an IL-12 moiety (e.g., encapsulated or expressed on the luminal or exterior surface), and (ii) a pharmaceutically-acceptable excipient or carrier in a form suitable for administration to a subject.

Pharmaceutically-acceptable excipients or carriers are determined in part by the particular composition being administered, as well as by the particular method used to administer the composition. Accordingly, there is a wide variety of suitable formulations of pharmaceutical compositions comprising a plurality of EVs, e.g., exosomes, (See, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration.

In some aspects, the pharmaceutical composition comprises one or more STING agonist and the EVs, e.g., exosomes, described herein. The EVs of the present disclosure, e.g., EV comprising a STING agonist and second EV comprising an IL-12 moiety, can be formulated together or separate.

Pharmaceutically-acceptable excipients include excipients that are generally safe (GRAS), non-toxic, and desirable, including excipients that are acceptable for veterinary use as well as for human pharmaceutical use.

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the EVs, e.g., exosomes, described herein, use thereof in the compositions is contemplated. Supplementary therapeutic agents may also be incorporated into the compositions. Typically, a pharmaceutical composition is formulated to be compatible with its intended route of administration. The EVs, e.g., exosomes, can be administered by intratumoral, parenteral, topical, intravenous, oral, subcutaneous, intraarterial, intradermal, transdermal, rectal, intracranial, intraperitoneal, intranasal; intramuscular route or as inhalants. In one aspect, the pharmaceutical composition comprising EVs, e.g., exosomes, is administered intravenously, e.g. by injection. The EVs, e.g., exosomes, can optionally be administered in combination with other therapeutic agents that are at least partly effective in treating the disease, disorder or condition for which the EVs, e.g., exosomes, are intended.

Solutions or suspensions can include the following components: a sterile diluent such as water, saline solution, fixed oils, polyethylene glycols, glycerin, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs, e.g., exosomes, in an effective amount and in an appropriate solvent with one or a combination of ingredients enumerated herein, as desired. Generally, dispersions are prepared by incorporating the EVs, e.g., exosomes, into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The EVs, e.g., exosomes, can be administered in the form of a depot injection or implant preparation which can be formulated in such a manner to permit a sustained or pulsatile release of the EVs, e.g., exosomes.

Systemic administration of compositions comprising EVs, e.g., exosomes, can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the modified EVs, e.g., exosomes, are formulated into ointments, salves, gels, or creams as generally known in the art.

### EXAMPLES

The following examples are provided for illustrative purposes only, and are not to be construed as limiting the scope or content of the invention in any way. The practice of the current invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); Green & Sambrook et al., Molecular Cloning: A Laboratory Manual, 4th Edition (Cold Spring Harbor Laboratory Press, 2012); Colowick & Kaplan, Methods In Enzymology (Academic Press); Remington: The Science and Practice of Pharmacy, 22nd Edition (Pharmaceutical Press, 2012); Sundberg & Carey, Advanced Organic Chemistry: Parts A and B, 5th Edition (Springer, 2007).

### Methods

### Exosome Purification

HEK293 SF cells were grown to high density in chemically defined medium for 7 days. Conditioned cell culture media was collected and centrifuged at 300-800×g for 5 minutes at room temperature to remove cells and large debris. Media supernatant was then supplemented with 1000 U/L BENZONASE® and incubated at 37° C. for 1 hour in a water bath. Supernatant was collected and centrifuged at 16,000×g for 30 minutes at 4° C. to remove residual cell debris and other large contaminants. Supernatant was then ultracentrifuged at 133,900×g for 3 hours at 4° C. to pellet the exosomes. Supernatant was discarded and any residual media was aspirated from the bottom of the tube. The pellet was resuspended in 200-1000 μL PBS (—Ca —Mg).

To further enrich exosome populations, the pellet was processed via density gradient purification (sucrose or OPTIPREP™). For sucrose gradient purification, the exosome pellet was layered on top of a sucrose gradient as defined in Table 5 below.

TABLE 5

| WORKING | 65% | |
|---|---|---|
| PERCENTAGE | STOCK | MILLI-Q |
| (%) | VOL. (ML) | VOL. (ML) |
| 50 | 3.85 | 1.15 |
| 40 | 3.08 | 1.92 |
| 25 | 1.92 | 3.08 |
| 10 | 0.46 | 2.54 |

The gradient was spun at 200,000×g for 16 hours at 4° C. in a 12 mL Ultra-Clear (344059) tube placed in a SW 41 Ti rotor to separate the exosome fraction.

The exosome layer was gently removed from the top layer and diluted in ˜32.5 mL PBS in a 38.5 mL Ultra-Clear (344058) tube and ultracentrifuged again at 133,900×g for 3 hours at 4° C. to pellet the purified exosomes. The resulting pellet was resuspended in a minimal volume of PBS (˜200 μL) and stored at 4° C.

For OPTIPREP™ gradient, a 3-tier sterile gradient is prepared with equal volumes of 10%, 30%, and 45% OPTIPREP™ in a 12 mL Ultra-Clear (344059) tube for a SW 41 Ti rotor. The pellet was added to the OPTIPREP™ gradient and ultracentrifuged at 200,000×g for 16 hours at 4° C. to separate the exosome fraction. The exosome layer was then gently collected from the top ˜3 mL of the tube.

The exosome fraction was diluted in ˜32 mL PBS in a 38.5 mL Ultra-Clear (344058) tube and ultracentrifuged at 133,900×g for 3 hours at 4° C. to pellet the purified exosomes. The pelleted exosomes were then resuspended in a minimal volume of PBS (˜200 μL) and store at 4° C.

In Vivo Intratumoral Microinjection Studies with CIVO®

### Tumor Cell Culture

A20 cells (ATCC Lot #70006082) were cultured in RPMI 1640 with L-Glutamine (ThermoFisher), 10% fetal bovine serum (Thermofisher) and 50 nanomolar BME at 37 degrees Celsius, 5% C02. IMPACT III testing (IDEXX Bioresearch) was carried out to confirm mycoplasma- and pathogen-free status. Cells were expanded and cryopreserved following 2-3 passages after obtaining from vendor. After thawing, cells were maintained for a maximum of 8 weeks by sub-culturing 3 times a week and replenished from a fresh frozen stock thereafter.

### In Vivo Studies

All experiments in mice were approved by IACUC Board of Presage Biosciences, Seattle, Wash. (Protocol number PR-001) and were performed at Presage in accordance with relevant guidelines and regulations. All relevant procedures were performed under anesthesia and all efforts were made to minimize pain and suffering. Female BALB/cAnNHsd mice (Envigo) with an average weight of 18 gm were used for experiments at 5-7 weeks of age. For generating A20 allografts, mice were inoculated with 1 million A20 cells in 100 μl inoculation volume.

### CIVO® Intra-Tumoral Microinjections

CIVO intra-tumoral microinjections were performed as described in Klinghoffer et al. (2016) Science Translational Medicine. Briefly, mice (n=6 per time point, 4 and 24 hours) were enrolled in microinjection studies when implanted tumors reached the following approximate dimensions: 14 mm (length), 10 mm (width) and 7 mm (depth). The CIVO device was configured with 6 thirty-gauge injection needles with a total volume delivery of 2.0 μl. Presage's fluorescent tracking marker (FTM, 5% by volume) was added to the injection contents for spatial orientation. Agents microinjected were as follows: control PTGFRN++ GFP exosomes, ML RR-S2 CDA loaded PTGFRN++ GFP Exosomes, ML RR-S2 CDA loaded PTGFRN++GFP Desilylated exosomes, ML RR-S2 CDA loaded native exosomes, all at 10 ng/μl ML RR-S2 CDA such that the total amount delivered was 20 ng. Free ML RR-S2 CDA was microinjected at both 20 ng and 2 μg. At 4 and 24 hours following CIVO microinjections, mice were euthanized using C02 inhalation for biomarker analyses.

### Histology, Immunohistochemistry and In Situ Hybridization

Resected tumors were cut into 2 mm thick sections perpendicular to the injection columns, fixed in 10% buffered formalin for 48 hours. UV imaging was used to confirm CIVO microinjections based on signal from the FTM injected at each CIVO site. 2 mm-thick tissue sections were then processed for standard paraffin embedding. 4 μm thick sections were for used for all histological assays as described below. Hematoxylin-Eosin (H&E) staining was performed using standard methods.

### Immunohistochemistry

Formalin-fixed, paraffin embedded tumors were cut onto slides with a thickness of 4 μm. Slides were baked for 1 hour at 60° C., deparaffinized in xylene, and rehydrated via graded alcohols.

Slides underwent a 20-minute target retrieval solution incubation at 100° C., followed by a 20-minute cool down to room temperature. Serum Block (5% Normal Goat Serum in TBST) was performed for 1 hr at room temperature. Primary antibody staining was carried out with appropriate primary antibody in 5% NGS TBS diluent overnight at room temperature. Corresponding isotype controls were included in each batch. Secondary antibody staining was carried out with appropriate secondary antibody in 5% NGS TBS diluent overnight at room temperature. The slides were counterstained with DAPI for 10 minutes and coverslipped with Prolong Gold mounting medium (Invitrogen). Stained slides were imaged using a digital, automated, high resolution scanner.

In situ hybridization was completed using the RNAscope multiplex fluorescent reagent kit v2 (Advanced Cell Diagnostics). Formalin-fixed, paraffin embedded tumors were cut onto slides with a thickness of 4 μm. Slides were baked for 1 hour at 60° C., deparaffinized in xylene, and rehydrated via graded alcohols. Hydrogen peroxide was added for 10 minutes to quench endogenous peroxidase activity. Slides underwent a 15-minute target retrieval solution incubation at 100° C., followed by a 15-minute protease digestion at 40° C. The RNAscope ISH assay was completed with a mouse Ifnb1 probe (Advanced Cell Diagnostics) and TSA Plus Cyanine 5 detection (Perkin Elmer). The slides were counterstained with DAPI for 10 minutes and coverslipped with Prolong Gold mounting medium (Invitrogen). Stained slides were imaged using a digital, automated, high resolution scanner.

### Whole-Slide Scanning and Image Analysis

Images of every cell from each tissue section stained were captured by digital, automated, high-resolution whole-tissue scanning (3D Histech Panoramic 250 Flash). Tumor responses were quantified from image files from each tissue section using Presage's custom CIVO Analyzer image analysis platform. Whole-tissue section images captured by the slide scanners were automatically processed by CIVO Analyzer. Each cell from each tissue section was segmented based on the nuclear (DAPI) signal and classified as biomarker-negative or -positive using Cell Profiler (Broad Institute). Following cellular segmentation and classification, circular regions of interest (ROI) were localized around each microinjection site in each image around the FTM at each position, with the largest ROI no greater than 2000 μm in radius. In order to mitigate the influence of pre-existing necrosis on biomarker measurements, injection sites that fall within largely acellular tumor regions are excluded prior to quantitative analysis.

### Example 1: Exosome-Encapsulated STING Agonists

Encapsulation of STING agonist

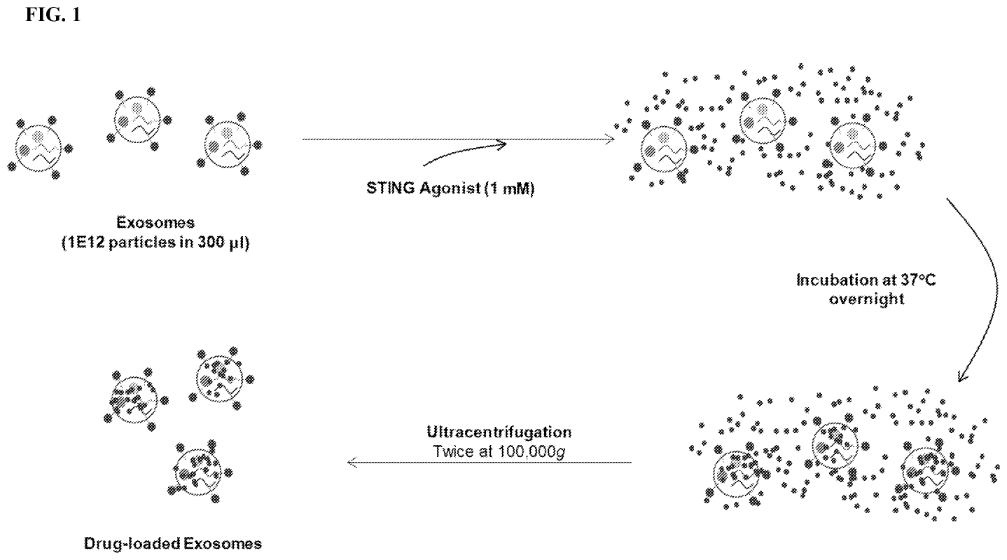

1 mM STING agonist including ML RR-S2 CDA ammonium salt (MedChem Express, Cat. No. HY-12885B) and (3-3 cAIMPdFSH; InvivoGen, Cat. No. tlrl-nacairs) was incubated with purified exosomes (1E12 total particles) in 300 ul of PBS at 37° C. overnight. The mixture was then washed twice in PBS and purified by ultra-centrifugation at 100,000×g (FIG. 1).

### Quantification of the Cyclic Dinucleotide STING Agonist

### Sample Preparation for LC-MS Analysis

All samples were received in either phosphate-buffered saline (PBS) buffer or PBS and 5% sucrose. Prior to analysis, the particle concentration (P/mL) was measured by Nanoparticle Tracking Analysis (NTA) on the NanoSight NS300. All standards and samples were prepared such that each injection contained a virtually identical number of particles. This was achieved through a combination of diluting samples and spiking exosomes into standards to reach a final concentration of 1.0-4.0 E+11 P/mL, depending on the initial particle concentrations of the samples.

Standard curves were prepared by spiking a known concentration of STING agonist into PBS buffer, then preparing additional standards through serial dilution. Separate standards were typically prepared such that the final concentrations (after all sample preparation steps) were 25, 50, 250, 500, 1250, 2500, and 5000 nM STING agonist. First, 75.0 μL of each appropriately diluted sample and each matrix-matched standard was prepared in a separate 1.5 mL microcentrifuge tube. Next, 25.0 μL of exosome lysis buffer (60 mM Tris, 400 mM GdmCl, 100 mM EDTA, 20 mM TCEP, 1.0% Triton X-100) was added to each tube, then all tubes were vortexed to mix and briefly centrifuged to settle. Finally, 1.0 μL of concentrated Proteinase K enzyme solution (Dako, reference S3004) was added to each tube, and again all tubes were vortexed and then briefly centrifuged, followed by incubation at 55° C. for 60 minutes. Prior to injection on the LC-MS, samples were allowed to cool to room temperature and were transferred to HPLC vials.

### LC-MS Analysis

20.0 μL of standards and samples were injected neat into an UltiMate 3000 RSCLnano (Thermo Fisher Scientific) low flow chromatography system without cleanup. Separation of analytes was performed using a Phenomenex Kinetex EVO C18 core-shell analytical column (50×2.1 mm, 2.6 μm particle size, 100 Å pore size) and the loading pumps delivering a gradient of mobile phase A (MPA:water, 0.1% formic acid) and mobile phase B (MPB:acetonitrile, 0.1% formic acid) at a flowrate of 500 μL/min. The gradient began at 2% MPB, which was held for 2 minutes to load and desalt the STING agonist analyte. The percentage MPB then increased from 2-30% over 3 minutes to elute the STING agonist analyte. The percentage MPB then increased from 30-95% over 1 minute, held at 95% for 3 minutes, decreased from 95-2% over 1 minute, and then held at 2% for another 3 minutes to re-equilibrate the column. The total runtime for the method was 13 minutes, and LC flow was only directed into the MS between 2.5-4.5 minutes. Typical carry-over was less than 0.05% of the peak area of the previous injection, therefore blank injections were not performed between analytical injections.

Mass analyses were performed with a Q Exactive Basic (Thermo Fisher Scientific) mass spectrometer with the Ion Max source and a HESI-II probe operating in negative ion mode, and mass spectra were collected using Full MS-SIM mode scanning from 500-800 Da with an AGC target of 1E+6 ions, a maximum injection time of 200 ms, and a resolution of 35,000. STING agonist quantitation was performed using the monoisotopic-1 STING agonist peak by selectively extracting all ions within the m/z range from 688.97-689.13 Da, and then integrating the resulting peak at a retention time between 3.80-3.90 minutes. The concentration of STING agonist in a given sample was determined by comparing the STING agonist peak area in that sample to STING agonist peak areas generated by standards, which is typical of relative quantitation.

### Example 2: In Vivo Administration of Exosomes with Surface-Display of IL-12 in Combination with Exosome-Loaded STING Agonists

To determine if there is any synergy with exosomes with surface-display of IL-12 and exosome-loaded STING agonist, B16F10 mice were subcutaneously inoculated with tumor cells at two different sites (“primary” and “secondary”). At day 4 post inoculation, at which point the mice had developed primary and secondary tumors of about 50 mm³, mice were divided into 7 treatment groups (Table 6). Mice in group 1 were administered PBS by intratumoral (IT) injection (of the primary tumor) on days 4, 6, 7, 8, and 10 post inoculation, and 10 mg/kg of an isotype control by intraperitoneal (IP) injection on days 4, 7, 11, and 14 post inoculation. Mice in group 2 were administered 10 ng of exosomes loaded with STING agonist by IT injection (of the primary tumor) on days 4, 7, and 10 post inoculation, and 10 mg/kg of an isotype control by intraperitoneal (IP) injection on days 4, 7, 11, and 14 post inoculation. Mice in group 3 were administered 10 ng of exosomes loaded with STING agonist by IT injection (of the primary tumor) on days 4, 7, and 10 post inoculation, and 10 mg/kg of an anti-PD-1 antibody (aPD-1) by IP injection on days 4, 7, 11, and 14 post inoculation. Mice in group 4 were administered 100 ng of exosomes loaded with IL-12 by IT injection (of the primary tumor) on days 4, 6, and 8 post inoculation, and 10 mg/kg of an isotype control by IP injection on days 4, 7, 11, and 14 post inoculation. Mice in group 5 were administered 100 ng of exosomes loaded with IL-12 by IT injection (of the primary tumor) on days 4, 6, and 8 post inoculation, and 10 mg/kg of an anti-PD-1 antibody (aPD-1) by IP injection on days 4, 7, 11, and 14 post inoculation. Mice in group 6 were administered 10 ng of exosomes loaded with STING agonist by IT injection (of the primary tumor) on days 4, 7, and 10 post inoculation; 100 ng of exosomes loaded with IL-12 by IT injection (of the primary tumor) on days 6, 8, and 10 post inoculation; and 10 mg/kg of an isotype control by IP injection on days 4, 7, 11, and 14 post inoculation. Mice in group 7 were administered 10 ng of exosomes loaded with STING agonist by IT injection (of the primary tumor) on days 4, 7, and 10 post inoculation; 100 ng of exosomes loaded with IL-12 by IT injection (of the primary tumor) on days 6, 8, and 10 post inoculation; and 10 mg/kg of an anti-PD-1 antibody (aPD-1) by IP injection on days 4, 7, 11, and 14 post inoculation.

TABLE 6
In vivo efficacy experimental design.

| Grp. | Drug | Dose | Route | # Doses | Dosing | N |
|---|---|---|---|---|---|---|
| 1 | PBS | — | IT | 5 | q3d | 5 |
| | Isotype Ctrl | 10 mg/kg | IP | cont. | q2d | |
| 2 | exoSTING | 10 ng | IT | 5 | q3d | 5 |
| | Isotype Ctrl | 10 mg/kg | IP | cont. | q2d | |
| 3 | exoSTING | 10 ng | IT | 3 | q3d | 5 |
| | aPD-1 | 10 mg/kg | IP | cont. | | |
| 4 | exoIL-12 | 100 ng | IT | 3 | q2d | 5 |
| | Isotype Ctrl | 10 mg/kg | IP | cont. | | |
| 5 | exoIL-12 | 100 ng | IT | 3 | q2d | 5 |
| | aPD-1 | 10 mg/kg | IP | cont. | | |
| 6 | exoSTING | 10 ng | IT | 3 | q3d | 5 |
| | exoIL-12 | 100 ng | IT | 3 | q2d | |
| | Isotype Ctrl | 10 mg/kg | IP | cont. | | |
| 7 | exoSTING | 10 ng | IT | 3 | q3d | 5 |
| | exoIL-12 | 100 ng | IT | 3 | q2d 2x/ | |
| | aPD-1 | 10 mg/kg | IP | cont. | wk | |

IT = intratumoral injection;
IP = intraperitoneal injection;
q2d = every 2 days.

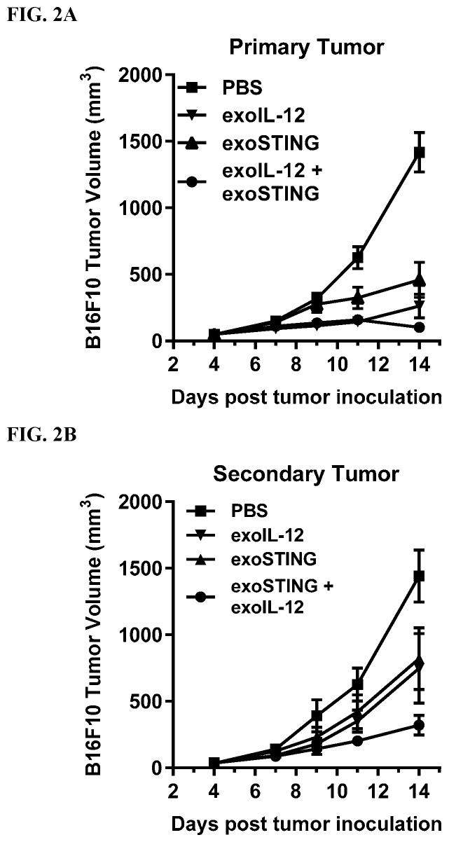

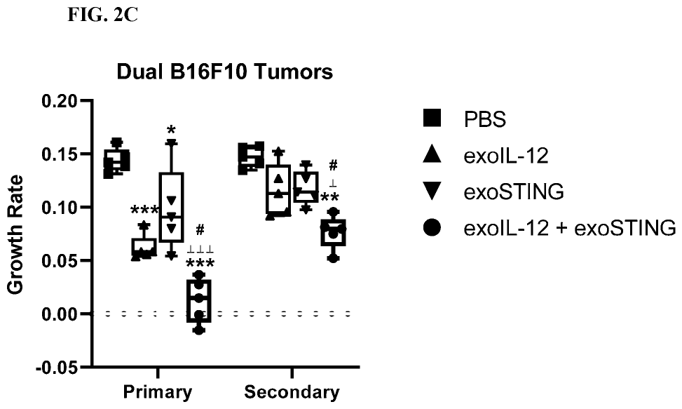

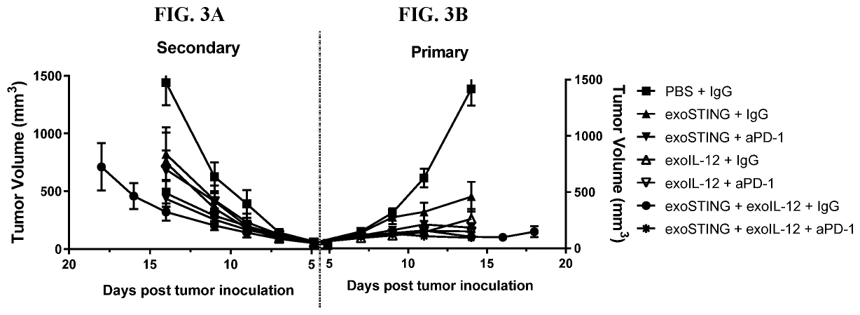

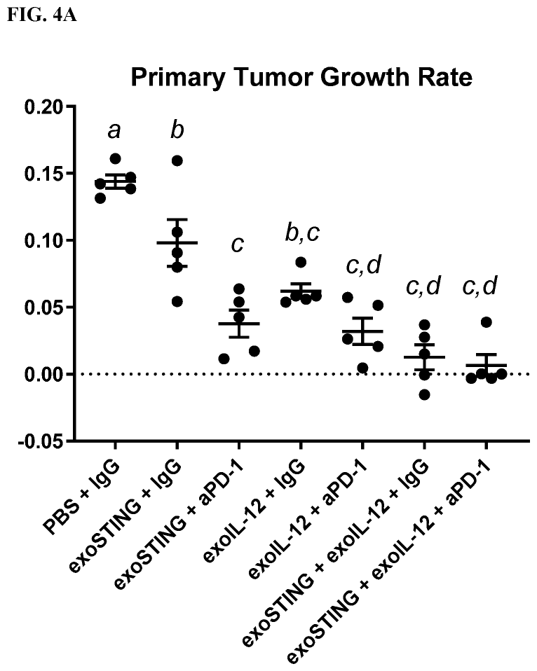

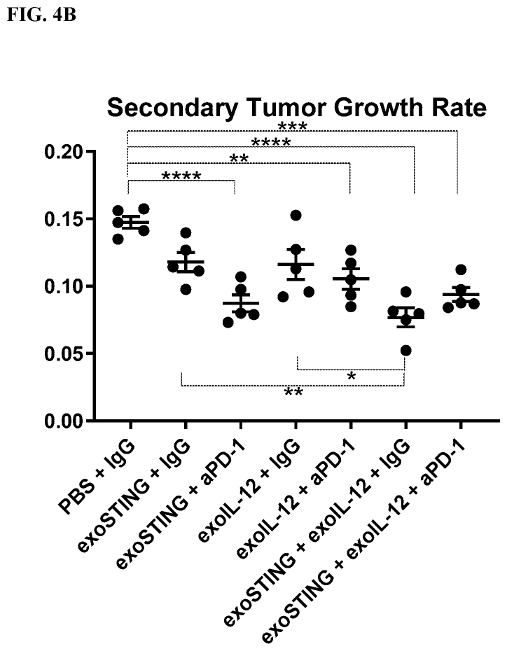

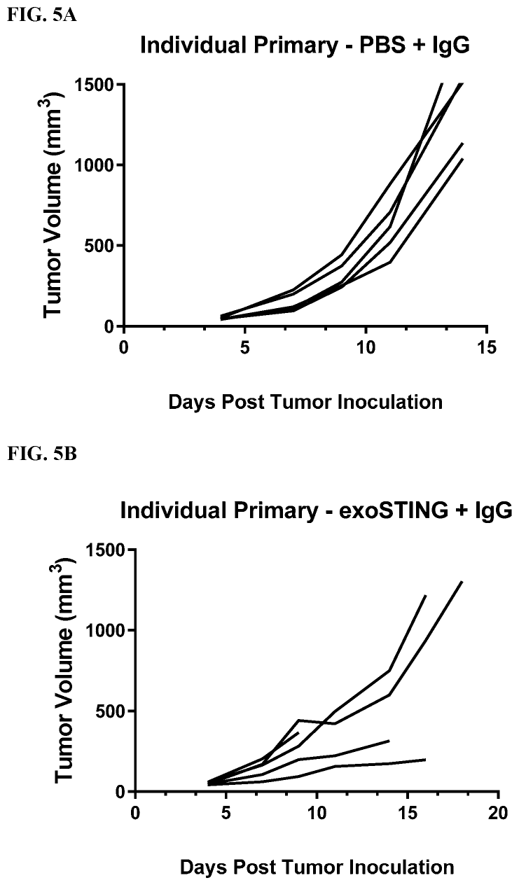

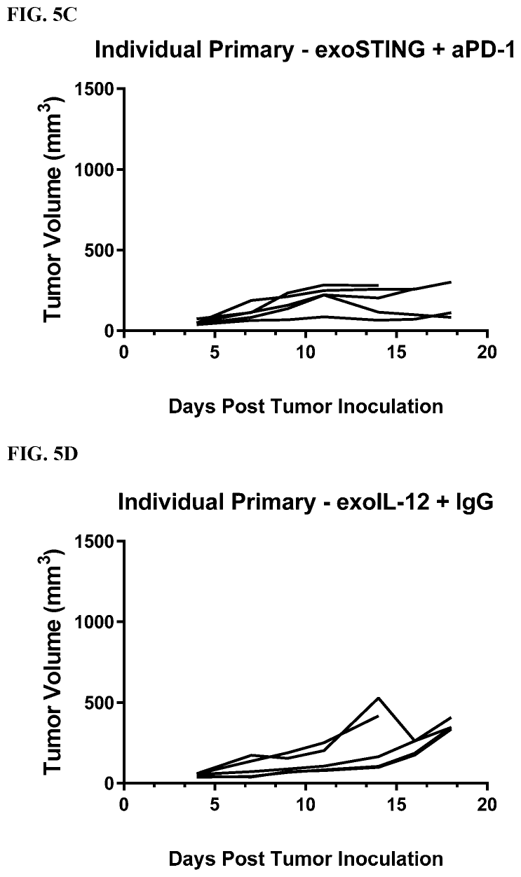

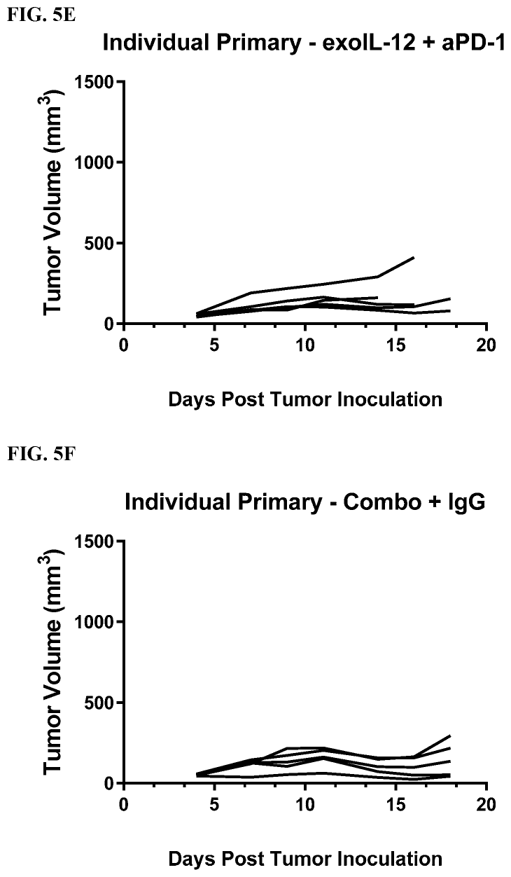

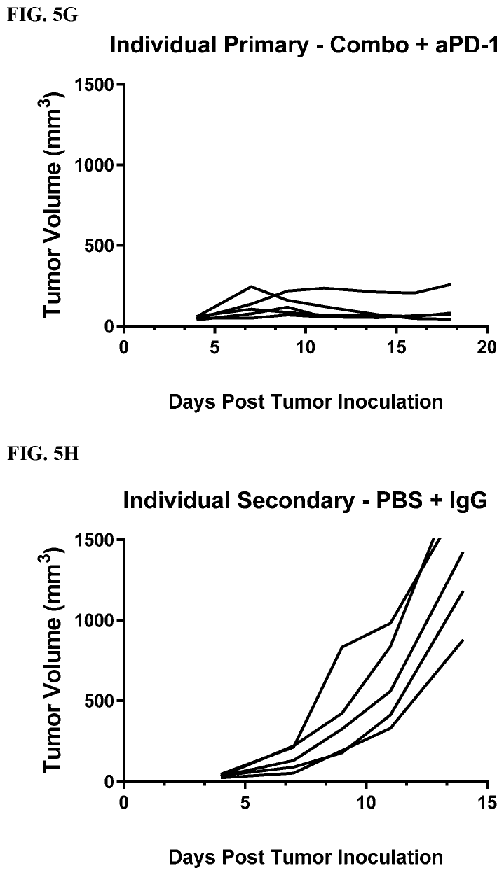

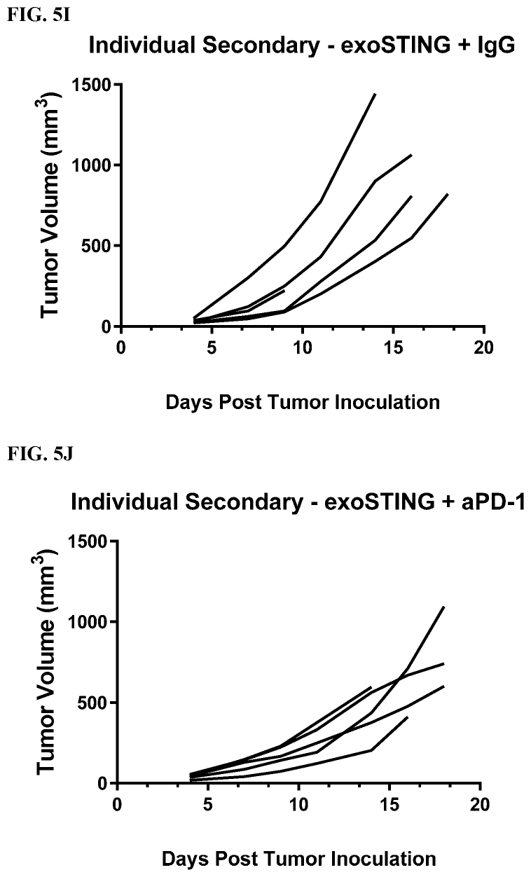

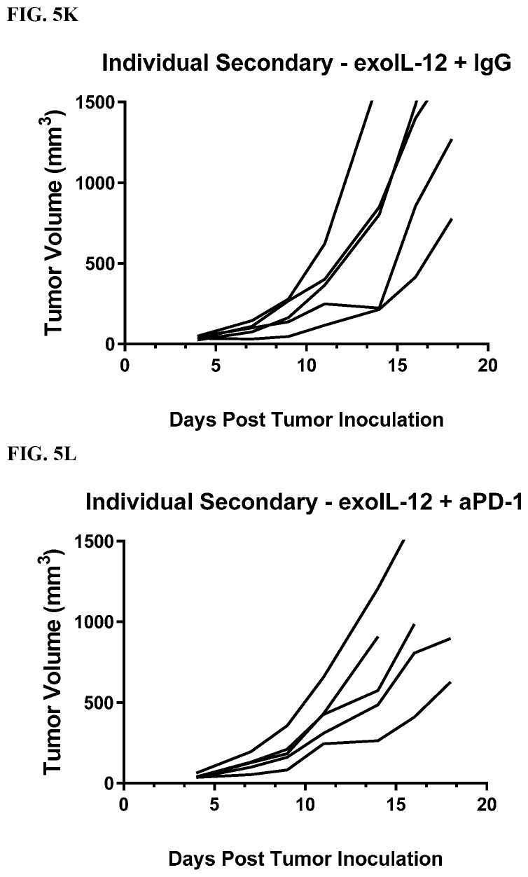

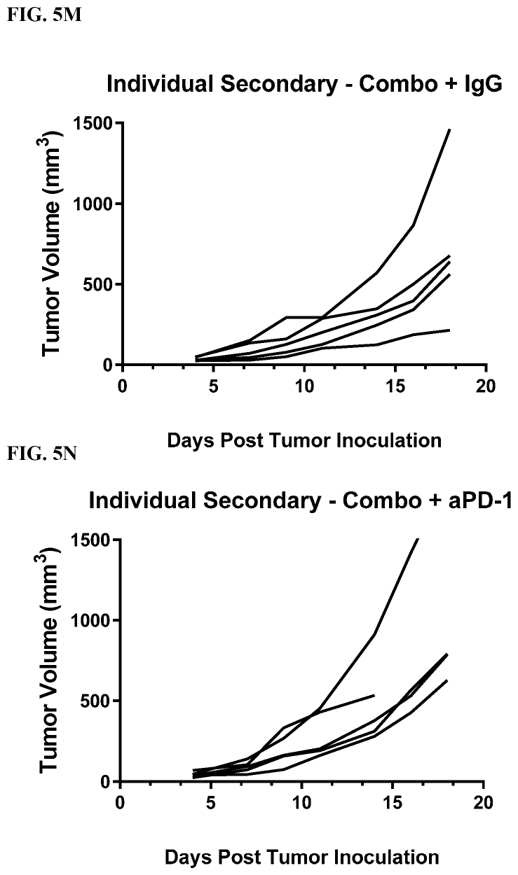

Administration of exosomes with surface-display of IL-12 and/or exosome-loaded STING agonist results in a durable complete response and prevention of tumor growth after re-challenge (FIGS. 2A-2C). Tumor growth was inhibited following administration of exosomes with surface-display of IL-12, exosome-loaded STING agonist, and a combination therapy of exosomes with surface-display of IL-12 and exosome-loaded STING agonist in both the primary tumor (FIG. 2A) and the secondary tumor (FIG. 2B). Tumor growth rate showed a greater reduction following administration of the combination therapy than administration of either exosomes with surface-display of IL-12 or exosome-loaded STING agonist alone (FIG. 2C). Though these effects were more pronounced in the primary tumor, each treatment group resulted in at least a slight decrease in tumor growth rate in the secondary (un-injected) tumor, with a significant decrease in tumor growth rate following combination therapy (FIG. 2C). Tumor size at day 14 was further reduced in both primary and secondary tumors in mice treated with a triple combination of exosomes with surface-display of IL-12, exosome-loaded STING agonist, and an anti-PD-1 antibody (FIGS. 3A-31B). However, the tumor growth rate in both primary and secondary tumors following administration of the triple combination was not statistically different than the growth rates following the combination therapy of exosomes with surface-display of IL-12 and exosome-loaded STING agonist (FIGS. 4A-4B). Individual treatment results are shown in FIGS. 5A-5N.

### Example 3: Administration of Exosomes Comprising Surface-Displayed IL-12 in a Mouse Cancer Model

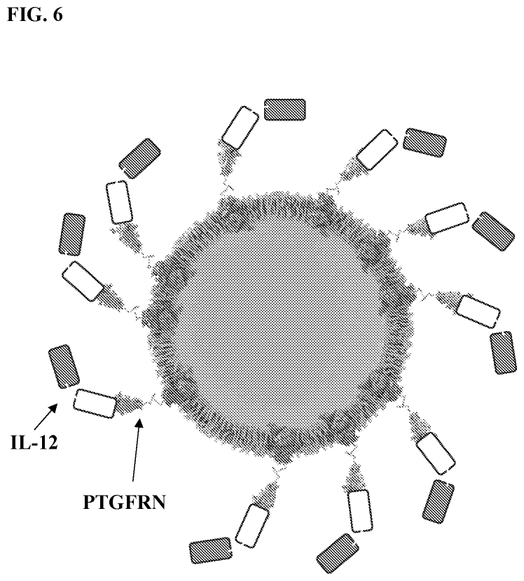

Exosomes with surface-displayed IL-12 were prepared by expressing in exosome-producing cells a fusion construct comprising a single peptide IL-12 (p29 linked by a peptide linker p40) linked to PTGFRN (FIG. 6). Potency was assessed in vitro using human PBMCs or murine splenocytes and in vivo using mouse subcutaneous tumor models. Local versus systemic pharmacology was determined with intratumoral injection in mice and subcutaneous injection in monkeys. All studies were benchmarked against recombinant IL-12 (rIL-12).

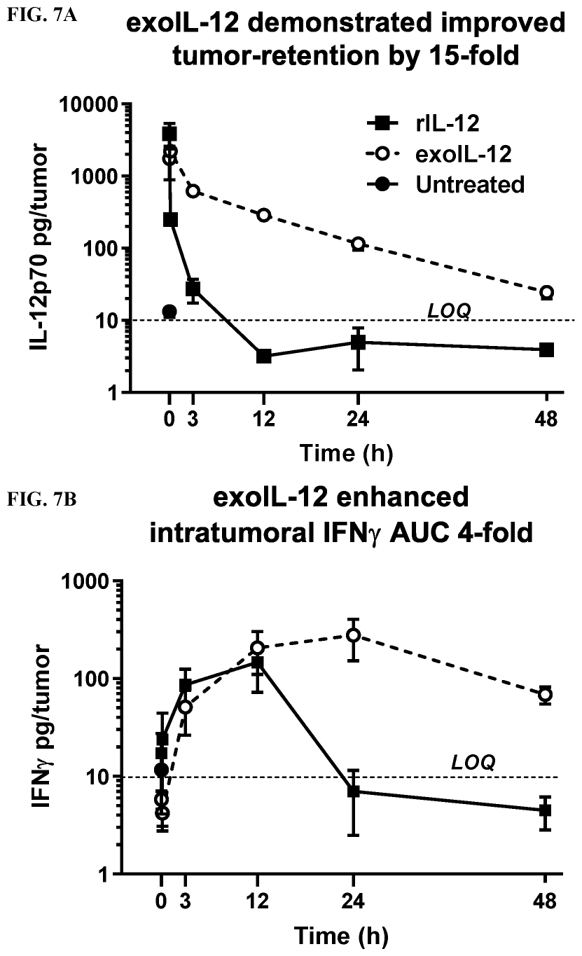

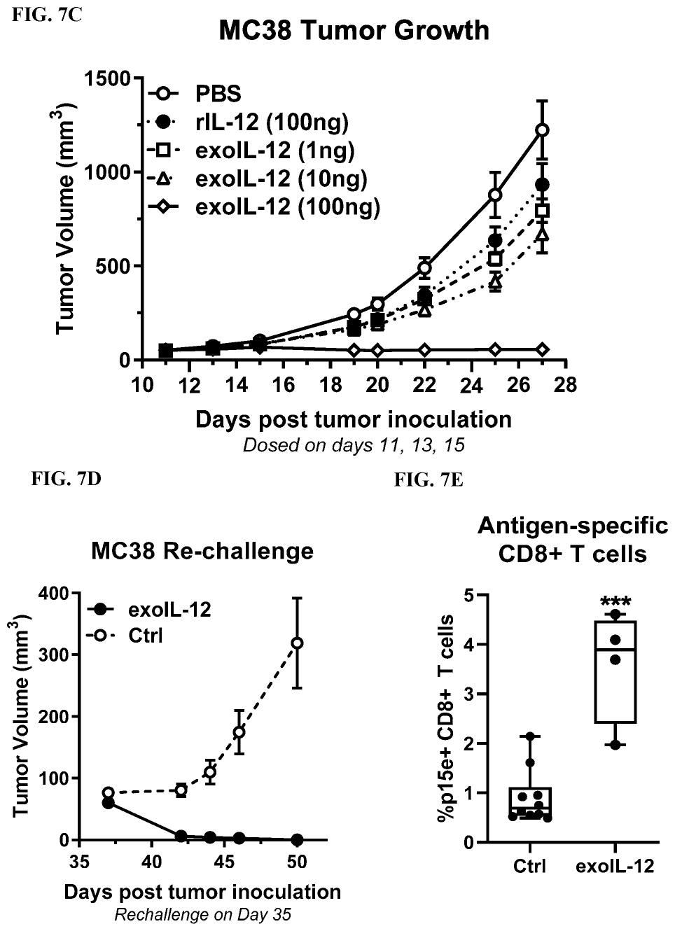

In an MC38 mouse tumor model, intratumoral administration of exosomes with surface-displayed IL-12 (exoIL-12) showed enhanced PK and sustained PD as compared to free recombinant IL-12 and untreated controls. Mice administered exoIL-12 showed increased tumor retention (of about 15-fold) as measured by IL-12p70 concentration per tumor at 3, 12, 24, and 48 hours post administration, compared with free recombinant IL-12 (FIG. 7A). In addition, exoIL-12 administration led to enhanced intratumoral IFN-gamma AUC by about 4-fold as compared to recombinant IL-12 (FIG. 7B). Further, intratumoral administration of exoIL-12 led to a dose dependent reduction in MC38 tumor growth in mice. ExoIL-12 was 100-fold more potent than rIL-12 in tumor growth inhibition, with mice receiving 100 ng exoIL-12 showing little to no tumor growth (FIG. 7C). In the MC38 tumor model, complete responses were observed in 63% of mice treated with exoIL-12; in contrast, rIL-12 resulted in 0% complete responses at an equivalent IL-12 dose. This correlated with dose-dependent increases in tumor antigen-specific CD8+ T cells, which increased nearly 4-fold in exoIL-12 treated mice (FIG. 7E).

Suppression of tumor growth was again observed following MC38 rechallenge (FIG. 7D). Re-challenge studies of exoIL-12 complete responder mice showed no tumor regrowth and depletion of CD8+ T cells completely abrogated antitumor activity of exoIL-12. Following intratumoral administration, exoIL-12 exhibited 10-fold higher intratumoral exposure than rIL-12 and prolonged IFNγ production up to 48 hr. Retained local pharmacology of exoIL-12 was further confirmed using subcutaneous injections in non-human primates.

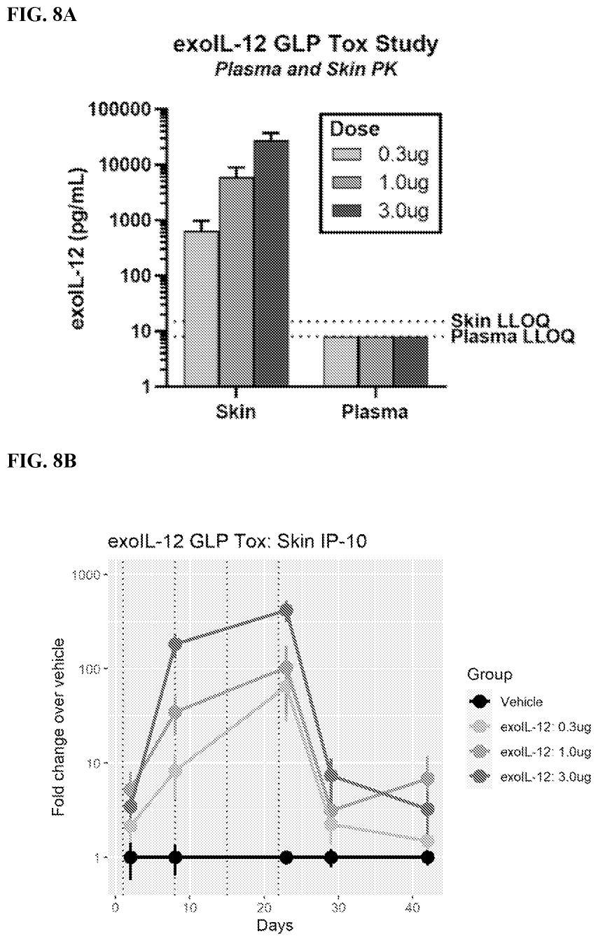

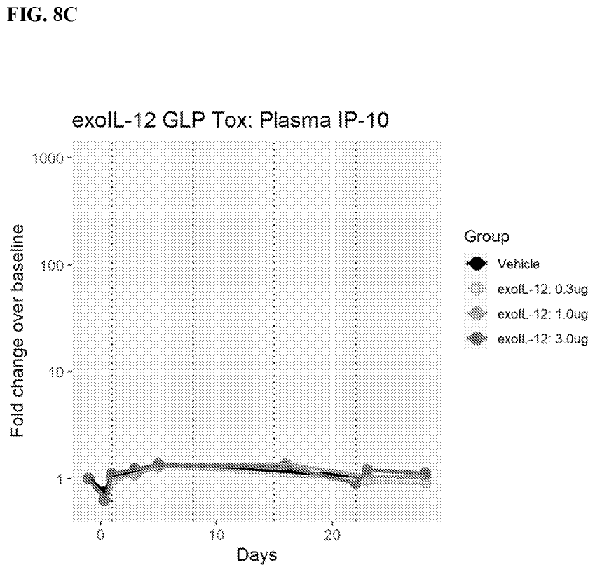

Toxicology analysis revealed that the highest dose tested, 3 μg exoIL-12, was NOAEL (no observable adverse effect level), showing limited plasma levels and dose dependent tissue levels (FIG. 8A). CXCL10/IP-10 expression was observed to be sustained in the skin after a single dose but was undetectable in plasma (FIGS. 8B-8C).

Tumor-restricted pharmacology of exoIL-12 results in superior in vivo efficacy and immune memory without systemic IL-12 exposure and related toxicity. As such, exoIL-12 overcomes key limitations of rIL-12.

### Example 4: Clinical Trial Studying the Safety and Efficacy of Administration of Exosomes Comprising Surface-Displayed IL-12

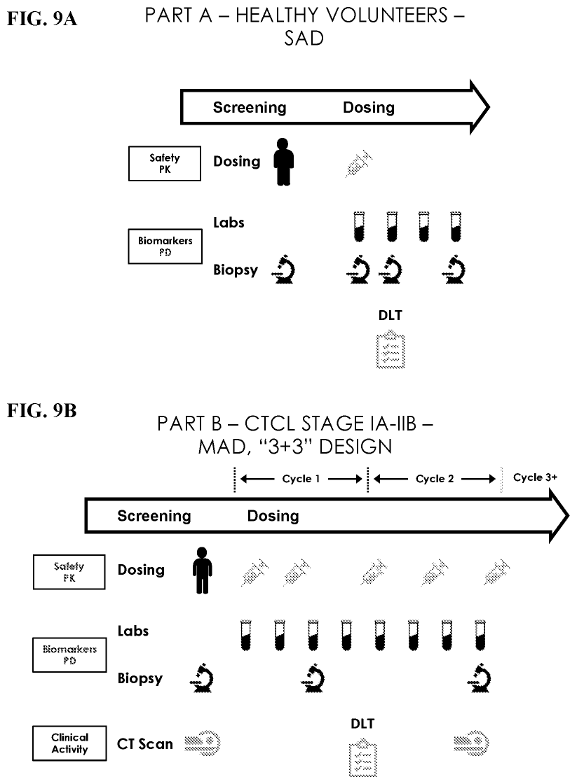

A clinical study will be conducted to test the safety and efficacy of treating a cancer in a human subject by administering engineered exosomes comprising surface displayed IL-12 (FIGS. 9A-9B). In part A, healthy volunteers will be administered varying doses of exoIL-12 and monitored of adverse events and biomarkers (FIG. 9A). In part B, subjects diagnosed with CTCL (stage IA-IIB) will be administered various doses of exoIL-12 and monitored for safety and biomarkers (FIG. 9B). Clinical activity will be monitored by one or more CT scan. Subjects having CTCL, TNBC, melanoma, GBM, MCC, and/or Kaposi sarcoma will be eligible for part B of the trial.

### Example 5: In Vivo Administration of Exosomes with Surface-Display of IL-12 in Combination with Exosome-Loaded STING Agonists

To determine if synergy with exosomes with surface-display of IL-12 and exosome-loaded STING agonist is superior to anti-PD-1 combination therapy, B16F10 mice were subcutaneously inoculated with tumor cells at two different sites (“primary” and “secondary”). At day 6 post inoculation, at which point the mice had developed primary and secondary tumors of about 50 mm³, mice were divided into 7 treatment groups (Table 7). Mice in group 1 were administered empty exosomes by intratumoral (IT) injection (of the primary tumor) on days 6, 8, 9, 10, and 12 post inoculation, and 10 mg/kg of an isotype control by intraperitoneal (IP) injection on days 6, 9, 12, and 16 post inoculation. Mice in group 2 were administered empty exosomes by IT injection (of the primary tumor) on days 6, 8, 9, 10, and 12 post inoculation, and 10 mg/kg of an anti-PD-1 antibody by intraperitoneal (IP) injection on days 6, 9, 12, and 16 post inoculation. Mice in group 3 were administered 100 ng of exosomes loaded with STING agonist by IT injection (of the primary tumor) on days 6, 9, and 12 post inoculation, and 10 mg/kg of an isotype control by IP injection on days 6, 9, 12, and 16 post inoculation. Mice in group 4 were administered 100 ng of exosomes loaded with STING agonist by IT injection (of the primary tumor) on days 6, 9, and 12 post inoculation, and 10 mg/kg of an anti-PD-1 antibody by IP injection on days 6, 9, 12, and 16 post inoculation. Mice in group 5 were administered 100 ng of exosomes loaded with IL-12 by IT injection (of the primary tumor) on days 6, 8, and 10 post inoculation, and 10 mg/kg of an anti-PD-1 antibody by IP injection on days 6, 9, 12, and 16 post inoculation. Mice in group 6 were administered 100 ng of exosomes loaded with STING agonist by IT injection (of the primary tumor) on days 6, 9, and 12 post inoculation; 100 ng of exosomes loaded with IL-12 by IT injection (of the primary tumor) on days 8, 10, and 12 post inoculation. Mice in group 7 were administered 10 ng of exosomes loaded with STING agonist by IT injection (of the primary tumor) on days 6, 9, and 12 post inoculation; 10 ng of exosomes loaded with IL-12 by IT injection (of the primary tumor) on days 8, 10, and 12 post inoculation.

TABLE 7
In vivo efficacy experimental design.

| Grp. | Drug | Dose | Route | # Doses | Dosing | N |
|---|---|---|---|---|---|---|
| 1 | Exosomes | 1E11 | IT | 5 | q3d | 8 |
| | Isotype Ctrl | 10 mg/kg | IP | cont. | q2d | |
| 2 | Exosomes | 1E11 | IT | 5 | q3d | 8 |
| | aPD-1 | 10 mg/kg | IP | cont. | q2d | |
| 3 | exoSTING | 100 ng | IT | 3 | q3d | 8 |
| | Isotype Ctrl | 10 mg/kg | IP | cont. | | |
| 4 | exoSTING | 100 ng | IT | 3 | Q3d | 8 |
| | aPD-1 | 10 mg/kg | IP | cont. | | |
| 5 | exoIL-12 | 100 ng | IT | 3 | q2d | 8 |
| | aPD-1 | 10 mg/kg | IP | cont. | | |
| 6 | exoSTING | 100 ng | IT | 3 | q3d | 8 |
| | exoIL-12 | 100 ng | IT | 3 | q2d | |
| 7 | exoSTING | 10 ng | IT | 3 | q3d | 8 |
| | exoIL-12 | 10 ng | IT | 3 | q2d | |

IT = intratumoral injection;
IP = intraperitoneal injection;
aPD-1 = anti-PD-1 antibody;
q2d = every 2 days;
q3d = every 3 days.

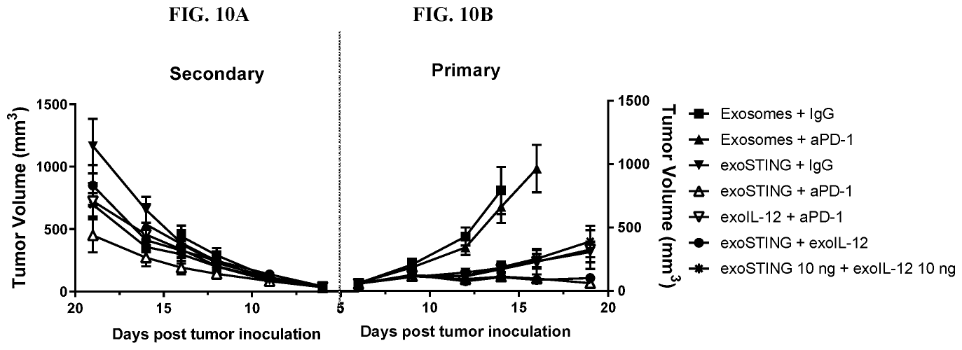

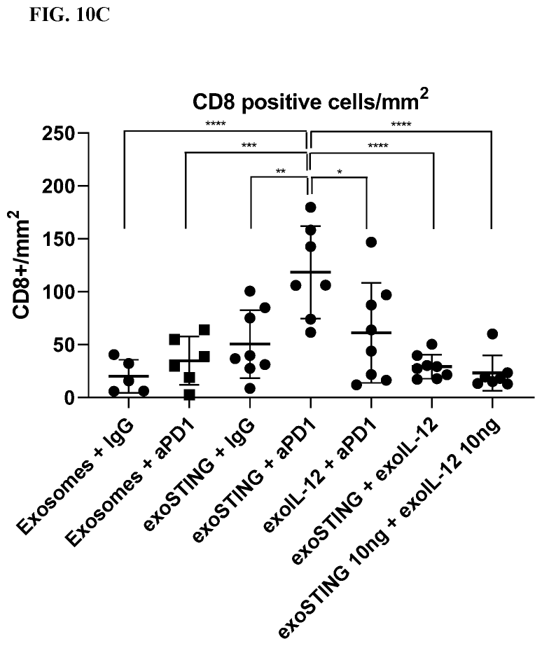

Tumor growth was inhibited following administration of 1) exosome-loaded STING agonist with STING agonist, 2) exosome-loaded with STING agonist in combination with aPD-1, 3) exosomes with surface-display of IL-12 in combination with aPD-1, and 4) a combination therapy of exosomes with surface-display of IL-12 and exosome-loaded STING agonist at 100 ng and 10 ng doses in the primary tumor (FIG. 10B). Though these effects were more pronounced in the primary tumor, each treatment group resulted in at least a slight decrease in tumor growth rate in the secondary (un-injected) tumor, with a significant decrease in tumor growth rate following combination of exosome-loaded with STING agonist and an anti-PD-1 antibody (FIG. 10A). Infiltration of CD8 T-cells into the secondary tumor was significantly higher in exosome-loaded with STING agonist and anti-PD-1 antibody combination, compared to exosomes, exosomes in combination with an anti-PD-1 antibody, exosome-loaded with STING agonist, exosomes with surface-display of IL-12 in combination with an anti-PD-1 antibody or a combination therapy of exosomes with surface-display of IL-12 and exosome-loaded STING agonist at 100 ng and 10 ng doses (FIG. 10C).

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

The present disclosure provides, inter alia, compositions of exosomes encapsulating STING agonists for use as therapeutics. The present disclosure also provides methods of producing exosomes encapsulating STING agonists and methods of administering such exosomes as therapeutics. While various specific aspects have been illustrated and described, the above specification is not restrictive. It will be appreciated that various changes can be made without departing from the spirit and scope of the invention(s). Many variations will become apparent to those skilled in the art upon review of this specification.

What is claimed:

1. A method of treating a tumor in a subject in need thereof comprising administering (i) a composition comprising an extracellular vesicle (EV) and a stimulator of interferon genes protein (STING) agonist in combination with (ii) an interleukin 12 (IL-12) moiety.

2. The method of claim 1, wherein the IL-12 moiety is associated with a second EV.

3. The method of claim 1, wherein the IL-12 moiety is associated with the EV comprising the STING agonist.

4. The method of any one of claims 1 to 3, wherein the tumor is a primary tumor, a secondary tumor, or both a primary tumor and a secondary tumor.

5. The method of any one of claims 1 to 4, wherein the administering reduces the volume of the tumor.

6. The method of any one of claims 1 to 5, wherein the administering reduces the volume of the tumor by at least two fold, at least three fold, at least four fold, at least five fold, at least six fold, at least seven fold, at least nine fold, or at least ten fold compared to the tumor volume after administering either an extracellular vesicle comprising the STING agonist or the IL-12 moiety (“monotherapy”).

7. The method of claim 5 or 6, wherein the administering reduces the volume of the primary tumor.

8. The method of claim 7, wherein the administering is capable of reducing the volume of the primary tumor by at least about 1.5 fold, at least about 2 fold, at least about 3 fold, at least about 4 fold, or at least about 5 fold compared to the monotherapy after day 14 of the administering.

9. The method of any one of claims 4 to 8, wherein the administering reduces the volume of the secondary tumor.

10. The method of claim 9, wherein the administering is capable of reducing the volume of the secondary tumor by at least about 1.5 fold, at least about 1.6 fold, at least about 1.7 fold, at least about 1.8 fold, at least about 1.9 fold, or at least about 2 fold compared to the monotherapy after day 14 of the administering.

11. The method of any one of claims 1 to 10, wherein the administering reduces the growth of the tumor.

12. The method of any one of claims 1 to 11, wherein the administering reduces the growth of the tumor by at least two fold, at least three fold, at least four fold, at least five fold, at least six fold, at least seven fold, at least nine fold, or at least ten fold compared to the tumor volume after administering either an extracellular vesicle comprising the STING agonist or the IL-12 moiety (“monotherapy”).

13. The method of claim 11 or 12, wherein the administering reduces the growth of the primary tumor and/or the secondary tumor.

14. The method of any one of claims 1 to 13, further comprising administering an anti-cancer agent.

15. The method of claim 14, wherein the anti-cancer agent comprises a checkpoint inhibitor.

16. The method of claim 15, wherein the checkpoint inhibitor comprises an anti-PD-1 antibody, an anti-PD-L1 antibody, an anti-CTLA-4 antibody, an anti-LAG-3 antibody, an anti-TIM-3 antibody, or any combination thereof.

17. The method of claim 15, wherein the checkpoint inhibitor is an anti-PD-1 antibody.

18. An extracellular vesicle comprising a STING agonist and an IL-12 moiety.

19. A composition comprising an extracellular vesicle comprising a STING agonist and a second EV comprising an IL-12 moiety.

20. The method of any one of claims 1 to 17, EV of claim 18, or composition of claim 19, wherein the IL-12 moiety is an IL-12 protein, a nucleic acid encoding an IL-12 protein, or a molecule having an IL-12 activity.

21. The method of any one of claims 1 to 17 and 20, EV of claim 18 or 20, or composition of claim 19 or 20, wherein the IL-12 moiety is an IL-12 protein.

22. The method of any one of claims 1 to 17, 20, and 21 or EV of any one of claims 18, 20 and 21, wherein the extracellular vesicle is an exosome, a nanovesicle, an apoptotic body, a microvesicle, a lysosome, an endosome, a liposome, a lipid nanoparticle, a micelle, a multilamellar structure, a revesiculated vesicle, or an extruded cell.

23. The method of any one of claims 1 to 17 and 20 to 22, EV of any one of claims 18 and 20 to 22, or composition of any one of claims 19 to 22, wherein the EV is an exosome.

24. The method of any one of claims 1 to 17 and 20 to 23, EV of any one of claims 18 and 20 to 23, or composition of any one of claims 19 to 23, wherein the STING agonist is associated with the EV.

25. The method of any one of claims 1 to 17 and 20 to 23, EV of any one of claims 18 and 20 to 23, or composition of any one of claims 19 to 23, wherein the STING agonist is encapsulated within the EV.

26. The method of any one of claims 1 to 17 and 20 to 23, EV of any one of claims 18 and 20 to 23, or composition of any one of claims 19 to 23, wherein the STING agonist is linked to a lipid bilayer of the EV, optionally by a linker.

27. The method of any one of claims 1 to 17 and 20 to 26, EV of any one of claims 18 and 20 to 26, or composition of any one of claims 19 to 26, wherein the EV overexpresses a Prostaglandin F2 receptor negative regulator (PTGFRN) protein.

28. The method, EV or composition of claim 27, wherein the STING agonist is not linked to the PTGFRN protein.

29. The method of any one of claims 1 to 17 and 20 to 28, EV of any one of claims 18 and 20 to 28, or composition of any one of claims 19 to 28, wherein the extracellular vesicle is produced by a cell that overexpresses a PTGFRN protein.

30. The method of any one of claims 1 to 17 and 20 to 28, EV of any one of claims 18 and 20 to 28, or composition of any one of claims 19 to 28, wherein the extracellular vesicle further comprises a ligand, a cytokine, or an antibody.

31. The method, EV or composition of claim 30, wherein the antibody comprises an antagonistic antibody and/or an agonistic antibody.

32. The method of any one of claims 1 to 17 and 20 to 31, EV of any one of claims 18 and 20 to 31, or composition of any one of claims 19 to 31, wherein the STING agonist is a cyclic dinucleotide.

33. The method of any one of claims 1 to 17 and 20 to 31, EV of any one of claims 18 and 20 to 31, or composition of any one of claims 19 to 31, wherein the STING agonist is a non-cyclic dinucleotide.

34. The method of any one of claims 1 to 17 and 20 to 33, EV of any one of claims 18 and 20 to 33, or composition of any one of claims 19 to 33, wherein the STING agonist comprises a lipid-binding tag.

35. The method of any one of claims 1 to 17 and 20 to 35, EV of any one of claims 18 and 20 to 35, or composition of any one of claims 19 to 35, wherein the STING agonist is physically and/or chemically modified.

36. The method, EV or composition of claim 35, wherein the modified STING agonist has a polarity and/or a charge different from the corresponding unmodified STING agonist.

37. The method of any one of claims 1 to 17 and 20 to 36, EV of any one of claims 18 and 20 to 36, or composition of any one of claims 19 to 36, wherein the concentration of the STING agonist is about 0.01 μM to 100 μM.

38. The method of any one of claims 1 to 17 and 20 to 37, EV of any one of claims 18 and 20 to 37, or composition of any one of claims 19 to 37, wherein the concentration of the STING agonist is about 0.01 μM to 0.1 μM, 0.1 μM to 1 μM, 1 μM to 10 μM, 10 μM to 50 μM, or 50 μM to 100 μM.

39. The method, EV, or composition of claim 38, wherein the concentration of the STING agonist in the EV is about 1 μM to 10 μM.

40. The method of any one of claims 1 to 17 and 20 to 39, EV of any one of claims 18 and 20 to 39, or composition of any one of claims 19 to 39, wherein the STING agonist comprises: 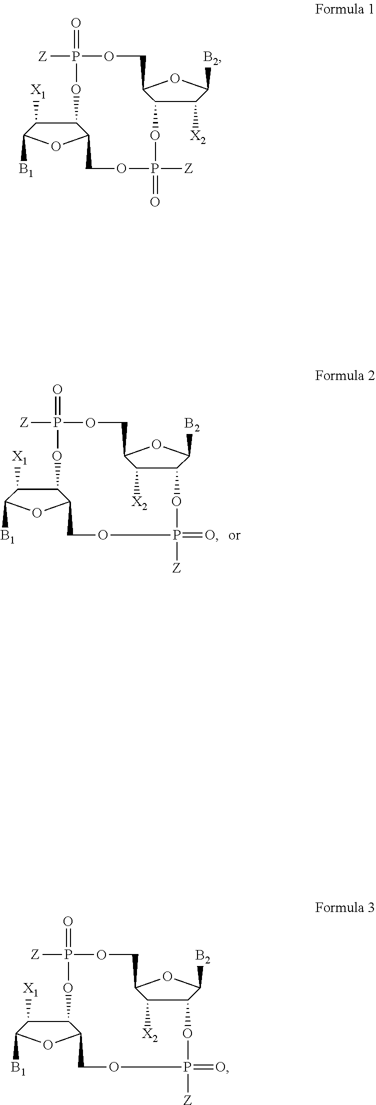
wherein:
X₁ is H, OH, or F;
X₂ is H, OH, or F;
Z is OH, OR₁, SH or SR₁, wherein:
i) R₁ is Na or NH₄, or
ii) R₁ is an enzyme-labile group which provides OH or SH in vivo such as pivaloyloxymethyl;
Bi and B2 are bases chosen from: 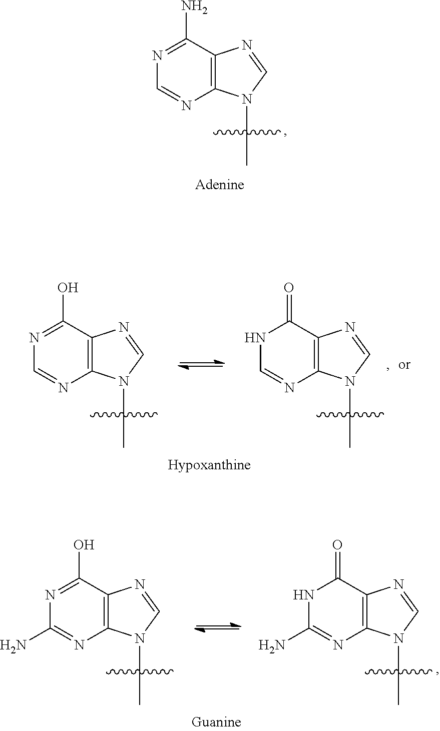 [SMILES: *n1cnc2c(N)ncnc21 ; *n1cnc2c(O)ncnc21 ; *n1cnc2c(=O)[nH]cnc21 ; *n1cnc2c(O)nc(N)nc21 ; *n1cnc2c(=O)[nH]c(N)nc21]
With the proviso that:
in Formula (I): X₁ and X₂ are not OH,
in Formula (II): when X₁ and X₂ are OH, B₁ is not Adenine and B₂ is not Guanine, and
in Formula (III): when X₁ and X₂ are OH, B₁ is not Adenine, B₂ is not Guanine and Z is not OH, or a pharmaceutically acceptable salt thereof.

41. The method of any one of claims 1 to 17 and 20 to 40, EV of any one of claims 18 and 20 to 40, or composition of any one of claims 19 to 40, wherein the STING agonist is selected from the group consisting of: 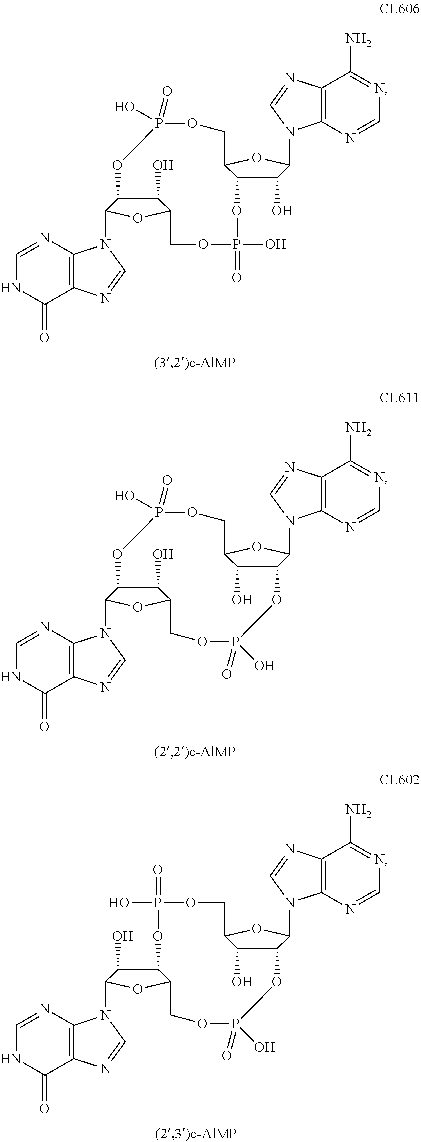 [SMILES: Nc1ncnc2c1ncn2C1OC2COP(=O)(O)O[C@H]3C(n4cnc5c(=O)[nH]cnc54)OC(COP(=O)(O)O[C@H]2[C@H]1O)[C@H]3O ; Nc1ncnc2c1ncn2C1OC2COP(=O)(O)O[C@H]3C(n4cnc5c(=O)[nH]cnc54)OC(COP(=O)(O)O[C@@H]1[C@@H]2O)[C@H]3O ; Nc1ncnc2c1ncn2C1OC2COP(=O)(O)O[C@@H]3C(COP(=O)(O)O[C@@H]1[C@@H]2O)OC(n1cnc2c(=O)[nH]cnc21)[C@@H]3O] 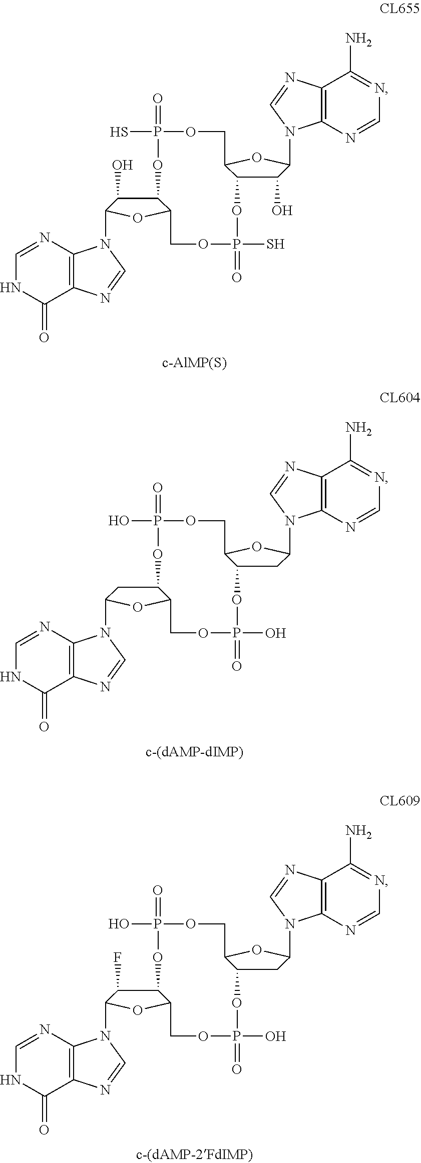 [SMILES: Nc1ncnc2c1ncn2C1OC2COP(=O)(S)O[C@@H]3C(COP(=O)(S)O[C@H]2[C@H]1O)OC(n1cnc2c(=O)[nH]cnc21)[C@@H]3O ; Nc1ncnc2c1ncn2C1C[C@@H]2OP(=O)(O)OCC3OC(n4cnc5c(=O)[nH]cnc54)C[C@@H]3OP(=O)(O)OCC2O1 ; Nc1ncnc2c1ncn2C1C[C@@H]2OP(=O)(O)OCC3OC(n4cnc5c(=O)[nH]cnc54)[C@H](F)[C@@H]3OP(=O)(O)OCC2O1] 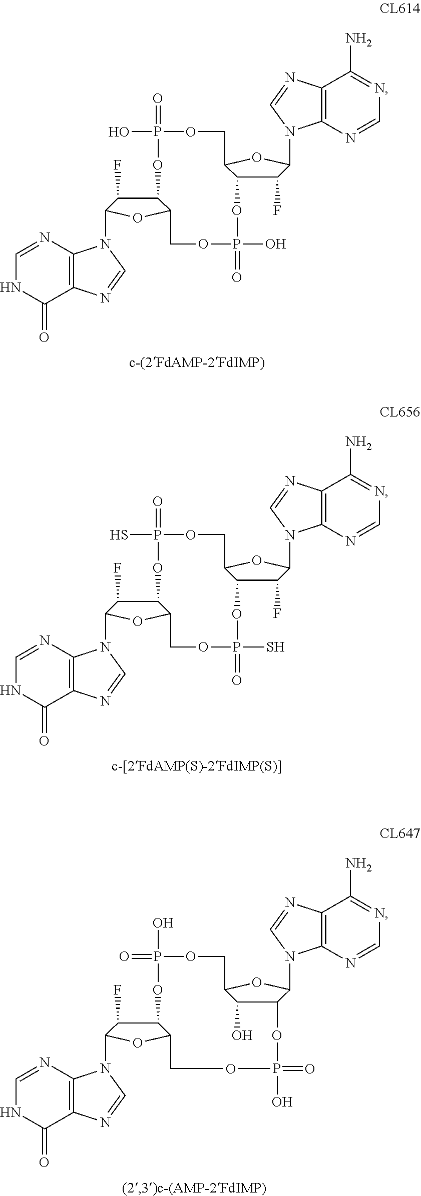 [SMILES: Nc1ncnc2c1ncn2C1OC2COP(=O)(O)O[C@@H]3C(COP(=O)(O)O[C@H]2[C@H]1F)OC(n1cnc2c(=O)[nH]cnc21)[C@@H]3F ; Nc1ncnc2c1ncn2C1OC2COP(=O)(S)O[C@@H]3C(COP(=O)(S)O[C@H]2[C@H]1F)OC(n1cnc2c(=O)[nH]cnc21)[C@@H]3F ; Nc1ncnc2c1ncn2C1OC2COP(=O)(O)O[C@@H]3C(COP(=O)(O)OC1[C@@H]2O)OC(n1cnc2c(=O)[nH]cnc21)[C@@H]3F] 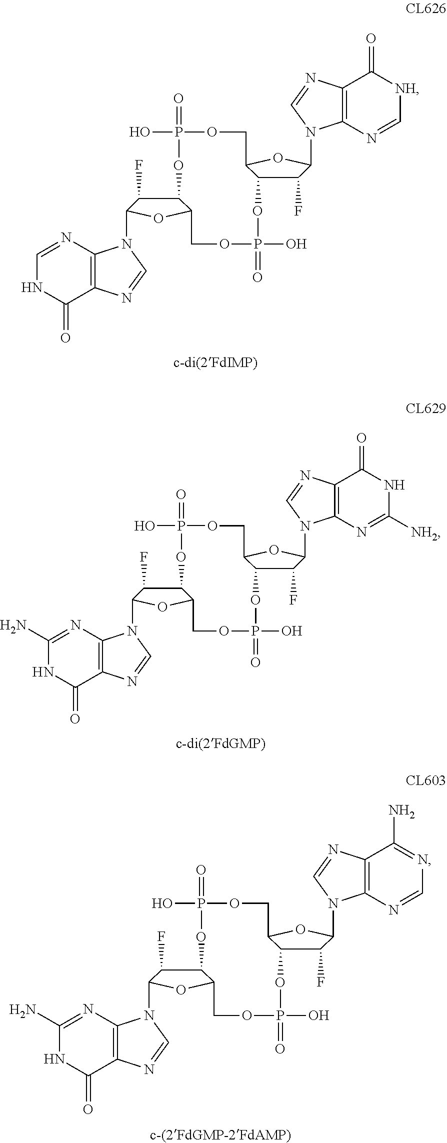 [SMILES: O=c1[nH]cnc2c1ncn2C1OC2COP(=O)(O)O[C@@H]3C(COP(=O)(O)O[C@H]2[C@H]1F)OC(n1cnc2c(=O)[nH]cnc21)[C@@H]3F ; Nc1nc2c(ncn2C2OC3COP(=O)(O)O[C@@H]4C(COP(=O)(O)O[C@H]3[C@H]2F)OC(n2cnc3c(=O)[nH]c(N)nc32)[C@@H]4F)c(=O)[nH]1 ; Nc1nc2c(ncn2C2OC3COP(=O)(O)O[C@@H]4C(COP(=O)(O)O[C@H]3[C@H]2F)OC(n2cnc3c(N)ncnc32)[C@@H]4F)c(=O)[nH]1] 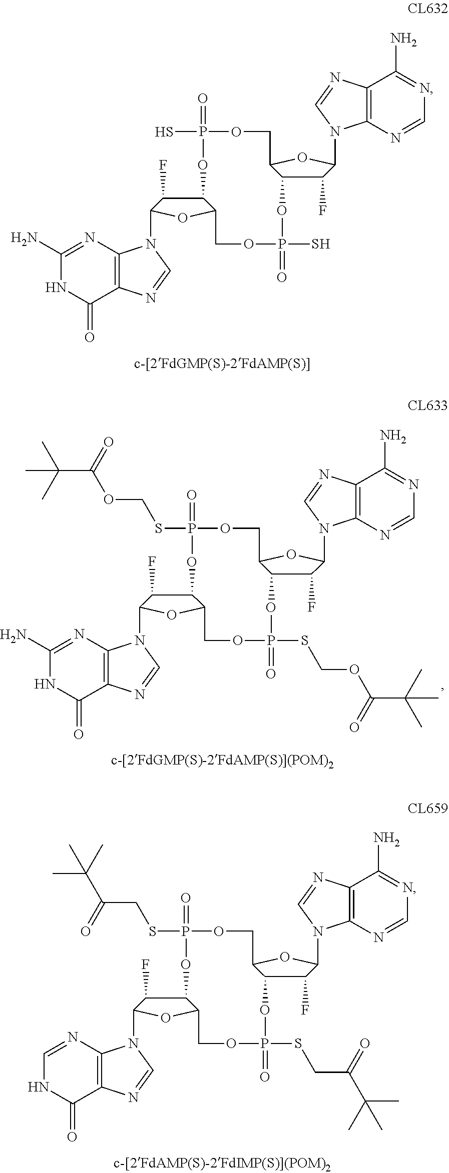 [SMILES: Nc1nc2c(ncn2C2OC3COP(=O)(S)O[C@@H]4C(COP(=O)(S)O[C@H]3[C@H]2F)OC(n2cnc3c(N)ncnc32)[C@@H]4F)c(=O)[nH]1 ; CC(C)(C)C(=O)OCSP1(=O)OCC2OC(n3cnc4c(=O)[nH]c(N)nc43)[C@H](F)[C@@H]2OP(=O)(SCOC(=O)C(C)(C)C)OCC2OC(n3cnc4c(N)ncnc43)[C@H](F)[C@@H]2O1 ; CC(C)(C)C(=O)CSP1(=O)OCC2OC(n3cnc4c(=O)[nH]cnc43)[C@H](F)[C@@H]2OP(=O)(SCC(=O)C(C)(C)C)OCC2OC(n3cnc4c(N)ncnc43)[C@H](F)[C@@H]2O1]
and a pharmaceutically acceptable salt thereof.

42. The EV of any one of claims 18 and 20 to 41, wherein the IL-12 moiety is linked to a scaffold moiety.

43. The method of any one of claims 2 to 17 and 20 to 41 or composition of any one of claims 19 to 41, wherein the second EV comprises a scaffold moiety.

44. The method or composition of claim 43, wherein the IL-12 moiety is linked to the scaffold moiety.

45. The EV of claim 42 or method or composition of claim 43, wherein the scaffold moiety comprises a PTGFRN protein.

46. The EV, method, or composition of claim 45, wherein the PTGFRN protein comprises SEQ ID NO: 33.

47. The EV, method, or composition of claim 46, wherein the PTGFRN protein comprises at least about 70%, at least about 85%, at least about 90%, at least about 95%, at least about 96%, at least about 97%, at least about 98%, at least about 99%, or about 100% identical to SEQ ID NO: 1.

48. The EV, method, or composition of claim 47, wherein the PTGFRN protein comprises the amino acid sequence as set forth in SEQ ID NO: 1.

49. A pharmaceutical composition comprising the EV of any one of claims 18 to 42 and 45 to 48 or composition of any one of claims 19 to 41 and 43 to 48 and a pharmaceutically acceptable carrier.

50. A kit comprising the composition of claim 49 and instructions for use.

51. The method of any one of claims 1 to 17, 20 to 41, 43 to 48, wherein the administration is parenterally, orally, intravenously, intramuscularly, intra-tumorally, intraperitoneally, or via any other appropriate administration route.

52. The method of claim 51, wherein the administration is intratumoral.
